(19) United States
(12) Patent Application Publication
(10) Pub. No.: US 2025/0011799 A1
(43) Pub. Date: Jan. 9, 2025

(54) PROBIOTIC PLASMIDS AND USE OF SAME

(71) Applicants: THE WESTMEAD INSTITUTE FOR MEDICAL RESEARCH, Westmead, New South Wales (AU); WESTERN SYDNEY LOCAL HEALTH DISTRICT, Westmead, New South Wales (AU)

(72) Inventors: Muhammad KAMRUZZAMAN, Westmead, New South Wales (AU); Jonathan IREDELL, Westmead, New South Wales (AU)

(21) Appl. No.: 18/713,389

(22) PCT Filed: Nov. 25, 2022
(86) PCT No.: PCT/AU2022/051414

§ 371 Date: May 24, 2024

(30) Foreign Application Priority Data
Nov. 25, 2021 (AU) 2021903809

(51) Int. Cl. C12N 15/70; A01N 63/20; A01N 63/22; A01P 1/00; A23C 9/123; A61K 35/00; A61K 35/741

(52) U.S. Cl. CPC C12N 15/70; A01N 63/20; A01N 63/22; A01P 1/00; A23C 9/123; A61K 35/741; A61K 2035/115

(57) ABSTRACT

The present disclosure relates generally to conjugative plasmids comprising multiple entry exclusion systems derived from plasmids harbouring genes which confer undesirable traits of interest, such as pathogenic traits like antimicrobial resistance (AMR), virulence determinants, metal resistance or other undesirable traits, and the use of those probiotic plasmids to protect bacterial populations (e.g., present in the gut, in the environment etc) from acquiring plasmids which confer the undesirable traits and thereby preventing spread of the undesirable traits (e.g., AMR, virulence or metal resistance etc) within the microbiota.

### CROSS-REFERENCE TO RELATED APPLICATIONS

The present application claims priority from Australian Provisional Patent Application No 2021903809 filed on 25 Nov. 2021, the content of which is incorporated herein by reference in its entirety.

### TECHNICAL FIELD

The present disclosure relates generally to conjugative plasmids comprising multiple entry exclusion systems derived from plasmids harbouring genes which confer undesirable traits of interest, such as pathogenic traits like antimicrobial resistance (AMR), virulence determinants, metal resistance or other undesirable traits, and the use of those probiotic plasmids to protect bacterial populations (e.g., present in the gut, in the environment etc) from acquiring plasmids which confer the undesirable traits and thereby preventing spread of the undesirable traits (e.g., AMR, virulence or metal resistance etc) within the microbiota.

### BACKGROUND

Antimicrobials underpin many facets of modern medicine, including treatment of infections, prevention of infection during surgical procedures and protection of immunocompromised patients from infection. However, over the past several decades, the use of antibiotics and other antimicrobials in human medicine, animal husbandry, veterinary practice and aquaculture has increased dramatically. Despite the growing trend towards improved antimicrobial stewardship in many countries, the historical overuse of antimicrobial agents has selected for many mutant strains of bacteria which are resistant to the commonly used antimicrobials. As a result, one of the major threats facing society is the rise in number of antimicrobial-resistant (AMR) bacteria, such as Escherichia coli, Klebsiella pneumoniae and Salmonella. The ability of these bacteria to resist the effect of antibiotics is largely mediated by invasive plasmids carrying antimicrobial resistance genes (ARG), which, once acquired, cannot be removed due to the use of ‘addiction systems’. Similarly, virulence factors are bacteria-associated molecules that are required for a bacterium to cause disease while infecting eukaryotic hosts such as humans. A large number of virulence factors encoded by bacterial pathogens are also plasmid borne. For example, plasmids in Salmonella, Shigella, E. coli and Yersinia species carry virulence genes (Pilla and Tang 2018). In short, virulence factors important for microbial pathogenesis and virulence plasmids which encode these factors play an important role in disease. Virulence plasmids help bacteria infect humans, animals, and plants by a variety of mechanisms. Some virulence factors are toxins that damage or kill animal cells, others help bacteria to attach to and invade animal cells, whereas yet others protect bacteria from the immune system.

Many pathogenic bacteria carry genes for AMR, virulence or metal resistance on extrachromosomal mobile genetic elements such as plasmids. Large plasmids efficiently transfer between bacteria by cell-cell contact (conjugation) and, once acquired, ensure their own survival in bacterial populations through addiction systems. Due to their self-transmissible property, bacteria can acquire those plasmids quickly from exposure to the environment where AMR or virulence or metal resistance is endemic or prevalent, e.g. hospital, clinics, contamination sites, environments with metal toxicity etc. Once acquired, these plasmids are unlikely to be lost or removed due to their addictive properties. It is therefore highly desirable to protect bacteria from the AMR or virulence plasmid acquisition in the first place in order to reduce pathogenic plasmid burden and control spread.

At present, the strategies for combating virulence/AMR spread are limited. Plasmid curing (in which AMR plasmids are removed from bacterial populations), and anti-plasmid approaches are being explored for lowering ARG prevalence and rendering bacteria susceptible to antibiotics. However, at present, no plasmid curing treatment options are approved and available for use. In fact, there are very few curing mechanisms that have been tested in vivo, even in experimental models. Although research efforts towards plasmid curing approaches have come a long way, each of the strategies discussed above has their own set of limitations and require further research before a safe and effective treatment approach can be made available. Thus, there remains a need for addressing AMR burden and spread.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

The present disclosure is based, inter alia, on a recognition by the inventors that there is a need for novel strategies to combat the prevalence of certain traits in bacterial populations, such as AMR, bacterial virulence and metal resistance as examples, particularly in a clinical or bioremediation context. To this end, the inventors have developed a novel strategy to prevent the acquisition and/or spread of said plasmids by bacterial populations using recombinant conjugative plasmids comprising multiple entry exclusion systems (EES). Plasmid replication incompatibility (Inc) and EES are two plasmid-borne properties that can inhibit the transfer of plasmids to the recipient bacteria carrying identical or closely-related plasmids. The EES specifically is present in most known conjugative plasmid types and can inhibit the entry of closely-related plasmids into a recipient bacteria by 10 to 10,0000 fold, depending on the plasmid type. The present inventors have exploited these plasmid-borne properties to develop probiotic plasmids that can protect the microbiome population from acquiring AMR or virulence plasmids. Specifically, the present inventors have developed probiotic plasmids comprising a plurality of variant EES from AMR plasmids of interest. Plasmids of incompatibility group F are the largest group of the AMR plasmids and typically house multiple replicons. Using their novel approach, the inventors constructed protective probiotic plasmids carrying three genetic variants of the IncF EES gene. The inventors tested the novel probiotic plasmids in in vitro conjugation experiments and found that bacteria carrying these probiotic plasmids can protect >99.99% of their population from the acquisition of different IncF plasmid variants harbouring AMR. The inventors also demonstrated that increased expression of the EES gene (e.g., using an inducible promoter) can significantly increase the protection against invasive AMR plasmids.

In one example, the present disclosure provides a recombinant plasmid comprising entry exclusion systems (EES) from a plurality of plasmid variants, where the plasmid variants comprise a gene conferring a trait of interest. In one example, the recombinant plasmid is a conjugative plasmid.

In one example, the trait of interest is a trait that is undesirable in a bacterial population (e.g., a pathogenic trait). For example, the trait of interest may be antimicrobial resistance (AMR), bacterial virulence, or heavy metal resistance. In one example, the trait is AMR. In one example, the trait is bacterial virulence.

In one example, the recombinant plasmid (e.g., recombinant conjugative plasmid) comprises EES from two plasmid variants comprising a gene conferring the trait of interest (e.g., from a pathogenic plasmid).

In one example, the recombinant plasmid (e.g., recombinant conjugative plasmid) comprises EES from at least three plasmid variants comprising a gene conferring the trait of interest (e.g., from a pathogenic plasmid).

The EES in the recombinant plasmid (e.g., recombinant conjugative plasmid) may be from different incompatibility groups. Alternatively, the EES in the recombinant conjugative plasmid may be from the same incompatibility group.

In one example, one or more of the EES are from a plasmid variant from an incompatibility group (Inc) selected from IncF, IncI, IncA, IncC, IncL, IncM, IncN, IncX and IncH, or plasmids with any other known and unknown Inc groups. For example, one or more of the EES are from plasmid variants of incompatibility group F (IncF). In some examples, a plurality of EES in the recombinant conjugative plasmid are from plasmid variants of IncF (e.g., traS gene variants). In another example, all of the EES in the recombinant conjugative plasmid are from plasmid variants of IncF (e.g., all of the EES are traS gene variants).

In one example, the recombinant plasmid (e.g., recombinant conjugative plasmid) comprises one or more of:
an EES comprising a polynucleotide sequence which is at least 70% identical (e.g., at least 75% identical, or at least 80% identical, or at least 85% identical) to the sequence set forth in SEQ ID NO: 1 (traS_F variant);
an EES comprising a polynucleotide sequence which is at least 70% identical (e.g., at least 75% identical, or at least 80% identical, or at least 85% identical) to the sequence set forth in SEQ ID NO: 2 (traS_R100 variant); and/or
an EES comprising a polynucleotide sequence which is at least 70% identical (e.g., at least 75% identical, or at least 80% identical, or at least 85% identical) to the sequence set forth in SEQ ID NO: 3 (traS_SLT variant).

In another example, the recombinant conjugative plasmid comprises one or more of:
an EES comprising a polynucleotide sequence which is at least 90% identical to the sequence set forth in SEQ ID NO: 1 (traS_F variant);
an EES comprising a polynucleotide sequence which is at least 90% identical to the sequence set forth in SEQ ID NO: 2 (traS_R100 variant); and/or
an EES comprising a polynucleotide sequence which is at least 90% identical to the sequence set forth in SEQ ID NO: 3 (traS_SLT variant).

In one example, the recombinant conjugative plasmid comprises one or more of:
an EES comprising a polynucleotide sequence which is at least 95% identical (e.g., at least 96% identical, or at least 97% identical, or at least 98% identical, or at least 99% identical) to the sequence set forth in SEQ ID NO: 1 (traS_F variant);
an EES comprising a polynucleotide sequence which is at least 95% identical (e.g., at least 96% identical, or at least 97% identical, or at least 98% identical, or at least 99% identical) to the sequence set forth in SEQ ID NO: 2 (traS_R100 variant); and/or
an EES comprising a polynucleotide sequence which is at least 95% identical (e.g., at least 96% identical, or at least 97% identical, or at least 98% identical, or at least 99% identical) to the sequence set forth in SEQ ID NO: 3 (traS_SLT variant).

In yet another example, the recombinant plasmid (e.g., recombinant conjugative plasmid) comprises one or more of:
an EES comprising a polynucleotide sequence set forth in SEQ ID NO: 1 (traS_F variant);
an EES comprising a polynucleotide sequence set forth in SEQ ID NO: 2 (traS_R100 variant); and/or
an EES comprising a polynucleotide sequence set forth in SEQ ID NO: 3 (traS_SLT variant).

In some examples, the recombinant plasmid (e.g., recombinant conjugative plasmid) comprises an EES variant designated herein as the traS_F variant. In some examples, the recombinant plasmid (e.g., recombinant conjugative plasmid) comprises an EES variant designated herein as the traS_R100 variant. In some examples, the recombinant plasmid (e.g., recombinant conjugative plasmid) an EES variant designated herein as the traS_SLT variant. For example, the recombinant plasmid (e.g., recombinant conjugative plasmid) may comprise an EES variant designated herein as the traS_F variant and an EES variant designated herein as the traS_R100 variant. For example, the recombinant plasmid (e.g., recombinant conjugative plasmid) may comprise an EES variant designated herein as the traS_F variant and an EES variant designated herein as the traS_SLT variant. For example, the recombinant plasmid (e.g., recombinant conjugative plasmid) may comprise an EES variant designated herein as the traS_100 variant and an EES variant designated herein as the traS_SLT variant. For example, the recombinant plasmid (e.g., recombinant conjugative plasmid) may comprise an EES variant designated herein as the traS_F variant, an EES variant designated herein as the traS_100 variant, and an EES variant designated herein as the traS_SLT variant.

Alternatively, or in addition, the recombinant plasmid (e.g., recombinant conjugative plasmid) comprises one or more EES from a plasmid variant of incompatibility group L (IncL), incompatibility group C (IncC), incompatibility group M (IncM) and/or incompatibility group A (IncA). In one example, the plasmid comprises an EES from a plasmid variant of incompatibility group L (IncL). In one example, the plasmid comprises an EES from a plasmid variant of incompatibility group C (IncC). In one example, the plasmid comprises an EES from a plasmid variant of incompatibility group M (IncM). In one example, the plasmid comprises an EES from a plasmid variant of incompatibility group A (IncA). In another example, one or more of the EES are from plasmid variants of incompatibility group L (IncL) and one or more of the EES are from plasmid variants of incompatibility group C (IncC). In another example, one or more of the EES are from plasmid variants of incompatibility group L (IncL) and one or more of the EES are from plasmid variants of incompatibility group M (IncM). In another example, one or more of the EES are from plasmid variants of incompatibility group C (IncC) and one or more of the EES are from plasmid variants of incompatibility group M (IncM). In another example, one or more of the EES are from plasmid variants of incompatibility group L (IncL), one or more of the EES are from plasmid variants of incompatibility group C (IncC) and one or more of the EES are from plasmid variants of incompatibility group M (IncM).

For example, the recombinant plasmid (e.g., recombinant conjugative plasmid) may comprise one or more (or each) of:
an EES comprising a polynucleotide sequence which is at least 70% identical (e.g., at least 75% identical, or at least 80% identical, or at least 85% identical) to the sequence set forth in SEQ ID NO: 9 (excL variant);
an EES comprising a polynucleotide sequence which is at least 70% identical (e.g., at least 75% identical, or at least 80% identical, or at least 85% identical) to the sequence set forth in SEQ ID NO: 10 (excC variant);
an EES comprising a polynucleotide sequence which is at least 70% identical (e.g., at least 75% identical, or at least 80% identical, or at least 85% identical) to the sequence set forth in SEQ ID NO: 11 (excM variant); and/or
an EES comprising a polynucleotide sequence which is at least 70% identical (e.g., at least 75% identical, or at least 80% identical, or at least 85% identical) to the sequence set forth in SEQ ID NO: 12 (excA variant).

For example, the recombinant conjugative plasmid may comprise one or more (or each) of:
an EES comprising a polynucleotide sequence which is at least 90% identical to the sequence set forth in SEQ ID NO: 9 (excL variant);
an EES comprising a polynucleotide sequence which is at least 90% identical to the sequence set forth in SEQ ID NO: 10 (excC variant);
an EES comprising a polynucleotide sequence which is at least 90% identical to the sequence set forth in SEQ ID NO: 11 (excM variant); and/or
an EES comprising a polynucleotide sequence which is at least 90% identical to the sequence set forth in SEQ ID NO: 12 (excA variant).

For example, the recombinant plasmid (e.g., recombinant conjugative plasmid) may comprise one or more (or each) of:
an EES comprising a polynucleotide sequence which is at least 95% identical (e.g., at least 96% identical, or at least 97% identical, or at least 98% identical, or at least 99% identical) to the sequence set forth in SEQ ID NO: 9 (excL variant);
an EES comprising a polynucleotide sequence which is at least 95% identical (e.g., at least 96% identical, or at least 97% identical, or at least 98% identical, or at least 99% identical) to the sequence set forth in SEQ ID NO: 10 (excC variant);
an EES comprising a polynucleotide sequence which is at least 95% identical (e.g., at least 96% identical, or at least 97% identical, or at least 98% identical, or at least 99% identical) to the sequence set forth in SEQ ID NO: 11 (excM variant); and/or
an EES comprising a polynucleotide sequence which is at least 95% identical (e.g., at least 96% identical, or at least 97% identical, or at least 98% identical, or at least 99% identical)to the sequence set forth in SEQ ID NO: 12 (excA variant).

For example, the recombinant plasmid (e.g., recombinant conjugative plasmid) may comprise one or more (or each) of:
an EES comprising a polynucleotide sequence set forth in SEQ ID NO: 9 (excL variant);
an EES comprising a polynucleotide sequence set forth in SEQ ID NO: 10 (excC variant);
an EES comprising a polynucleotide sequence set forth in SEQ ID NO: 11 (excM variant); and/or
an EES comprising a polynucleotide sequence set forth in SEQ ID NO: 12 (excA variant).

In one example, one or more of the EES are operably-linked to a constitutive promoter. For example, the constitutive promoter may be selected from a promoter that is native to the EES or a strong integron promoter (Pc) or another suitable (including inducible eg ara) promoter.

Alternatively or in addition, one or more of the EES are operably-linked to an inducible promoter. For example, the inducible promoter may be a L-arabinose inducible promoter.

In some examples, each EES is operably-linked to a separate promoter.

In some examples, one or more EES are operably-linked to the same promoter.

In one example, the recombinant plasmid (e.g., recombinant conjugative plasmid) comprises multiple plasmid replication incompatibility (Inc) control regions (inc-control). For example, the Inc control regions are from plasmid variants from an Inc group selected from IncF, IncI, IncA, IncC, IncL, IncM, IncX, IncN, IncH and plasmids with any other known or unknown Inc groups.

In some examples, the Inc control regions and the EES are from the same Inc group(s).

In one example, one or more of the Inc control regions are from plasmid variants of IncF.

In one example, the recombinant plasmid (e.g., recombinant conjugative plasmid) further comprises a selectable marker or gene. For example, the selectable marker or gene may be an antibiotic resistance gene. For example, the selectable marker confers a selectable enzymatic activity.

The present disclosure also provides a bacterial cell comprising a recombinant conjugative plasmid of the disclosure. Preferably the bacteria is a probiotic bacteria. For example, the probiotic bacteria may be selected from a group consisting of the members of Enterobacteriaceae, Lactobacillus, Bifidobacteria, Streptococcus, Lactococcus, Enterococcus, Propionibacterium, Faecalibacterium, Pediococcus, Ruminococcus, Megasphaera and Bacillus.

The present disclosure also provides a composition comprising one or more of viable bacterial cells as described herein.

In one example, the one or more viable bacterial cells are lyophilised. The lyophilised bacterial cells may be microencapsulated and/or provided in a capsule.

In one example, the composition is a food or beverage product. For example, the food or beverage product may be a dairy product (e.g., a yoghurt drink).

The present disclosure also provides a method of preventing or reducing the acquisition and/or spread of a plasmid conferring a trait of interest within population of bacteria, said method comprising introducing to the population one or more bacterial cells of as described herein or a composition comprising same as described herein.

In one example, the trait of interest is an undesirable trait, such as a pathogenic trait conferred by a pathogenic plasmid.

In one example, the trait of interest may be antimicrobial resistance (AMR). In accordance with this example, one or more of the bacterial cells introduced to the bacterial population may comprise a recombinant conjugative plasmid comprising an EES from a plurality of plasmid variants conferring AMR.

In one example, the trait of interest may be bacterial virulence. In accordance with this example, one or more of the bacterial cells introduced to the bacterial population may comprise a recombinant conjugative plasmid comprising an EES from a plurality of plasmid variants conferring bacterial virulence.

In one example, the population of bacteria is a population of gut bacteria and the method comprises administering the one or more bacterial cells or composition comprising same to a subject in need thereof. For example, the method may comprises administering the one or more bacterial cells or composition comprising same to a subject orally.

In one example, the subject is a human.

In one example, the subject is a non-human animal. For example, the non-human animal may be a livestock species or companion animal.

In another example, the population of bacteria to which the probiotic bacterial is introduced is a population of bacteria which colonise plants, and the method comprises contacting the plant or soil in which the plant is growing with the one or more bacterial cells or composition comprising same.

In another example, the population of bacteria to which the probiotic bacterial is introduced is present in an environment selected from a healthcare environment, a soil environment, an environment comprising a water source, an environment comprising waste water, an environment comprising industrial waste, an environment comprising agricultural waste, an environment comprising sewerage and/or an environment comprising biosolids, and the method comprises introducing the one or more bacterial cells or composition comprising same to the environment. For example, the method may be a method of bioremediation.

The present disclosure also provides a food product or beverage comprising one or more bacterial cells as described herein or a composition comprising same.

In one example, the food product or beverage is a dairy product. For example, the food product or beverage may be a yogurt (e.g., a drinkable yogurt).

### BRIEF DESCRIPTION OF THE DRAWINGS

FIG. 1 provides the traS gene sequences for (A) traS_R100 (SEQ ID NO: 4), (B) traS_F (SEQ ID NO: 5) and (C) traS_SLT (SEQ ID NO: 6) which were synthesized for cloning into plasmid backbones. The promoters'-35 and -10 regions are shaded green with underline, RBS sequences are shown in bold-underlined, and coding regions are shaded in grey.

FIG. 2 provides vector maps for probiotic plasmids constructed with a single IncF entry exclusion gene variant: (A) traS_R100, (B) traS_F and (C) traS_SLT.

FIG. 3 provides the synthesized sequence for TraS_3 coding for the three IncF entry exclusion gene variants traS_R100, traS_F and traS_SLT, combined with native promoters for traS_R100, traS_SLT and integron promoter Pc for tras_F and restriction sites (HindIII+XbaI). The promoters'-35 and -10 regions are shaded in green shaded and underline, RBS sequences are shown in bold-underlined, and coding regions are shaded in grey.

FIG. 4 provides a vector map for the probiotic plasmid carrying the three IncF traS variants (traS_3) from FIG. 3.

FIG. 5 provides a vector map for a pBAD33_Gm plasmid backbone comprising the IncF variant traS_R100 under the control of an arabinose inducible promoter.

FIG. 6 provides the synthesized sequence for TraS_3 coding for the three IncF entry exclusion gene variants traS_R100, traS_F and traS_SLT flanked by XbaI+HindIII restriction sites for cloning in the pBAD33-Gm backbone. The promoters'-35 and -10 regions are shaded in green and underlined, RBS sequences are shown in bold-underlined, and coding regions are shaded in grey.

FIG. 7 provides a vector map for a probiotic plasmid having a pBAD33_Gm backbone into which coding sequences for three IncF entry exclusion gene variants traS_R00, traS_F and traS_SLT have been cloned. The IncF EES variants traS_R100 and traS_SLT are each under control of arabinose inducible promoters, whereas the IncF EES variant traS_F is under the control of its native promoter.

FIG. 8 provides a summary of the results of conjugation experiments performed with IncF plasmids from a donor J53 to a recipient BW25113Rf with or without different probiotic plasmid constructs of the disclosure.

FIG. 9 provides alignments of different variants of exclusion proteins present in IncA, C, IncM and IncL plasmid sequences found in the GenBank. (A) All the IncC plasmids in the GenBank have only one Exc protein type. Exc proteins of IncC and IncA fall into the same exclusion group and prevent each other's entry. (B) and (C) shows the exclusion protein variants from IncL and IncM plasmids.

FIG. 10 provides the nucleotide sequence of the exc(L)_exc(C)_fosA3 gBlock. The underlined stretches of nucleotides from 5′-3′ are the coding region of IncL_exc, IncC_exc and fosA3 genes, respectively.

FIG. 11 provides a physical map of the conjugative probiotic plasmid PB1.1 (Size: 62702 bp). Two orange regions are for two independent insertion locations.

FIG. 12 provides the nucleotide sequence of the probiotic plasmid PB.1.1.

FIG. 13 provides a summary of the results of conjugation experiments performed with IncM, IncC or IncL plasmids with or without a probiotic plasmid construct PB1.1 of the disclosure. Blue bars are for the transfer of different AMR plasmids into the empty J53Az bacteria during liquid mating experiments. Orange bars are for the transfer of the respective plasmids into the J53Az carrying conjugative probiotic plasmid PB1.1. Transfer of the AMR plasmids is reduced significantly when recipient bacteria have probiotic plasmids in them. Conjugation frequency is the ratio of transconjugants per recipient. Data shown is the mean of three independent liquid mating experiments with standard errors.

FIG. 14 provides a summary of the results of mouse experiments demonstrating the acquisition of different AMR plasmids with or without a probiotic plasmid construct PB1.1 of the disclosure. AMR colonised mice were co-housed (8 hours each day) with control mice receiving no probiotic plasmid treatment (none) or with probiotic plasmid-treated (probiotic) mice. AMR plasmids were detected in exposed mice from the non-treatment group (none) at different timepoints. No AMR plasmid was detected in mice that received probiotic plasmids (probiotic). Different coloured circles represent different mice in each group.

FIG. 15 provides the summary of results of antibiotic treatment 120 h post exposure to AMR colonised mice. Probiotic plasmid colonised and non-colonised control mice were provided CTX in sucrose water for 2 days after 120 h of exposure to AMR plasmid colonised mice. CTX-resistant bacterial colonisation was greatly enhanced in mice whose gut flora was not colonised with probiotic plasmid (None), but detected in only 2 of 9 mice, at a very low level in the probiotic plasmid colonised mice (probiotic). Different coloured circles represent different mice in the group.

### KEY TO THE SEQUENCE LISTING

SEQ ID NO: 1 provides a polynucleotide sequence for the F plasmid variant entry exclusion system designated traS_R100.
SEQ ID NO: 2 provides a polynucleotide sequence for the F plasmid variant entry exclusion system designated traS_F.
SEQ ID NO: 3 provides a polynucleotide sequence for the F plasmid variant entry exclusion system designated traS_SLT SEQ ID NO: 4 provides a polynucleotide sequence for the F plasmid variant entry exclusion system designated traS_R100 with native promoter, RBS and restriction sites.
SEQ ID NO: 5 provides a polynucleotide sequence for the F plasmid variant entry exclusion system designated traS_F with native promoter, RBS and restriction sites SEQ ID NO: 6 provides a polynucleotide sequence for the F plasmid variant entry exclusion system designated traS_SLT with native promoter, RBS and restriction sites SEQ ID NO: 7 provides a polynucleotide sequence for a construct comprising the three F plasmid variant entry exclusion systems designated traS_R100, traS_SLT with native promoter, and traS_F with integron promoter Pc, RBS and flanking HindIII and XbaI restriction sites.
SEQ ID NO: 8 provides a polynucleotide sequence for a construct comprising (i) a consensus RBS with the traS_R100 variant polynucleotide sequence, (ii) the traS_F variant polynucleotide sequence with its native promoter and RBS, and (iii) the traS_SLT variant polynucleotide sequence with an araBAD promoter and consensus RBS, with HindIII and XbaI restriction sites flanking the three traS variant sequences.
SEQ ID NO: 9 provides a polynucleotide sequence for the C plasmid variant entry exclusion system designated ExcC.
SEQ ID NO: 10 provides a polynucleotide sequence for the L plasmid variant entry exclusion system designated ExcL.
SEQ ID NO: 11 provides a polynucleotide sequence for the M plasmid variant entry exclusion system designated ExcM.
SEQ ID NO: 12 provides a polynucleotide sequence for the A plasmid variant entry exclusion system designated ExcA.
SEQ ID NO: 13 provides the nucleotide sequence of the exc(L)_exc(C)_fosA3 gBlock SEQ ID NO: 14 provides a polynucleotide sequence for probiotic plasmid PB1.1.

### DETAILED DESCRIPTION

### General Techniques

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

The words “a” and “an” when used in this disclosure, including the claims, denotes “one or more.”

As used herein, the terms “about” and “approximately” are interchangeable, and should generally be understood to refer to a range of numbers around a given number, as well as to all numbers in a recited range of numbers. Moreover, all numerical ranges herein should be understood to include each whole integer within the range.

The terms “e.g.,” and “i.e.” as used herein, is used merely by way of example, without limitation intended, and should not be construed as referring only those items explicitly enumerated in the disclosure.

### Plasmids

The present disclosure provides recombinant conjugative plasmids comprising a plurality of entry exclusion systems (EES) from a plurality of plasmid variants, where the plasmid variants from which the EESs are derived comprise one or more genes conferring a trait of interest e.g., typically a trait which is undesirable in a bacterial population. Exemplary traits which are undesirable in bacterial populations include, but are not limited to, antimicrobial resistance (AMR) and bacterial virulence. In some examples, the EES are derived from pathogenic plasmid variants. As used herein, the term “pathogenic” is understood in the art, for example, as causing pathogenicity such as morbidity and/or mortality of an organism or population of organisms. In general, pathogenic plasmids often carry resistance genes against antibiotics or heavy metals, genes for the metabolism of atypical substrates or genes for a number of species-specific characteristics, such as metabolic properties or virulence factors. Thus, recombinant conjugative plasmids of the disclosure may comprise multiple EES from pathogenic plasmid variants harbouring genes which confer any one or more of these undesirable traits.

The plasmids of the disclosure exploit the properties of EES to inhibit the transfer of plasmids to recipient bacteria carrying identical or closely-related plasmids. The EES, specifically, is present in most known conjugative plasmid types and can inhibit the acquisition of the closely-related plasmid in a recipient bacteria by 10 to 10,0000 fold, depending on the plasmid type. The present inventors have discovered that a recombinant plasmid which comprises EES from multiple plasmid variants can be used prevent or reduce the acquisition and/or spread within a bacterial population of a range of closely-related plasmids variants (e.g., such as pathogenic plasmid variants) which possess the same or related EES.

As used herein, a “plasmid” will be understood to mean a circular, double-stranded DNA molecule which forms an extrachromosomal self-replicating genetic element that can be used as a vehicle for introducing a nucleic acid into bacterial and eukaryotic cells. Bacterial plasmids are usually circularly covalently closed and supercoiled. In some examples, a plasmid may be transmitted from one bacterium to another (including other species of bacterium) through a process known as “conjugation”. This host-to-host transfer of genetic material is one mechanism of horizontal gene transfer. A “conjugative plasmid” as used herein will therefore be understood to be a plasmid which is capable of host-to-host or horizontal transfer.

The term “recombinant”, as used in the context of a plasmid of the disclosure, shall be understood to mean an artificial or synthetic plasmid made using a plasmid backbone and components from one or more plasmids capable of expressing one or more DNA expression cassettes or operons. A skilled person will appreciate that any number of plasmid backbones could be used for constructing the recombinant plasmid of the disclosure. However, exemplary plasmid backbones include pBCSK+, pBAD33_Gm, pJIBE401, and pJIMKCore_M as described herein. Such backbones may be modified to carry inter alia genes coding for replication and partitioning systems, as well as multiple entry exclusion systems.

However, a skilled person will also appreciate that construct comprising a plurality of entry exclusion systems (EES) from a plurality of plasmid variants in accordance with the present disclosure may also be delivered to a host bacteria by other recombinant means, such as, for example, episomal phages (pseudolysogeny) or transposable genetic elements or other integrative and conjugative elements. In this regard, whilst the use of plasmids to introduce multiple EES to a host bacteria as described herein is a particularly preferred embodiment, a skilled person will appreciate that the concept may be extended to other delivery means and that these alternative embodiments are contemplated herein. Accordingly, in other examples, the present disclosure also provides an episomal phage, transposable genetic element or other integrative and conjugative element comprising a plurality of EES from a plurality of plasmid variants, where the plasmid variants from which the EESs are derived comprise one or more genes conferring a trait of interest e.g., typically a trait which is undesirable in a bacterial population. Exemplary traits which are undesirable in bacterial populations include, but are not limited to, AMR and bacterial virulence.

As used herein, an “entry exclusion system” or “EES” will be understood to mean a genetic element or system which permits entry exclusion. “Entry exclusion” denotes a property of plasmids by which the cells that contain them become poor recipients to similar plasmids during additional conjugation rounds. A plasmid's entry exclusion system operates by inhibiting physical entry of an incoming plasmid into a cell where that incoming plasmid exhibits the exclusion phenotype. The inclusion of an entry exclusion system frees a plasmid from competition with related plasmids at segregation during bacterial division. Thus, entry exclusion (i) prevents incompatible incoming plasmids from eliminating a pre-existing (e.g. less undesirable but closely related) plasmid within the host cell, (ii) avoids uneconomical excess of DNA transfer and (iii) averts death of the recipient cell by lethal zygosis.

Entry exclusion proteins from diverse sources are not equal in effectiveness, but exhibit variable exclusion indices that can be increased by over-expressing the exclusion protein. As used herein, the term “exclusion index” refers to the transfer frequency of a given plasmid to a plasmid-free recipient divided by the frequency of transfer to a recipient carrying the same plasmid or plasmid with a similar entry exclusion system. For instance, plasmid F showed an EI of 100-300 in mating between Escherichia coli since it transferred 100-300 times better to a plasmid-free recipient than to an F+ recipient (Achtman et al., 1977; Achtman and Skurray, 1977). In a preferred example, the exclusion index or protection index is the ratio of conjugation frequency to the empty recipient (without exclusion system) and to the recipient bacteria with a probiotic plasmid (with cloned exclusion gene/s). The exclusion index indicates the fold of plasmid transfer inhibition. Thus, the higher exclusion index value indicates stronger protection from the acquisition of plasmid tested.

As described herein, the recombinant conjugative plasmid (or other vehicle for delivery of DNA constructs as described herein) comprises plurality of EES from a plurality of plasmid variants. In one example, the recombinant conjugative plasmid comprises EES from two plasmid variants comprising a gene conferring the trait of interest. In one example, the recombinant conjugative plasmid comprises EES from at least three (e.g., 3, or 4, or 5, or 6, or 7, or 8, or 9, or 10 or more) plasmid variants comprising a gene conferring the trait of interest. In some examples, the two or more of the EES within the recombinant conjugative plasmid are from the same incompatibility group. In some examples, all of the EES within the recombinant conjugative plasmid are from the same incompatibility group. In other examples, the EES within the recombinant conjugative plasmid are from different incompatibility groups (e.g., the EES may be derived from 2, 3, 4 or more different incompatibility groups).

Incompatibility grouping represents the inability of two plasmids to coexist stably over a number of generations in the same bacterial cell line. Plasmid which are incompatible with one another are assigned to the same “incompatibility group” or “Inc”. Conversely, plasmids which are categorised in different incompatibility groups may be able to co-exist in the same bacterial cell. “Plasmid incompatibility” therefore refers to the inability of plasmids to co-exist, stably, within the same cell when they have similar or identical systems for plasmid replication and/or plasmid partition, i.e. the segregation of each plasmid into daughter cells during cell division along with entry exclusion genes. Two incompatible plasmids, which occupy the same cell would, in the absence of a selective pressure for both plasmids, tend to segregate or partition to different cells during cell division. The stable intracellular co-existence of one plasmid with another requires that each plasmid is able to control, independently of the other, its own replication/partition such that it can establish and maintain a stable copy number. However, the inability of a given plasmid to maintain a stable copy number in the presence of another plasmid is the characteristic feature of incompatibility. Competition for cell resources can result when two plasmids of the same incompatibility group are found in the same cell. Whichever plasmid is able to replicate faster, or provides some other advantage, will typically be represented to a disproportionate degree among the copies allowed by the incompatibility system. Surprisingly, plasmids can also be incompatible when they both possess the same functions for partitioning themselves into daughter cells.

Plasmids typically fall into only one of the many existing incompatibility groups. There are more than 30 known incompatibility groups. Examples include, but are not limited to; IncN, IncW, IncL, IncM, IncT, IncU, IncW, IncY, IncB/O, IncIl, IncK, IncCom9, IncFI, IncFII, IncFIII, IncHI1, IncHI2, IncX, IncA, IncC, IncD, IncFIV, IncFV/FO, IncFVI, IncH1 3, Incl2, IncI, IncJ, IncV, IncP, IncQ, and the like, including variants thereof. Accordingly, in some examples, one or more of the EES in the recombinant plasmid is derived from a plasmid variant of an incompatibility group (Inc) selected from those described hereinabove. In some examples, one or more (or all) of the EES in the recombinant plasmid are from a plasmid variant of an incompatibility group (Inc) selected from IncF, IncI, IncA, IncC, IncM, IncL, IncN, IncX, IncP and IncH.

In one particular example, one or more of the EES are from a plasmid variant of IncF. In another example, all of the EES are from a plasmid variant of IncF. For example, the recombinant conjugative plasmid may comprise EES from at least 2 (e.g., at least 3, or 4, or 5, or 6, or 7, or 8, or 9, or 10 or more) plasmid variants of IncF.

As described herein, in some examples the entry exclusion systems may be derived from plasmid variants of incompatibility group F, which is the largest group in the AMR plasmids. Due to their considerable variations in their replication systems/genes, it has been a challenge up until this point to cure, and/or protect against, all plasmids within IncF using existing plasmid-based approaches (See e.g., Kamruzzaman M et al., 2017 and Bikard et al., 2014). The F plasmid exerts exclusion using two different EES genes, traT and traS. The majority of F plasmid exclusion activity is thought to be attributable to TraS (EI of around 200, versus 20 for TraT). The EI of F plasmid EES is also thought to be gene dosage dependent since. In this regard, Skurray et al., and Achtman et al. (Skurray, Willetts et al. 1976, Achtman, Kennedy et al. 1977) showed that when traS and traT were cloned in a multicopy plasmid, the EI increased to 10,000. In one example, one or more of the EES in the recombinant conjugative plasmid are traS gene variants.

The present disclosure provides three genetic variants of the entry exclusion gene traS from the F plasmid, designated traS-R100, traS_F and traS_SLT, which collectively represent most IncF plasmid variation.

In one example, the recombinant conjugative plasmid of the disclosure comprises an EES comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 1 (the “traS_F variant EES”). For example, the recombinant conjugative plasmid of the disclosure may comprise an EES comprising a polynucleotide sequence set forth in SEQ ID NO: 1.

In one example, the recombinant conjugative plasmid of the disclosure comprises an EES comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 2 (the “traS_R100 variant EES”). For example, the recombinant conjugative plasmid of the disclosure may comprise an EES comprising a polynucleotide sequence set forth in SEQ ID NO: 2.

In one example, the recombinant conjugative plasmid of the disclosure comprises an EES comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 3 (the “traS_SLT variant EES”). For example, the recombinant conjugative plasmid of the disclosure may comprise an EES comprising a polynucleotide sequence set forth in SEQ ID NO: 3.

In some examples, the traS_F variant EES, the traS_R100 variant EES and the traS_SLT variant EES are combined in the recombinant conjugative plasmid of the disclosure.

A skilled person will appreciate that the recombinant conjugative plasmid comprising the F plasmid variant EES genes described herein may further comprise EES from other incompatibility groups of bacteria (e.g., bacteria known to be pathogenic). Exemplary additional EES genes from other incompatibility groups of bacteria include but are not limited to trbK (IncPα plasmids), eex (IncN plasmid pKM101 and IncW plasmid R388), exc (IncI plasmid R144), eexA and eexB (IncHI1 plasmid R27), eexC (IncC plasmids), mbeD (ColEI-like plasmids—not self-transmissible), sec10/prgA (pAD1 and other sex pheromone plasmids), pif (pSAM2 plasmid), and their homologs, orthologs and variants thereof.

Alternatively, or in addition, the recombinant conjugative plasmid of the disclosure may comprise one or more (e.g., 2, 3 or 4 or more) EES from a plasmid variant of incompatibility group L (IncL), incompatibility group C (IncC), incompatibility group M (IncM) and/or incompatibility group A (IncA). This may an alternative or in addition to the EES from a plasmid variant of incompatibility group F as described in the foregoing examples.

In one particular example, the recombinant conjugative plasmid of the disclosure comprises an EES from a plasmid variant of incompatibility group L (IncL) comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 9. For example, the recombinant conjugative plasmid of the disclosure may comprise an EES comprising a polynucleotide sequence set forth in SEQ ID NO: 9.

In one particular example, the recombinant conjugative plasmid of the disclosure comprises an EES from a plasmid variant of incompatibility group C (IncC) comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 10. For example, the recombinant conjugative plasmid of the disclosure may comprise an EES comprising a polynucleotide sequence set forth in SEQ ID NO: 10.

In one particular example, the recombinant conjugative plasmid of the disclosure comprises an EES from a plasmid variant of incompatibility group M (IncM) comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 11. For example, the recombinant conjugative plasmid of the disclosure may comprise an EES comprising a polynucleotide sequence set forth in SEQ ID NO: 11.

In one particular example, the recombinant conjugative plasmid of the disclosure comprises an EES from a plasmid variant of incompatibility group A (IncA) comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 12. For example, the recombinant conjugative plasmid of the disclosure may comprise an EES comprising a polynucleotide sequence set forth in SEQ ID NO: 12.

In some examples, the recombinant conjugative plasmid of the disclosure comprises an EES from a plasmid variant of IncL as described herein and an EES from a plasmid variant of IncC as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein and an EES from a plasmid variant of IncM as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein and an EES from a plasmid variant of IncA as described herein. The recombinant conjugative plasmid may further comprise an EES from a plasmid variant of IncF as described herein.

In some examples, the recombinant conjugative plasmid of the disclosure comprises an EES from a plasmid variant of IncC as described herein and an EES from a plasmid variant of IncM as described herein. The recombinant conjugative plasmid may further comprise an EES from a plasmid variant of IncF as described herein.

In some examples, the recombinant conjugative plasmid of the disclosure comprises an EES from a plasmid variant of IncA as described herein and an EES from a plasmid variant of IncM as described herein. The recombinant conjugative plasmid may further comprise an EES from a plasmid variant of IncF as described herein.

In some examples, the recombinant conjugative plasmid of the disclosure comprises an EES from a plasmid variant of IncA as described herein and an EES from a plasmid variant of IncC as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein, an EES from a plasmid variant of IncC as described herein, and an EES from a plasmid variant of IncA as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein, an EES from a plasmid variant of IncM as described herein, and an EES from a plasmid variant of IncA as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein, an EES from a plasmid variant of IncC as described herein, and an EES from a plasmid variant of IncM as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein, an EES from a plasmid variant of IncA as described herein, and an EES from a plasmid variant of IncC as described herein. The recombinant conjugative plasmid may further comprise an EES from a plasmid variant of IncF as described herein.

In some examples, the recombinant conjugative plasmid of the disclosure comprises an EES from a plasmid variant of IncC as described herein, an EES from a plasmid variant of IncA as described herein, and an EES from a plasmid variant of IncM as described herein. The recombinant conjugative plasmid may further comprise an EES from a plasmid variant of IncF as described herein.

In some examples, the recombinant conjugative plasmid of the disclosure comprises an EES from a plasmid variant of IncL as described herein, an EES from a plasmid variant of IncC as described herein, an EES from a plasmid variant of IncM as described herein and an EES from a plasmid variant of IncA as described herein. The recombinant conjugative plasmid may further comprise an EES from a plasmid variant of IncF as described herein.

In some examples, the recombinant conjugative plasmid of the disclosure comprises an EES from a plasmid variant of IncF as described herein, an EES from a plasmid variant of IncL as described herein, an EES from a plasmid variant of IncC as described herein, an EES from a plasmid variant of IncM as described herein and an EES from a plasmid variant of IncA as described herein.

Alternatively, or in addition, one or more of the EES included in the recombinant conjugative plasmid of the disclosure may be derived from a multi-drug resistant organism or pathogen including, but not limited to, organisms belonging to the genus Acinetobacter, Citrobacter, Enterobacter, Enteroccus, Escherichia, Klebsiella, Serratia or Staphylococcus. Exemplary multi-drug resistant organisms include Acinetobacter baumannii such as ATCC isolate #2894233-696-101-1, ATCC isolate #2894257-696-101-1 ATCC isolate #2894255-696-101-1, ATCC isolate #2894253-696-101-1, or ATCC #2894254-696-101-1; Citrobacter freundii such as ATCC isolate #33128, ATCC isolate #2894218-696-101-1, ATCC isolate #2894219-696-101-1, ATCC isolate #2894224-696-101-1, ATCC isolate #2894218-632-101-1, or ATCC isolate #2894218-659-101-1; Enterobacter cloacae such as ATCC isolate #22894251-659-101-1, ATCC isolate #22894264-659-101-1, ATCC isolate #22894246-659-101-1, ATCC isolate #22894243-659-101-1, or ATCC isolate #22894245-659-101-1; Enteroccus faecalis such as ATCC isolate #22894228-659-101-1 ATCC isolate #22894222-659-101-1, ATCC isolate #22894221-659-101-1, ATCC isolate #22894225-659-101-1, or ATCC isolate #22894245-659-101-1; Enteroccus faecium such as ATCC isolate #51858, ATCC isolate #35667, ATCC isolate #2954833_2694008 ATCC isolate #2954833_2692765, or ATCC isolate #2954836_2694361; Escherichia coli such as ATCC isolate CGUC 11332, CGUC 11350, CGUC 11371, CGUC 11378, or CGUC 11393; Klebsiella pneumonia such as ATTC isolate #27736, ATTC isolate #29011, ATTC isolate #20013, ATTC isolate #33495, or ATTC isolate #35657; Serratia marcescens such as ATCC isolate #43862, ATCC isolate #2338870, ATCC isolate #2426026, ATCC isolate #SIID 2895511, or ATCC isolate #SIID 2895538; or Staphylococcus aureus such as ATCC isolate #JHH 02, ATCC isolate #JHH 02, ATCC isolate #JHH 03, ATCC isolate #JHH 04, ATCC isolate #JHH 05, or ATCC isolate #JHH 06.

Alternatively, or in addition, one or more of the EES included in the recombinant plasmid of the disclosure may be derived from a virulence plasmid harbouring a virulence genes that encode a virulence factor/virulence determinant. For example, pathogenic E. coli, dysentery-causing Shigella and other enteric bacteria, such as Salmonella typhi (typhoid) and Y. pestis (bubonic plague), generally rely on plasmid-borne virulence factors. Accordingly, one or more of the EES included in the recombinant conjugative plasmid of the disclosure may be derived from a virulence plasmid.

The EES genes/variants may each be linked to a promoter to drive their expression in the host cell. The term “promoter” as used herein shall be understood to define a regulatory DNA sequence, generally located upstream of a gene or sequence to be expressed, that mediates the initiation of transcription by directing RNA polymerase to bind to DNA and initiating RNA synthesis.

A promoter for inclusion in a recombinant plasmid of the disclosure can be an endogenous promoter, a heterologous promoter or a combination thereof.

In some examples, the promoter is a constitutive promoter (e.g., a T7, SP6, T3, integron (Pc) or other suitable constitutive promoter).

In other examples, one or more of the EES genes may be under the control of an inducible promoter. An inducible promoter may be a nucleic acid sequence or an operon system that directs the conditional expression of the EES gene in the presence of a certain compound, nutrient, amino acid, sugar, peptide, protein or condition (e.g., light, oxygen, heat, cold). Alternatively, the inducible promoter may comprise one or more repressor elements such that the absence of a certain compound, nutrient, amino acid, sugar, peptide, protein or condition is required to induce transcription of the EES gene. Any suitable inducible promoter, system or operon known in the art may be used. Non-limiting examples of inducible promoters which are contemplated include lactose regulated systems (e.g., lactose operon systems), sugar regulated systems, metal regulated systems, steroid regulated systems, alcohol regulated systems, IPTG inducible systems, arabinose regulated systems (e.g., arabinose operon systems, e.g., an ARA operon promoter, pBAD, pARA, PARAE, ARAE, ARAR-ParaE, portions thereof, combinations thereof and the like), synthetic amino acid regulated systems (e.g., see Rovner A J, et al., (2015) Nature 518(7537):89-93), fructose repressors, a tac promoter/operator (pTac), tryptophan promoters, PhoA promoters, recA promoters, proU promoters, cst-1 promoters, tetA promoters, cadA promoters, nar promoters, PL promoters, cspA promoters, the like or combinations thereof. In one particular example, the inducible promoter is a L-arabinose inducible promoter.

In some examples, two or more, or all, of the EES genes within the recombinant conjugative plasmid are operably linked to the same promoter with the plasmid i.e., a single promoter driving expression of the plurality of EES gene variants.

In other examples, each EES gene variant is operably-linked to a separate promoter.

In some examples, the entry exclusion genes are linked to their native promoters and ribosome binding sites (RBS). For example, the recombinant conjugative plasmid may comprise the traS_F variant EES, the traS_R100 variant EES and the traS_SLT variant EES, each operably linked to its native promoter and RBS. For example, the recombinant conjugative plasmid of the disclosure may comprise the polynucleotide sequences set forth in SEQ ID NO: 4-6 or sequences which are at least 70% (e.g., at least 75%, or at least 80%, or at least 85%, or at least 90%, or at least 95%, or at least 96%, or at least 97%, or at least 98%, or at least 99%) identical to the sequences set forth in SEQ ID NOs: 4-6.

In some examples, the recombinant plasmid of the disclosure may comprise a toxin-antitoxin system or addition system to prevent plasmid-free segregants from surviving. As used herein, the terms “toxin-antitoxin system”, “addiction system” and similar terms (e.g., killer system, killing-anti-killing, post-segregational killing, poison-antidote, plasmid addiction system or programmed cell death) are used to describe genetic systems which have evolved in plasmids to ensure that a host cell is selectively killed if it has not received any copy of the plasmid. The molecular basis of the “toxin-antitoxin system” requires the existence of at least two genes: one specifying a stable toxic agent i.e., the “toxin”, and another coding for an unstable factor which prevents lethal action of the gene encoding the toxic agent i.e., the “antitoxin”. The toxin and antitoxin genes may each be operably linked to a promoter within the plasmid to drive expression of the respective genes. Toxin-antitoxin systems for inclusion in bacterial plasmids are known in the art and contemplated herein.

Most plasmids are capable of replicating independently. In order for plasmids to replicate independently within a cell (e.g., a bacterial cell), they must possess a stretch of DNA that can act as an “origin of replication”. Plasmids that are capable of replicating autonomously within a host cell are also referred to as replicons. According to an example in which the recombinant conjugative plasmid of the disclosure is capable of replicating autonomously, the plasmid will comprise an origin of replication.

Other plasmids can be stably integrated into the genome of a host cell, and are thereby replicated along with the host genome.

The recombinant conjugative plasmid may further comprise one or more plasmid replication incompatibility (Inc) control regions (inc-control). In one particular example, the one or more inc-control regions is/are from one or more aforementioned plasmid incompatibility groups. For example, the one or more inc-control regions is/are from a plasmid incompatibility group selected from IncF, IncI, IncA, IncC, IncL, IncM, IncX, IncP, IncN and IncH. However, a skilled person will appreciate that the inc-control regions may be from any known, or yet to be identified, plasmid incompatibility group.

A plasmid of the disclosure may also comprise a number of other genetic elements, such as a gene for plasmid-specific replication initiation protein (Rep), repeating units called iterons, DnaA boxes, and an adjacent AT-rich region. In addition, the plasmid may include one or more nucleic acid segments, genes, promoters, enhancers, activators, multiple cloning regions, or any combination thereof, including segments that are obtained from or derived from one or more natural and/or artificial sources.

The recombinant conjugative plasmid of the disclosure may further comprise one or more selectable markers or genes to enable selection of cells (e.g., bacterial cells) comprising the plasmid. Selectable markers genes suitable for inclusion in a plasmid will be known to a person of skill to the art. In one example, the selectable marker may be an antibiotic resistance gene. Antibiotic resistance genes confer resistance to antibiotic compounds, such as ampicillin, kanamycin, chloramphenicol, tetracycline, rifampicin, neomycin, hygromycin, erythromycin, methotrexate, and gentamycin, for example. In another example, the selectable marker is a gene which confers a selectable enzymatic activity, such as beta-galactosidase, or the lactose operon.

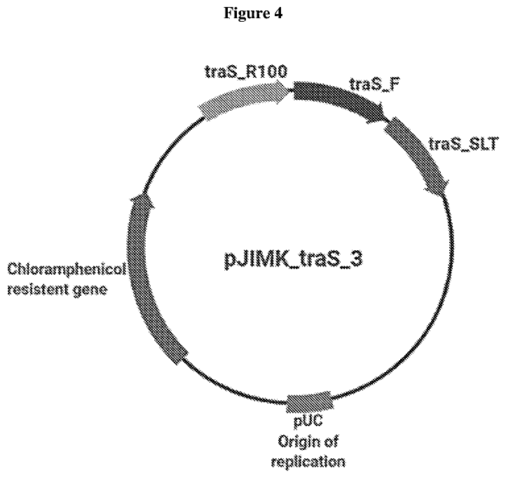

In one example, a recombinant plasmid of the disclosure may comprise the entry exclusion gene variants designated traS_F, traS_R100 and traS_SLT as described herein (by way of reference to SEQ ID NO: 1-3) operably linked to promoter and RBS sequences with flanking HindIII and XbaI restriction sites within a pBCSK+ backbone (i.e., pJIMK_traS_3, FIG. 4). For example, the recombinant plasmid of the disclosure may comprise the sequence set forth in SEQ ID NO. 7.

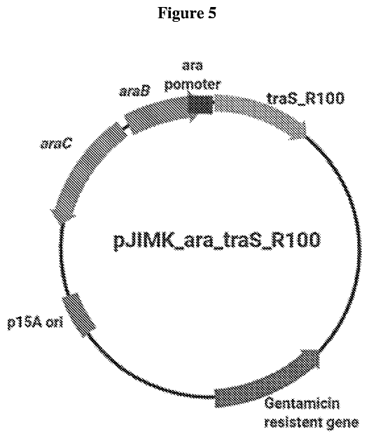

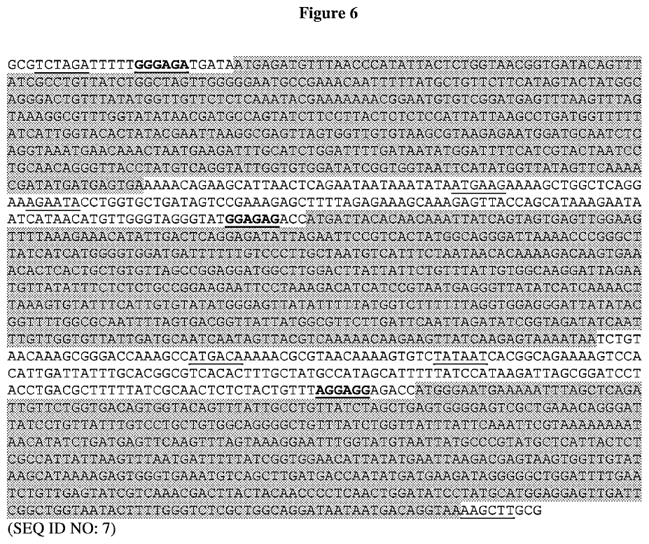

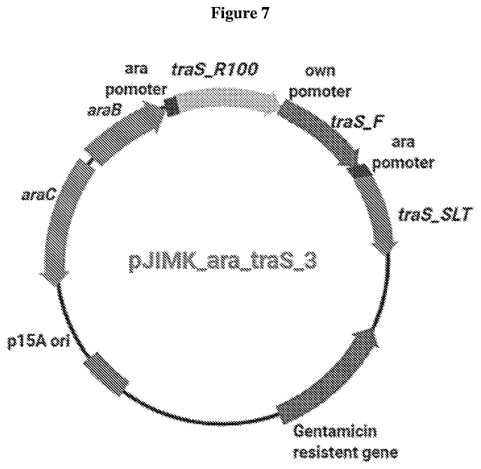

In one example, a recombinant plasmid of the disclosure may comprise the entry exclusion gene variants designated traS_F, traS_R100 and traS_SLT as described herein (by way of reference to SEQ ID NO: 1-3), wherein at least one of the traS variants is operably linked to a pBAD arabinose operon promoter as set forth in SEQ ID NO. 8 (FIG. 6). For example, the traS variants may be combined within a pBAD33_Gm backbone (e.g., pJIMK_ara_traS_R100, FIG. 5). For example, a construct comprising the traS variants may be cloned into the HindIII+XbaI sites of a pBAB33_Gm backbone to produce pJIMK_ara_traS_3, wherein the IncF EES variants traS_R100 and traS_SLT are each under control of arabinose inducible promoters, and the IncF EES variant traS_F is under the control of its native promoter (FIG. 7).

In one example, a recombinant plasmid of the disclosure may comprise the entry exclusion gene variants designated IncC, IncL and IncM as described herein (by way of reference to SEQ ID NO: 9-11 respectively) operably linked to promoter and RBS sequences with any suitable restriction sites within a cloning vector e.g., pBCSK+ backbone. For example, the recombinant plasmid of the disclosure may comprise the sequences set forth in SEQ ID NOs: 9-11. Alternatively, the recombinant plasmid of the disclosure may comprise the sequences set forth in SEQ ID NO 11 and 13.

In one example, a recombinant plasmid of the disclosure may comprise the entry exclusion gene variants designated IncC, IncL, IncM, and IncA as described herein (by way of reference to SEQ ID NO: 9-12 respectively) operably linked to promoter and RBS sequences with any suitable restriction sites within a cloning vector e.g., pBCSK+ backbone. For example, the recombinant plasmid of the disclosure may comprise the sequences set forth in SEQ ID NOs: 9-12. Alternatively, the recombinant plasmid of the disclosure may comprise the sequences set forth in SEQ ID NO 11 and 13.

### Probiotic Bacteria

The present disclosure also provides bacterial cells comprising the recombinant conjugative plasmids as described herein. That is, bacterial cells into which the recombinant conjugative plasmids of the disclosure have been introduced or progeny of such bacterial cells. Such bacterial cells may be used as probiotics to prevent or reduce the acquisition and/or spread of plasmids (such as pathogenic plasmids) expressing incompatible entry exclusion system genes within bacterial populations. Accordingly, the bacteria as described herein may be a probiotic bacteria.

The terms “probiotic”, “probiotics” or similar, as used in connection with bacteria of the disclosure, shall be understood to mean bacteria that enhance the growth and/or health of beneficial bacterium in a particular environment (e.g., the gastrointestinal tract of a subject, soil or water system). Alternatively, or in addition, “probiotic”, “probiotics” or similar, may assist in diminishing the growth and/or prevalence of pathogenic bacterium in a particular environment (e.g., the gastrointestinal tract, soil or healthcare environment). For example, a probiotic bacterium that is administered to a subject in an adequate amount should confer a health benefit to the subject (e.g., a human or animal host), such as by improving gastrointestinal microbial balance. In another example, a probiotic bacterium which is applied or otherwise introduced to soil, wastewater, biosolid or other environment in an adequate amount may improve the microbial balance in that environment and result in one or more beneficial outcomes.

Probiotic bacteria of the present disclosure are typically non-pathogenic and may demonstrate (i) one or more beneficial functions within the gastrointestinal tract of human and non-human animals or (ii) one or more beneficial qualities which make them suitable in bioremediation application.

Exemplary probiotic, non-pathogenic bacteria include Escherichia coli, non-pathogenic members of the Enterobacteriaceae species, and other Bacillus species include, but are not limited to: Bacillus coagulans; Bacillus coagulans Hammer; and Bacillus brevis subspecies coagulans, Bacillus laevolacticus, Bacillus subtilis, Bacillus uniflagellatus, Bacillus laterosporus, Bacillus laterosporus BOD, Bacillus megaterium, Bacillus polymyxa, Bacillus licheniformis, Bacillus pumilus, and Bacillus stearothermophilus. Exemplary probiotic Lactobacillus species include, but are not limited to: Lactobacillus acidophilus, Lactobacillus casei, Lactobacillus paracasei, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus delbruekii, Lactobacillus, bulgaricus, Lactobacillus salivarius, Lactobacillus plantarum, Lactobacillus fermentum, Lactocobacillus helveticus, and Lactobacillus sporogenes. Exemplary probiotic Sporolactobacillus species include all Sporolactobacillus species, for example, Sporolactobacillus P44. Exemplary probiotic Bifidobacterium species include, but are not limited to: Bifidobacterium adolescentis, Bifidobacterium animalis, Bifidobacterium adolescentis Bifidobacterium bifidum, Bifidobacterium bifidum, Bifidobacterium breve, Bifidobacterium infantis, Bifidobacterium infantis, Bifidobacterium longum, and any genetic variants thereof. Other strains that could be employed due to probiotic activity include members of the Lactococcus such as Lactococcus lactis, Lactococcus diacetylactis, Lactococcus cremoris, and Streptococcus (Enterococcus) genus. For example, Enterococcus faecium, is commonly used as a livestock probiotic and, thus, could be utilized as a co-administration agent. Similarly, Ruminococcus sp and Megasphaera could be utilised in applications where the bacteria is to be administered to a livestock species as a probiotic.

For example, the probiotic bacteria may be selected from a group consisting of Escherichia, Klebsiella, Citrobacter, Lactobacillus, Bifidobacteria, Streptococcus, Lactococcus, Enterococcus, Propionibacterium, Faecalibacterium, Pediococcus, Ruminococcus, Megasphaera and Bacillus.

Non-pathogenic E. coli exemplary bacterium which may be used as a probiotic in the present disclosure since it is capable of colonization in the highly acidic environment of the gastrointestinal tract, particularly the human gastrointestinal tract.

Transformation of a bacterial cell with the recombinant conjugative plasmid of the disclosure may, for instance, be effected by any means known in the art, including, but not limited to, protoplast transformation (see, e.g., Chang and Cohen, 1979, Molecular General Genetics 168: 111-115), by using competent cells (see, e.g., Young and Spizizin, 1961, Journal of Bacteriology 81: 823-829, or Dubnar and Davidoff-Abelson, 1971, Journal of Molecular Biology 56:209-221), by electroporation (see, e.g., Shigekawa and Dower, 1988, Biotechniques 6: 742-751), or by conjugation (see, e.g., Koehler and Thorne, 1987, Journal of Bacteriology 169: 5771-5278).

The growth of these various probiotic bacterial species to form cell cultures, cell pastes, and spore preparations is generally well-known within the art. A skilled person will be able to determine appropriate growth/culture conditions depending on whether the bacteria is an aerobe, a facultative anaerobe or an obligate anaerobes. Such culture methods are known in the art.

In some examples, the probiotic bacteria are encapsulated e.g., to protect the bacteria and maintain viability. Protection of the bacteria is achieved if either a majority of cells is still viable or is still metabolically active or if more of the encapsulated cells remain viable when compared with unencapsulated cells which are treated under the same conditions. Methods of encapsulating (e.g., microencapsulation) of bacteria are known in the art.

Alternatively, or in addition, the probiotic bacteria may be freeze-dried. The term “freeze-drying” (also known as lyophilisation, lyophilization, or cryodesiccation) is used in its regular meaning as the cooling of a liquid sample, resulting in the conversion of freeze-able solution into ice, crystallization of crystallisable solutes and the formation of an amorphous matrix comprising non-crystallizing solutes associated with unfrozen mixture, followed by evaporation (sublimation) of water from amorphous matrix. In this process the evaporation (sublimation) of the frozen water in the material is usually carried out under reducing the surrounding pressure to allow the frozen water in the material to sublimate directly from the solid phase to the gas phase. Freeze-drying typically includes the steps of pretreatment, freezing, primary drying and secondary drying. Methods of freeze-drying are known in the art. An exemplary method is described in WO2015000972, the full contents on which is incorporated by reference herein.

In other examples, the probiotic bacteria may be spray-dried or extruded.

### Compositions

Also disclosed herein are compositions (e.g., probiotic compositions) comprising the recombinant conjugative plasmids and probiotic bacteria of the present disclosure. Such compositions may be used to prevent or reduce the spread and/or acquisition of plasmids conferring undesirable traits (e.g., such as pathogenic plasmids as described herein) in a bacterial population. For example, the compositions may be used to prevent or reduce the spread and/or acquisition of plasmids that confer multi-drug resistance (also referred to as AMR), virulence and/or metal resistance in pathogenic bacterial populations and assists with the colonization (i.e. re-colonization) of the gastrointestinal tract or other environments (e.g., such as in the case of bioremediation).

In some examples, the compositions may be formulated for administration to a subject. For example, the probiotic bacteria of the disclosure may be formulated in a composition suitable for administration to a human or animal subject. In one example, the composition is for administration to a human. In another example, the composition is for administration to an animal. Exemplary animals for which the compositions of the disclosure may be particularly useful include livestock species (e.g. cattle, sheep, horses, pigs, donkeys, poultry), companion animals (e.g. dogs, cats), performance animals (e.g. racehorses, camels, greyhounds) and captive wild animals. In one example, the animal is a ruminant. Exemplary ruminants include cattle, sheep, goats, buffalo, deer or camelids. In another example, the animal may be a hind gut fermenter. An exemplary hindgut fermenter is a horse. In another example, the animal may be an avian species, such as poultry.

In other examples, the compositions may be formulated for administration to an environment selected from a healthcare environment, a soil environment, an environment comprising a water source, an environment comprising waste water, an environment comprising industrial waste, an environment comprising agricultural waste, an environment comprising sewerage and/or an environment comprising biosolids.

The probiotic bacteria and compositions comprising same may be formulated for administration or application by any route determined to be suitable by a person skilled in the art. For example, in accordance with examples in which the probiotic bacteria and compositions comprising same are for administration to a subject, the composition may be formulated for oral administration (e.g., as an ingestible liquid or solid, an oral drench, a feed additive, a food (e.g., a dairy product such as a drinkable yoghurt), or a capsule), topical administration (e.g., as a lotion or cream), intranasal administration or parenteral administration. In one example, the composition of the disclosure is formulated for oral administration e.g., as a food, beverage, bolus, drench or capsule. Accordingly, the composition may further comprise one or more physiologically acceptable excipients, carriers or additives suitable for ingestion by a human or non-human animal. Physiologically acceptable excipients, carriers or additives suitable for ingestion by human or non-human animals are known in the art and described herein. Such carriers can, for example, allow the probiotic bacteria of the disclosure to be formulated as tablets, pills, dragées, capsules, liquids, gels, syrups, slurries, suspensions and the like. The choice of carrier will be dependent on the form of the composition, the intended method of administration, the intended shelf-life and storage considerations. In some examples, the composition may be a food or beverage product (e.g., a dairy product such as a drinkable yoghurt). In some examples, the composition may be a tablet, pill, caplet, or capsule. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinylpyrrolidone, agar, or alginic acid or a salt thereof such as sodium alginate. Compositions that can be used orally include, but are not limited to, capsules made of gelatin, as well as soft, sealed capsules made of gelatin and a plasticizer, such as glycerol or sorbitol. In some examples, the composition may be formulated in a buffer. It will be understood by a person skilled in the art that by suitable buffer may be used. Examples of suitable buffers include, but are not limited to phosphate, calcium carbonate, bicarbonate, phosphate citrate and histidine. In other examples, the composition may be formulated with a carrier having a low oxygen diffusion rate e.g., such as ingestible oils. The composition may further comprise an antioxidant.

In some examples, the composition may comprise a preservative or a stabilizer. Furthermore, depending on the method of manufacture, the composition may comprise one or more cryoprotectants known in the art.

In accordance with an example in which the composition is a food product (such as a diary product), feed/nutritional supplement or beverage product, the probiotic bacteria or a composition comprising same may be prepared by, or shipped to, a manufacturer. The probiotic bacteria or composition comprising the same may then be formulated into the food product, feed/nutritional supplement or beverage product by the addition of further ingredients which are appropriate to the product.

In some examples, the composition of the disclosure is stable when stored at ambient room temperature (e.g., 20° C. and 25° C.) e.g., stable when stored at ambient room temperature for at least one month or more. In other examples, such as those in which the composition is formulated as a dairy food or beverage product, the composition may be required to be refrigerated in order to maintain viability of the probiotic bacteria.

In some examples, a composition of the disclosure is packaged in a container. The container may contain a single dose or multiple doses of the composition as described herein.

Preferably the compositions comprise probiotic bacteria of the disclosure in an amount sufficient to at least partially provide a benefit to the health of a subject or the environment to which they are administered.

In accordance with applications in which the probiotic composition is formulated for use in a human or non-human animal, the compositions will comprise probiotic bacteria in an amount sufficient to at least partially provide a health benefit to the human or non-human animal. An amount adequate to accomplish this is defined as a “therapeutically effective amount”. Effective amounts for this purpose may vary depending on a number of factors known to those skilled in the art, including, but not limited to, the species of subject, anatomy of the digestive system (e.g., four chamber or single chamber stomach), the size/weight of the subject, the composition of the subject's diet (existing and future), whether the subject is lactating, whether the subject is pregnant and the outcome to be achieved. The appropriate dosage of the probiotic bacteria (e.g., CFUs per strain) to be formulated in a composition of the disclosure may therefore be determined by a person skilled in the art, taking into account one or more of the above factors.

In one example, a unit or dosage of a composition of the disclosure may comprise between about 10² CFU to about 10¹⁴ CFU, or about 10³ CFU to about 10¹³ CFU, or about 10⁴ CFU to about 10¹³ CFU, or about 10⁵ CFU to about 10¹³ CFU, or about 10⁶ CFU to about 10¹³ CFU, or about 10⁶ CFU to about 10¹² CFU, or about 10⁷ CFU to about 10¹¹ CFU, or about 10⁸ CFU to about 10¹⁰ CFU, or about 10⁹ CFU to about 10¹⁰ CFU of the probiotic bacteria of the disclosure. For example, each unit or dosage of a composition of the disclosure may comprise about 5×10⁷ CFU or about 6×10⁸ CFU, or about 10⁹ CFU, or about 10¹⁰ CFU of the probiotic bacteria.

### Methods

Also provided herein are methods for preventing or reducing the spread and/or acquisition of plasmids conferring undesirable traits (e.g., such as pathogenic plasmids as described herein) in a bacterial population, said method comprising introducing to the population a probiotic bacteria or composition comprising same as described herein.

In one example, the method is for preventing or reducing the spread and/or acquisition of plasmids conferring antimicrobial resistance in a bacterial population. For example, the method may comprise introducing to the population of bacteria a probiotic bacteria or composition comprising same as described herein, wherein one or more of the probiotic bacterial cells introduced to the population comprises a recombinant conjugative plasmid comprising entry exclusion systems (EES) from a plurality of plasmid variants conferring AMR, as described herein.

In one example, the method is for preventing or reducing the spread and/or acquisition of plasmids conferring bacterial virulence in a bacterial population. For example, the method may comprise introducing to the population a probiotic bacteria or composition comprising same as described herein, wherein one or more of the probiotic bacterial cells introduced to the population comprises a recombinant conjugative plasmid comprising EES from a plurality of plasmid variants conferring bacterial virulence, as described herein.

In another example, the method is for preventing or reducing the spread and/or acquisition of plasmids conferring heavy metal resistance in a bacterial population. For example, the method may comprise introducing to the population of bacteria a probiotic bacteria or composition comprising same as described herein, wherein one or more of the probiotic bacterial cells introduced to the population comprises a recombinant conjugative plasmid comprising EES from a plurality of plasmid variants conferring heavy metal resistance, as described herein.

For example, the method may prevent or reduce the spread and/or acquisition of plasmids conferring pathogenic traits (such as AMR and/or bacterial virulence) in a bacterial population of the gastrointestinal tract during conjugation rounds. In one example, the method may prevent or reduce the spread and/or acquisition of plasmids conferring pathogenic traits to bacteria of the family of Enterobacteriaceae. The family “Enterobacteriaceae” includes 14 main genera and 6 further genera, and is known to have different properties. Typical examples are Escherichia, Salmonella and Klebsiella. Virulence genes are usually found in enterobacteria on large plasmids (approx. 60 kb or larger). Enterobacteria are generally pathogens which infect the gastrointestinal tract of avians and/or mammals. The method of the present disclosure can be utilised to target populations of enterobacteria in the gut and introduce the recombinant conjugative plasmid to the progeny of the bacterial population in subsequent conjugation rounds. In doing so, the method may prevent or reduce the spread and/or acquisition of plasmids conferring pathogenic traits (such as AMR and/or bacterial virulence) the bacterial population of the gut.

In other examples, the method is for preventing or reducing the spread and/or acquisition of pathogenic plasmids as described herein in a bacterial population within the environment. The environment into which the probiotic bacteria or composition comprising same is introduced may be selected from a healthcare environment, a soil environment, an environment comprising a water source, an environment comprising waste water, an environment comprising industrial waste, an environment comprising agricultural waste, an environment comprising sewerage and/or an environment comprising biosolids. Accordingly, in one example, the method may be used for bioremediation.

A reduction in spread or acquisition of a pathogenic plasmid in a bacterial population may be any reduction, such as a reduction by at least 10% (such as by at least 15%, or at least 20%, or at least 25%, or at least 30%, or at least 35%, or at least 40% or more), relative to the prevalence of the pathogenic plasmid in bacterial population into which the probiotic bacteria or the disclosure has not been introduced. A reduction in spread or acquisition of a pathogenic plasmid in a bacterial population may result in one or more treatment outcomes. As used herein, the terms “treating”, “treat” or “treatment” and variations thereof, refer to clinical intervention using non-pathogenic bacteria designed to alter the natural course of the individual or cell being treated during the course of clinical pathology and can also refer to environmental remediation efforts. Desirable effects of treatment when used in connection with the biological organism or infection, includes the amelioration, elimination, reduction, prevention, or other relief or management from the detrimental effects of a biological organism via curing the pathogenic population off multi-drug resistance or metal resistance or virulence (for e.g., Enterobacteriaceae) either in the gut of a subject or other environments by ameliorating and/or controlling the colonization of pathogenic bacteria carrying such traits within the gastrointestinal tract of both humans or animals or other environments.

In one example, the probiotic bacteria or composition comprising same of the present disclosure is administered once or more daily, weekly, fortnightly, monthly, or bi-monthly, wherein a daily, weekly, fortnightly, monthly, or bi-monthly dosage comprises an amount of the probiotic bacteria as described above.

In some examples, the probiotic bacteria or composition comprising the same is administered to a human or animal subject. In one example, the probiotic bacteria or composition is administered to a human. In another example, the probiotic bacteria or composition is administered to an animal. Exemplary animals for which the probiotic bacteria and compositions of the disclosure may be particularly useful are described herein.

As described herein, the probiotic bacteria and compositions may be administered by any route determined to be suitable by a person skilled in the art. For example, in accordance with examples in which the probiotic bacteria and compositions are administered to a human or animal subject, the composition may be formulated for administration orally (e.g., as an ingestible liquid or solid, an oral drench, a feed additive, a food (e.g., a dairy product), or a capsule), topically (e.g., as a lotion, cream or gel), intranasally or parenterally. In one example, the composition of the disclosure is administered orally e.g., as a food, beverage, bolus, drench or capsule.

In one example, the probiotic bacteria or composition comprising same may be administered as topical cream, lotion, or gel. For example, creams, lotions or gels comprising the probiotic bacteria may be effective in reducing or preventing the acquisition or spread of pathogenic plasmids within bacterial populations on the skin, thereby controlling antibiotic resistant pathogens on the skin. In this regard, pathogenic Pseudomonas, Staphylococcus, and/or Enterococci are frequently associated with infections of severe burns.

Preferably the probiotic bacteria is administered to the subject in an therapeutically effective amount. As described herein, this may vary depending on a number of factors known to those skilled in the art, including, but not limited to, the species of subject, anatomy of the digestive system (e.g., four chamber or single chamber stomach), the size/weight of the subject, the composition of the subject's diet (existing and future), whether the subject is lactating, whether the subject is pregnant and the outcome to be achieved. The appropriate dosage of the probiotic bacteria (e.g., CFUs per strain) to be formulated in a composition of the disclosure may therefore be determined by a person skilled in the art taking into account one or more of the above factors.

In one example, a unit or dosage of a composition administered to a subject may comprise between about 10² CFU to about 10¹⁴ CFU, or about 10³ CFU to about 10¹³ CFU, or about 10⁴ CFU to about 10¹³ CFU, or about 10⁵ CFU to about 10¹³ CFU, or about 10⁶ CFU to about 10¹³ CFU, or about 10⁶ CFU to about 10¹² CFU, or about 10⁷ CFU to about 10¹¹ CFU, or about 10⁸ CFU to about 10¹⁰ CFU, or about 10⁹ CFU to about 10¹⁰ CFU of the probiotic bacteria of the disclosure. For example, the method may comprise administering a composition comprising about 5×10⁷ CFU or about 6×10⁸ CFU, or about 10⁹ CFU, or about 10¹⁰ CFU of the probiotic bacteria.

### EXAMPLES

### Example 1: Probiotic Plasmid with Single Exclusion Gene Inhibits IncF Plasmid Transfer

Probiotic plasmids pJIMK_traS_F, pJIMK_traS_R100, pJIMK_traS_SLT were constructed by cloning different traS variants from IncF plasmids into a pBCSK+ backbone.

### Bacterial Strains and Plasmids Used:

Table 1 provides details of the various bacterial strains and plasmids used to generate probiotic plasmids designated pJIMK_traS_F, pJIMK_traS_R100, pJIMK_traS_SLT.

TABLE 1
Bacterial strains and plasmids used

| Bacterial strains/ | |
|---|---|
| Plasmids | Characteristics |
| J53 | Sodium azide-resistant E. coli-K12 strain |
| BW25113Rf | Rifampicin-resistant E. coli-K12 strain |
| pBCSK+ | Chloramphenicol-resistant cloning vector (Stratagene, USA) |
| pBAD33_Gm | Gentamicin-resistant expression vector with p15A origin of |
| | replication and L-arabinose inducible promoter upstream of the |
| | multiple-cloning site (Addgene Plasmid #65098) |
| R100 | IncF plasmid from Shigella flexneri (GenBank accession no. |
| | AP000342.1) |
| F-like plasmid | IncF plasmid from E. coli strain with the identical traS of E. coli- |
| | K12 plasmid F (GenBank accession no. AP001918.1) |
| pSLT-like | IncF plasmid from Salmonella typhimurium isolate carrying the |
| | identical traS of pSLT plasmid (GenBank accession |
| | no. AE006471.2) |

### Analysis of Entry Exclusion Systems in IncF Plasmids:

The inventors obtained and examined traS sequences, which are responsible for entry exclusion in IncF plasmids, for those IncF plasmids which are available in GenBank and then performed multiple sequence alignments using MEGAX software. BlastN and BlastP searches were then performed on the traS nucleotides and amino acid sequences. Three major variants of traS sequences were identified from the IncF plasmids sequences available in GenBank. These three traS variants were named traS_F, traS_R100 and traS_SLT. Variant traS_F was obtained from E. coli K-12 plasmid F (GenBank accession no. AP001918.1), traS_R100 was obtained from Shigella flexneri plasmid R100 (GenBank accession no. AP000342.1), and traS_SLT was obtained from Salmonella typhimurium plasmid pSLT (GenBank accession no. AE006471.2). The complete nucleotide sequences of these three trsS variants designated traS_R100, traS_F, and traS_SLT are set forth in SEQ ID NOs: 1-3, respectively.

### Construction of Probiotic Plasmids:

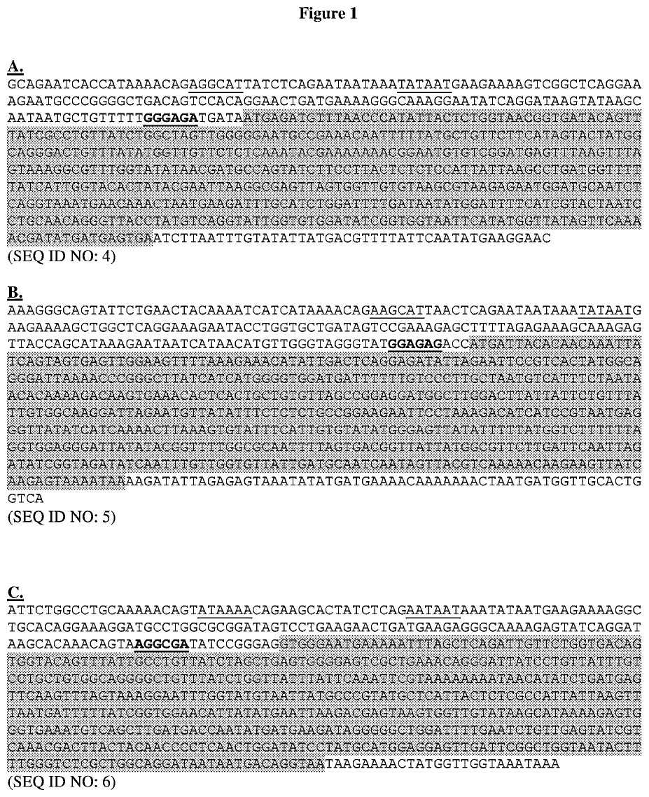

gBlocks elements (IDT, USA) of traS_R100, tras_F and traS_SLT genes with their native promoter and ribosome binding sites (RBS) were artificially synthesised with appropriate restriction sites (FIG. 1A-C, and SEQ ID NOs: 4-6 respectively).

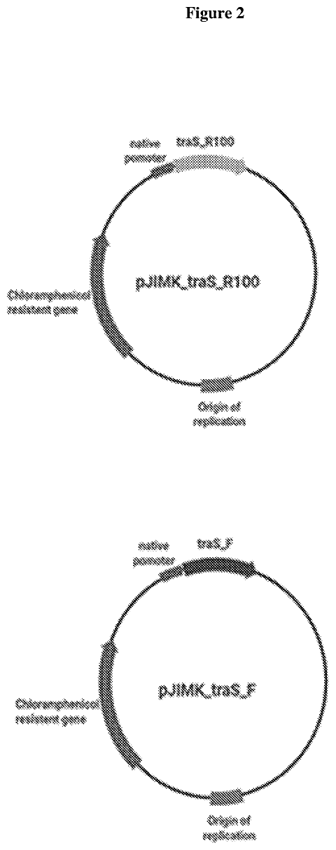

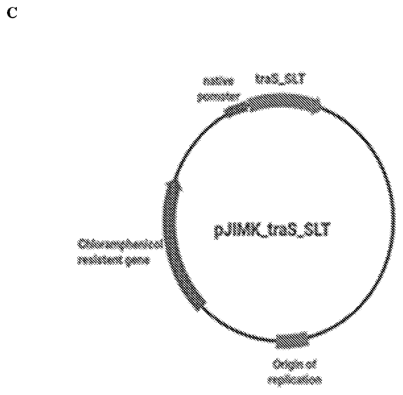

The synthesised traS_R100, traS_SLT and traS_F genes were then digested with HindIII+BamHI, BamHI+EcoRI, and BamHI+XbaI restriction enzymes combinations, respectively, at 37° C. for 1 hr. Digested DNA samples were then purified using PCR purification kit (Thermo Fisher, USA). At the same time, the cloning vector pBCSK+(Chloramphenicol-resistant) was digested with respective restriction enzymes combinations for 2 hr and then purified. Purified traS gene variants were then cloned separately into the cloning vector pBCSK+ to construct the probiotic plasmids pJIMK_traS_R100, pJIMK_traS_F and pJIMK_traS_SLT (FIG. 2). These probiotic plasmids were transformed into rifampicin-resistant E. coli BW25113Rf to produce BW25113Rf (pJIMK_traS_R100), BW25113Rf (pJIMK_traS_F), BW25113Rf (pJIMK_traS_SLT). The BW25113Rf with probiotic plasmids and an empty BW25113Rf were then used as the recipient for the transfer of respective IncF plasmids from the donor J53 strains (Table 2).

TABLE 2
IncF plasmid specific entry exclusion system in the recipient
bacteria prevent the acquisition of respective IncF plasmid

| | | Conjugation | Exclusion index/ |
|---|---|---|---|
| Donor | Recipient | frequency | protection index |
| J53(R100) | BW25113Rf | (4.6 ± 1.7) × 10⁻² | 1 |
| J53(R100) | BW25113Rf(pJIMK_traS_R100) | (2.2 ± 1.1) × 10⁻⁴ | 209 |
| J53(F-like plasmid) | BW25113Rf | (5.2 ± 2.1) × 10⁻¹ | 1 |
| J53(F-like plasmid) | BW25113Rf(pJIMK_traS_F) | (2.4 ± 1.6) × 10⁻³ | 217 |
| J53(pSLT-like | BW25113Rf | (3.1 ± 0.7) × 10⁻³ | 1 |
| plasmid) | | | |
| J53(pSLT-like | BW25113Rf(pJIMK_traS_SLT) | (1.2 ± 0.5) × 10⁻⁵ | 258 |
| plasmid) | | | |

### In Vitro Conjugation:

In vitro plasmid conjugation was measured using standard filter mating experiments (Rodriguez-Grande and Fernandez-Lopez 2020). The donor with respective plasmid and recipient bacteria (Table 2) were grown overnight in LB medium with appropriate antibiotics. Cultures (1 mL) were harvested by centrifugation at 10,000 g for 5 min, washed twice with 1 mL saline and suspended in 50 μL saline. Donor and recipient suspensions were mixed. The mixture was placed on a nitrocellulose filter (Amersham Hybond-C extra, 82 mm) on an LB agar plate and then incubated at 37° C. overnight. The mixture of cultures was harvested in 5 mL saline, and 100 μL of mating mixture with different dilutions were spread onto nutrient agar plates containing rifampicin (90 μg/mL) and plasmid specific antibiotic, which confers resistance to incoming plasmid and incubated at 37° C. overnight. Transconjugant colonies were then counted, and conjugation frequency was measured by dividing the number of transconjugants by the number of donor bacteria. Further, colonies were patched onto CHROMagar Orientation plates containing appropriate antibiotics to confirm that they were not spontaneous rifampicin-resistant mutants of donor strains. The presence of incoming plasmid genes in transconjugants was confirmed by colony PCR using single colonies as templates.

### Exclusion Index/Protection Index Measurement:

Exclusion index or protection index is the ratio of conjugation frequency for the empty recipient (without exclusion system) relative to the recipient bacteria with a probiotic plasmid (with cloned exclusion gene/s). The exclusion index indicates the fold difference in plasmid transfer inhibition. Thus, the higher exclusion index value indicates stronger protection from the acquisition of plasmid tested.

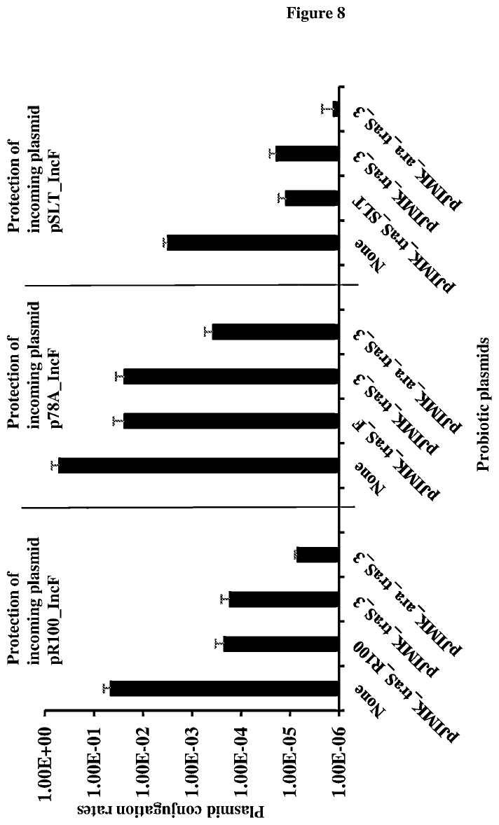

Probiotic plasmids with single exclusion gene inhibited the conjugation transfer of different IncF plasmid nearly 200 folds in the recipient bacteria (Table 2, FIG. 8). This transfer inhibition was specific to different plasmid types. For example, tras_R100 in recipient bacteria strongly inhibit the transfer of R100 plasmid.

### Example 2: Probiotic Plasmid with a Combination of Multiple Exclusion Gene Variants Inhibit Several IncF Plasmids from Conjugation Transfer

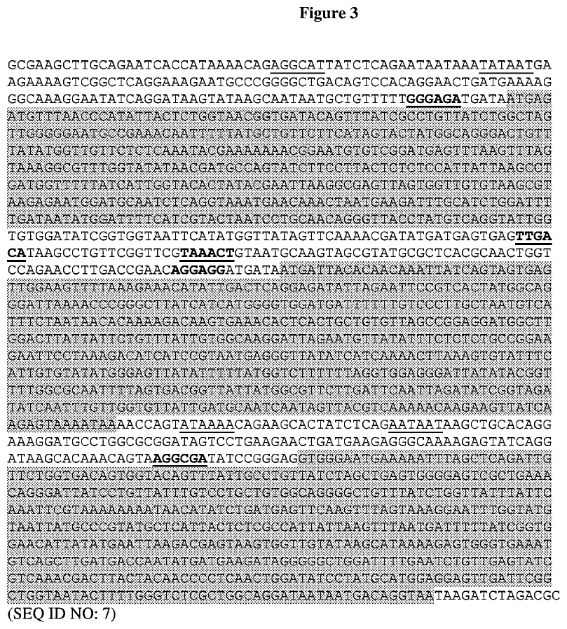

As the three variants of traS identified by the inventors (i.e., traS_R100, traS_SLT and tras_F) represented most of the IncF plasmids present in the GenBank database, the inventors combined all three traS gene variants of the IncF plasmids with their native promoters for traS_R100, traS_SLT and integron promoter Pc for traS_F with their native RBS into a single vector system and constructed a probiotic plasmid to protect host bacteria from acquisition of most IncF plasmids. The new probiotic plasmid, designated pJIMK_traS_3, was constructed by combining the three traS variants with their native promoters in two and one with integron promoter Pc and their own RBS with flanking HindIII and XbaI restriction sites into a single construct (SEQ ID NO: 7). Restriction digested and purified trsS-3 genes were then cloned into the HindIII+XbaI sites of pBCSK+ to construct pJIMK_traS_3 (FIGS. 3 and 4). The probiotic plasmid pJIMK_traS_3 was then transferred into BW25113Rf to construct BW25113Rf(pJTMK_traS_3) and used in the conjugation transfer of different TncF plasmid variants from J53 strain to measure the level of transfer inhibition. The inventors were able to show that bacteria carrying the probiotic plasmid pJIMK_traS_3 could successfully protect the recipient bacteria from acquiring all IncF plasmid variants tested similar to protection rendered by traS gene against specific F plasmids (Table 3, FIG. 8). This result suggests that probiotic plasmids with combinations of the different exclusion gene variants can be constructed to prevent the acquisition of various plasmid types. It also suggests that probiotic plasmids comprising the combination of three traS exclusion gene variants can prevent the acquisition of the majority of IncF plasmid types.

TABLE 3
Probiotic plasmid with multiple entry exclusion systems from IncF plasmid variants can
protect bacteria from the acquisition of several different IncF plasmid variants

| | | Conjugation | Exclusion index/ |
|---|---|---|---|
| Donor | Recipient | frequency | protection index |
| J53(R100) | BW25113Rf | (4.1 ± 1.4) × 10⁻² | 1 |
| J53(R100) | BW25113Rf(pJIMK_traS_3) | (1.7 ± 0.8) × 10⁻⁴ | 241 |
| J53(F-like plasmid) | BW25113Rf | (4.5 ± 1.1) × 10⁻¹ | 1 |
| J53(F-like plasmid) | BW25113Rf(pJIMK_traS_3) | (2.4 ± 1.2) × 10⁻³ | 187 |
| J53(pSLT-like | BW25113Rf | (4.3 ± 0.8) × 10⁻³ | 1 |
| plasmid) | | | |
| J53(pSLT-like | BW25113Rf(pJIMK_traS_3) | (1.9 ± 0.7) × 10⁻⁵ | 226 |
| plasmid) | | | |

### Example 3: Increased Expression of One or More Exclusion Genes in a Probiotic Plasmid Significantly Increase the Protection Against Target Plasmids

In this example, the inventors assessed whether an increase in expression of the exclusion traS variants increased the transfer inhibition of plasmids in recipient bacteria. Expression of a gene of interest can be conveniently controlled by cloning the gene into an expression vector under the arabinose inducible promoters with araB and araC genes. In the present case, the pBAD33_Gm expression vector (Gentamicin-resistant, Plasmid #65098, Addgene) was used to produce expression vectors comprising exclusion gene traS variants to control the expression of the traS variant genes with the addition of different concentrations of arabinose.

The effect of traS gene expression on transfer inhibition of plasmids in recipient bacteria was specifically assessed for the variant traS_R100. Briefly, the traS_R100 sequence with consensus RBS region was synthesised and cloned into the HindIII+XbaI sites of pBAD33_Gm to construct pJIMK_ara_traS_R100 (FIG. 5). It was found that increased expression of traS_R100 in the recipient bacteria from the probiotic plasmid pJIMK_ara_traS_R100 with arabinose (0.2%) significantly increased the inhibition of R100 plasmid transfer to the recipient bacteria (Table 4). This result suggests that high-levels of traS exclusion protein in the recipient bacteria can significantly improve the level of protection from the acquisition of target plasmid (R100).

TABLE 4
High-level expression of exclusion gene in the recipient bacteria can significantly
increase the protection from the acquisition of respective plasmid (R100)

| | | Conjugation | Exclusion index/ |
|---|---|---|---|
| Donor | Recipient | frequency | protection index |
| J53(R100_IncF) | BW25113Rf | (4.6 ± 1.7) × 10⁻² | 1 |
| J53(R100_IncF) | BW25113Rf(pJIMK_traS_R100) | (2.2 ± 1.1) × 10⁻⁴ | 209 |
| J53(R100_IncF) | BW25113Rf(pJIMK_ara_traS_R100) | (2.1 ± 0.5) × 10⁻⁵ | 2190 |

To improve the expression of all three traS variants from a single probiotic plasmid to deliver high-level protection against the acquisition of most of the IncF plasmids, the three traS variants were synthesised with araBAD promoters (FIG. 6) and cloned into pBAD33_Gm to construct pJIMK_ara_traS_3 (FIG. 7). More specifically, the triple traS construct comprised three traS variants (i) a consensus RBS with traS_R100 sequence, (ii) traS_F with its native promoter and RBS, and (iii) traS_SLT with araBAD promoter and consensus RBS, with HindIII and XbaI restriction sites flanking the three traS variant sequences (SEQ ID NO: 8). The triple traS construct was then restriction digested, purified and cloned into the HindIII+XbaI sites of pBAB33_Gm to construct pJIMK_ara_traS_3 (FIG. 7).

In this probiotic plasmid, traS_R100 and traS_SLT were regulated directly from araBAD promoter, whereas traS_F was under the control of the native promoter and araBAD promoter. The probiotic plasmid pJIMK_ara_traS_3 was then transferred into the BW25113Rf strain, which was used as a recipient in the conjugation transfer of different IncF plasmids from E. coli J53 (Table 5). The recipient bacteria were induced with 0.2% arabinose for one hour before the mixing donor and recipients for mating experiments. Surprisingly, it was found that induction of traS_3 genes with arabinose considerably increased the conjugation transfer inhibition of all three different IncF plasmids tested (Table 5, FIG. 8). This suggests that increasing the expression of the exclusion gene/s from a probiotic plasmid in the bacteria can improve the protection from the acquisition of any target plasmids.

TABLE 5
High-level expression of multiple entry exclusion systems of IncF plasmid
variants from a probiotic plasmid can significantly increase the protection
of bacteria from acquisition of several different IncF plasmid variants

| | | Conjugation | Exclusion index/ |
|---|---|---|---|
| Donor | Recipient | frequency | protection index |
| J53(R100) | BW25113Rf | (4.4 ± 1.5) × 10⁻² | 1 |
| J53(R100) | BW25113Rf(pJIMK_ara_traS_3) | (7.1 ± 0.8) × 10⁻⁶ | 6197 |
| J53(F-like plasmid) | BW25113Rf | (4.5 ± 1.1) × 10⁻¹ | 1 |
| J53(F-like plasmid) | BW25113Rf(pJIMK_ara_traS_3) | (3.7 ± 1.8) × 10⁻⁴ | 1216 |
| J53(pSLT-like | BW25113Rf | (4.6 ± 1.2) × 10⁻³ | 1 |
| plasmid) | | | |
| J53(pSLT-like | BW25113Rf(pJIMK_ara_traS_3) | (1.3 ± 0.9) × 10⁻⁶ | 3638 |
| plasmid) | | | |

### Example 4: Conjugative Probiotic Plasmid to Protect Bacteria from the Invasion of IncM, IncL, IncC and IncA Plasmid Types In Vitro

In this example, the inventors designed and constructed conjugative probiotic plasmids comprising Exc protein sequences from multiple Inc plasmid types and evaluated the ability of those probiotic plasmids to prevent bacteria from being invaded by AMR plasmids of multiple incompatibility types.

### Analysed the Exc Protein Sequences for the IncM, L, C and A Plasmid Types

The inventors analysed the Exc protein sequences for IncM, IncL, IncC and IncA plasmid types which were available in Genbank.

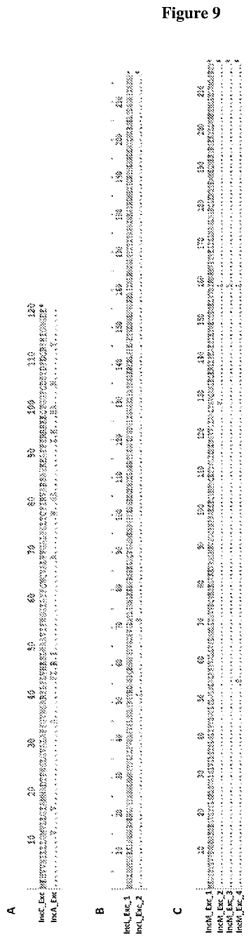

Based on this analysis, the inventors identified that all IncC plasmids in GenBank carried a single Exc protein type. The Exc of IncA is slightly different from the Exc of IncC plasmids (See FIG. 9A), but a recent experimental study identified that despite some amino acid sequence variations, both of the Exc proteins fall into the same Exc group and demonstrate that IncC Exc can efficiently exclude both IncC and IncA group plasmids (Humbert et al., 2019).

IncL plasmids were also predominant represented by a single Exc type (See IncL_Exc_1 in FIG. 9B). In this regard, 95% (233/249) of the IncL plasmids in GenBank carried this unique type of Exc protein, whereas 5% (16/249) of the Inc Exc possessed 1-2 aa variations relative to the dominant Exc type. This suggests that all Exc variants in IncL plasmids are closely related to each other and the major variant (IncL_Exc_1) may exclude all IncL plasmids.

The IncM plasmids carried two major types of Exc proteins (See FIG. 9C) which varied by two amino acid only and which collectively represented >99% of the IncM plasmids in GenBank.

The inventors predicted the promoter region associated with these major Exc variants in the IncL, M, and C plasmids.

Construction of a Conjugative Probiotic Plasmid to Prevent Bacteria from Invasion of IncM, L, C and A Plasmid Types

The inventors previously identified an in vivo conjugation efficient IncM plasmid pJIBE401. In this study, the conjugation efficient IncM plasmid backbone of pJIBE401 was used as a backbone to construct a probiotic plasmid to protect bacteria from the invasion of the four different plasmid types. A ˜28.0 kb multi-resistance region (MRR) carrying multiple antibiotic resistance genes and transposable elements was deleted from the IncM plasmid pJIBE401, and replaced with a tetracycline resistance gene (tetA), to produce the plasmid backbone designated pJIMK45 (Kamruzzaman et al., 2017).

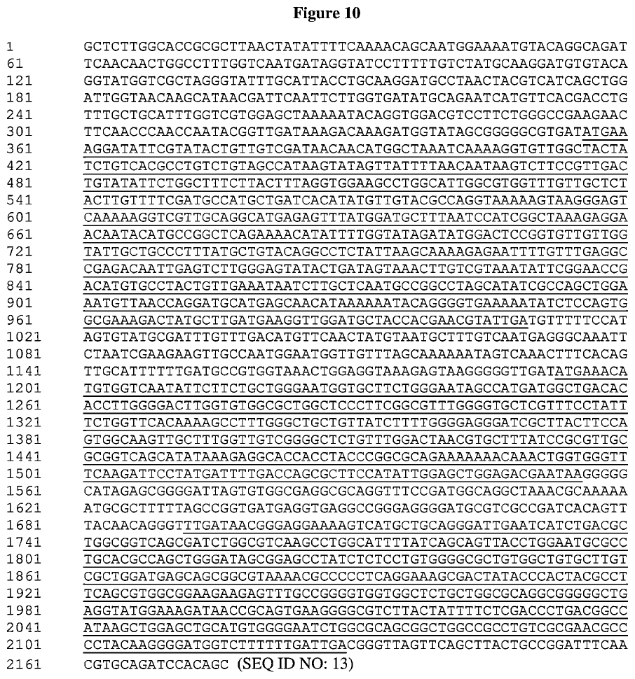

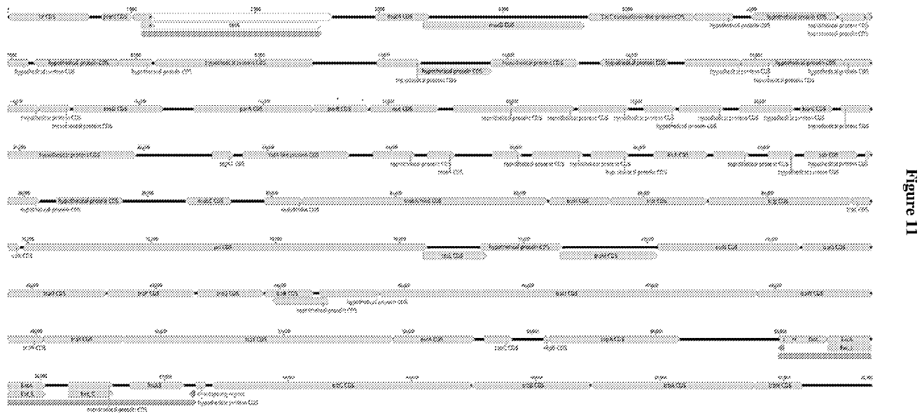

The inventors used pJIMK45 as the backbone for their conjugative probiotic plasmid. This backbone already contained the exc gene for IncM plasmid type. The inventors then introduced the exc genes from IncL and IncC plasmids to produce the conjugative probiotic plasmid (PB1). Briefly, the exc gene sequences from the IncL and IncC plasmids, together with their native promoter and ribosome binding sites, were combined with afosA3 gene, and flanked on both sides by 60 bp sequences homologous to regions flanking the tetA gene in pJIMK45 (the synthetic sequence was designated “gBlock”; FIG. 10). The gBlock was synthesized commercially and introduced to the pJIMK45 backbone plasmid using homologous recombination-based allelic exchange method, where it replaced the tetA gene. To insert the gBlock, the E. coli UB5201Rf strain carrying pJIMK45 was transformed with a lambda red recombinase plasmid pKM200 (Chloramphenicol resistant) and selected on tetracycline plus chloramphenicol plates at 30° C. Electro competent cells were prepared from UB5201Rf(pJIMK45+pKM200) and transformed with the PCR amplified gBlock of excL-excC-fosA3 by electroporation. The transformant was selected on Fosfomycin-resistant plates. The selected transformants were then confirmed for the sensitivity to tetracycline and confirmed the insertion event by PCR and sanger sequencing.

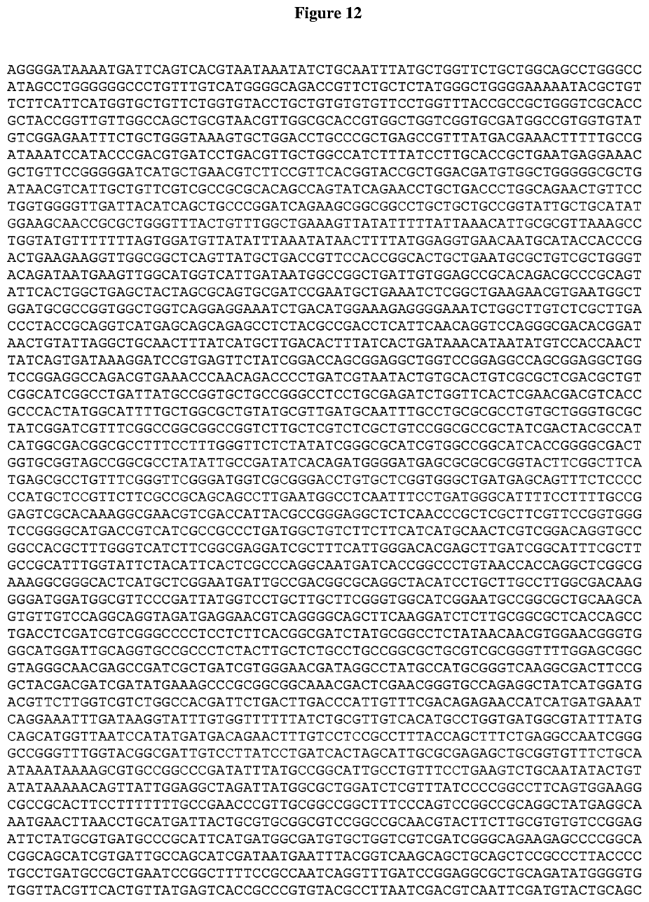

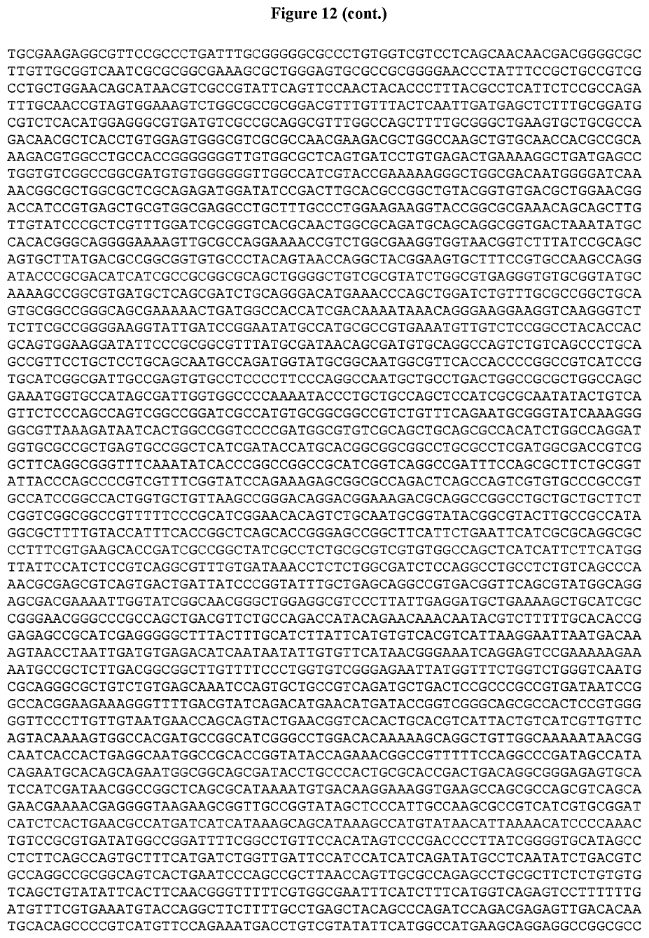

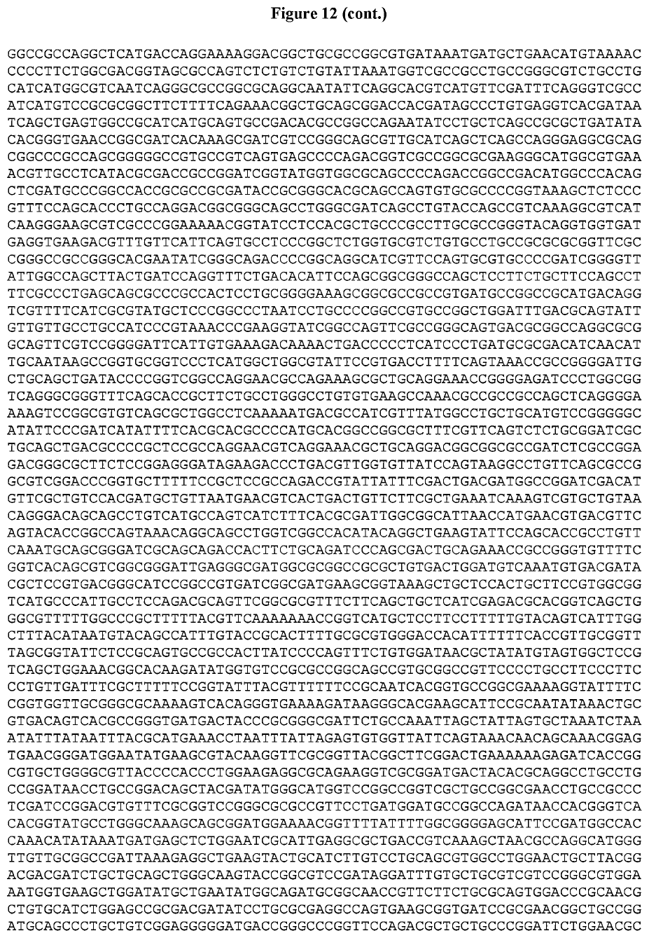

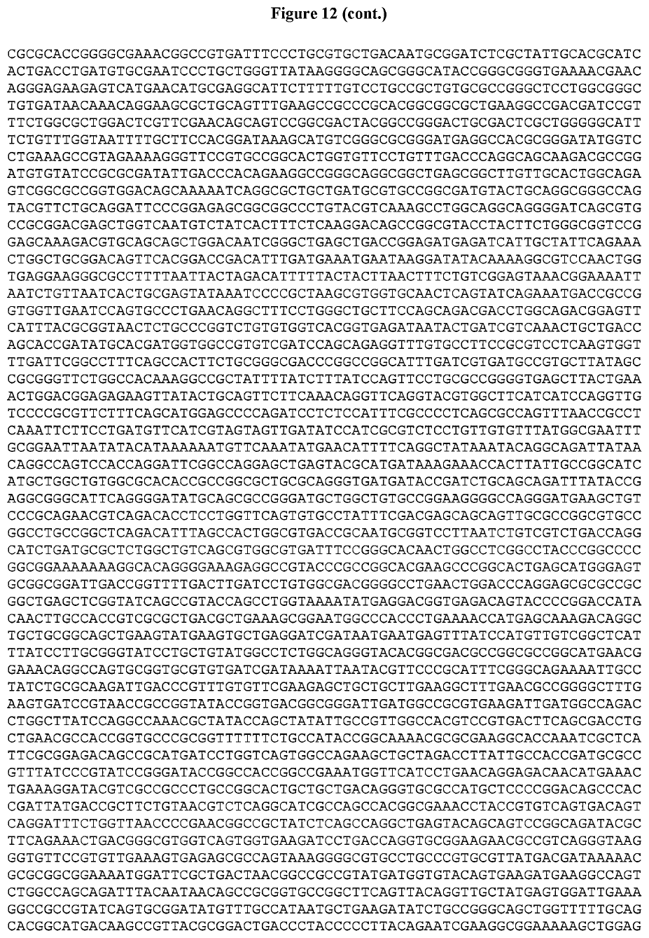

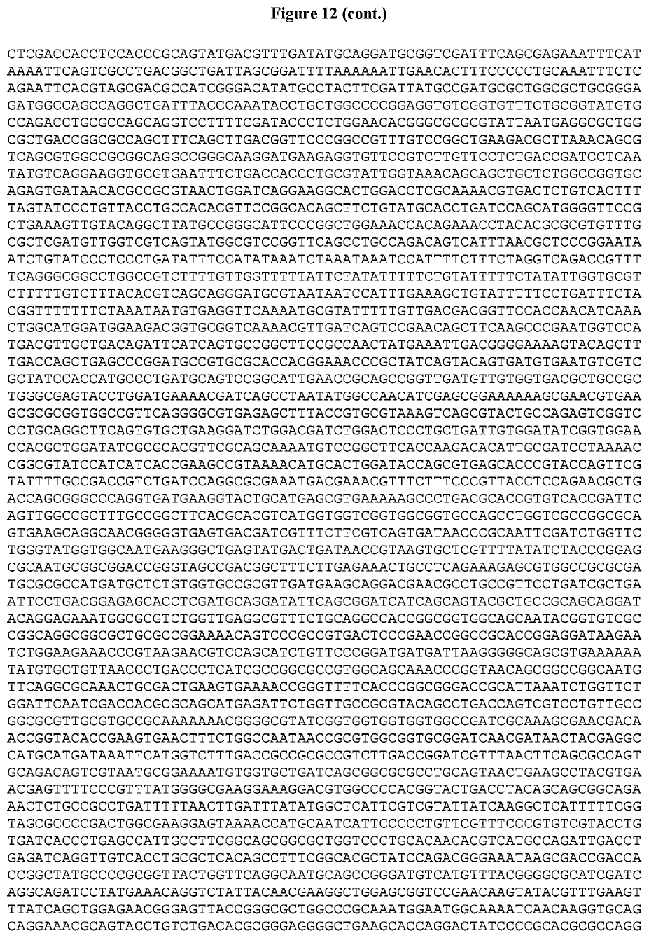

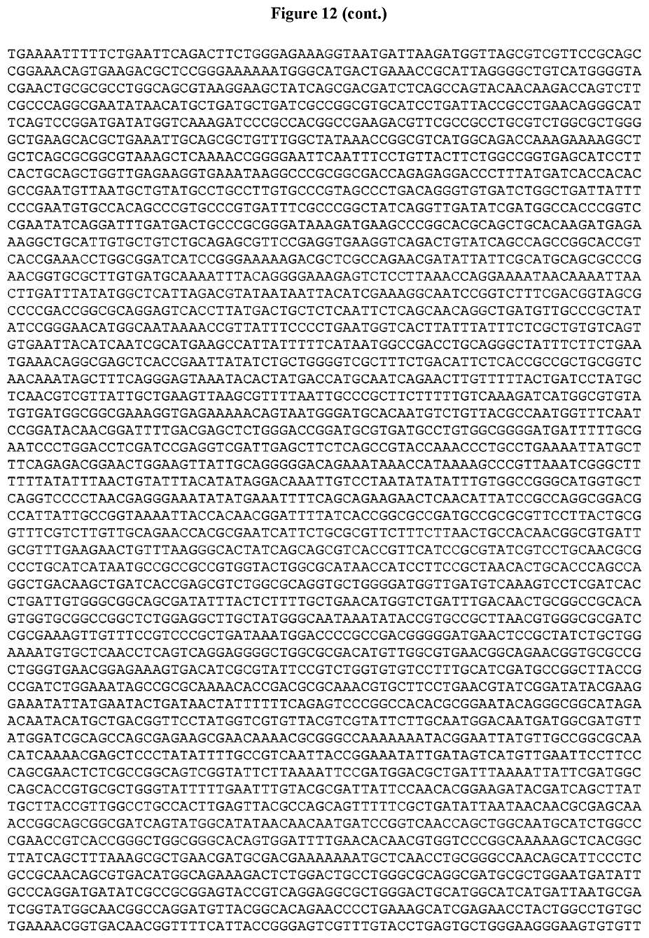

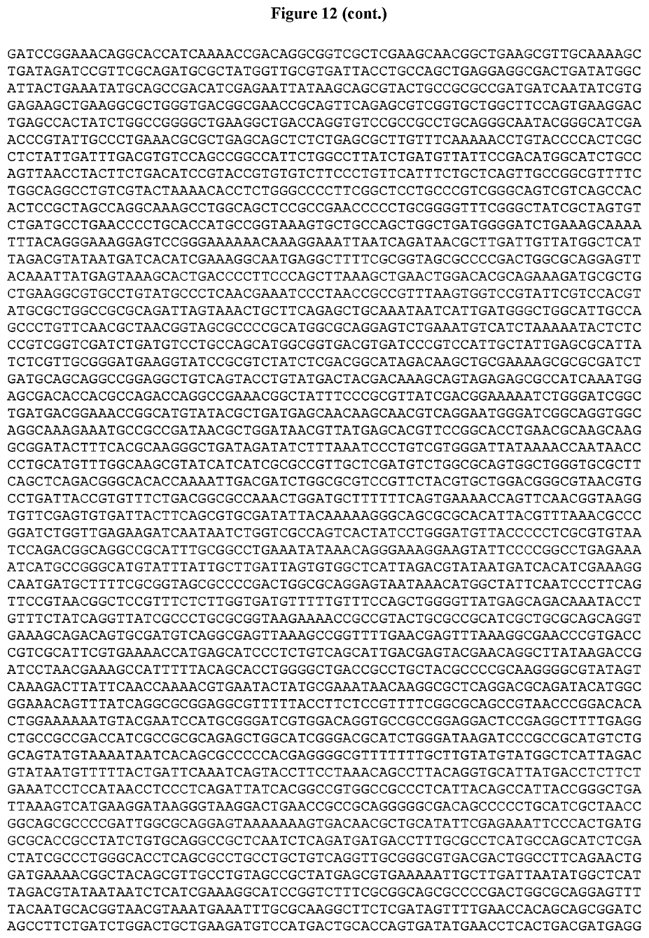

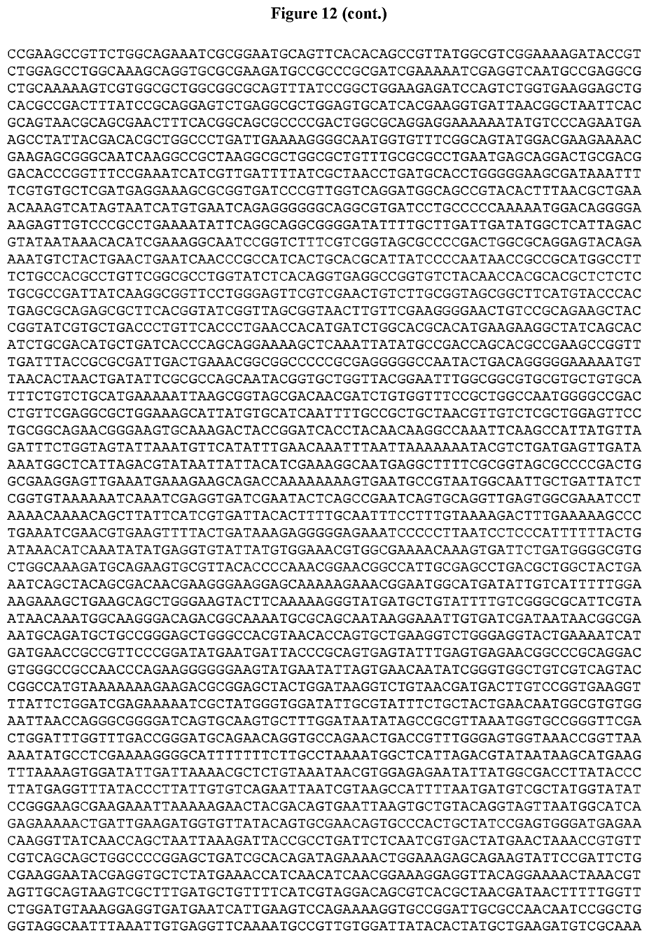

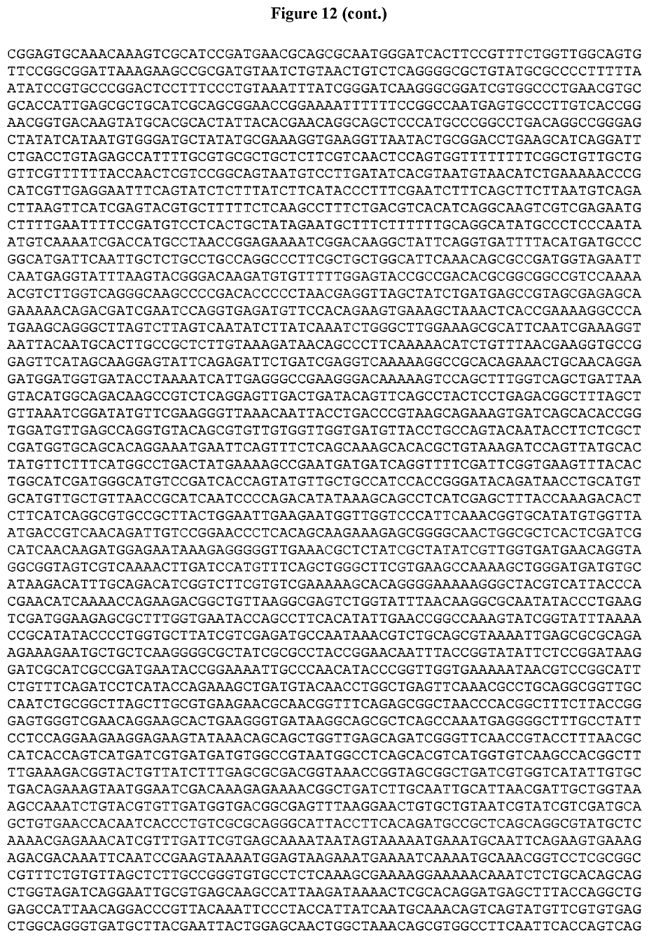

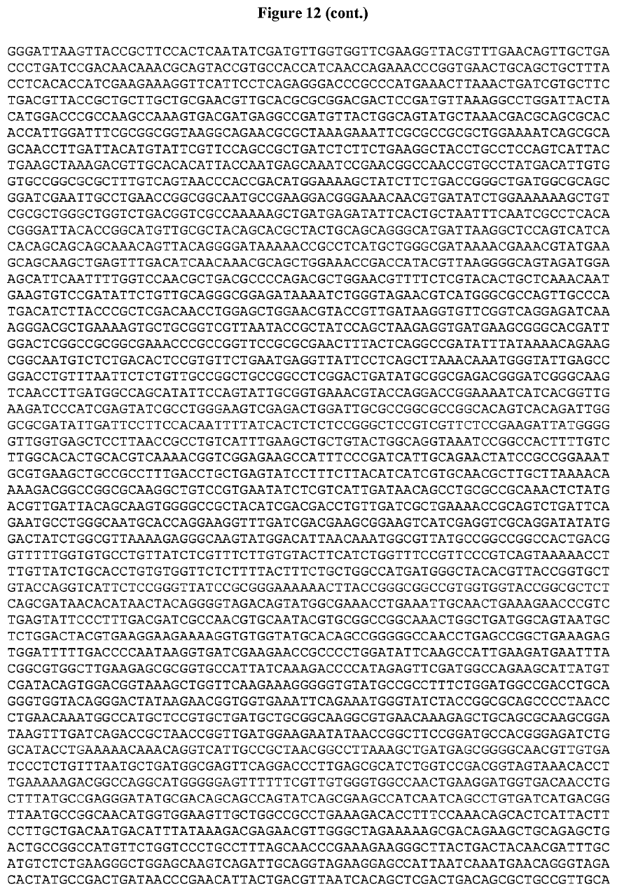

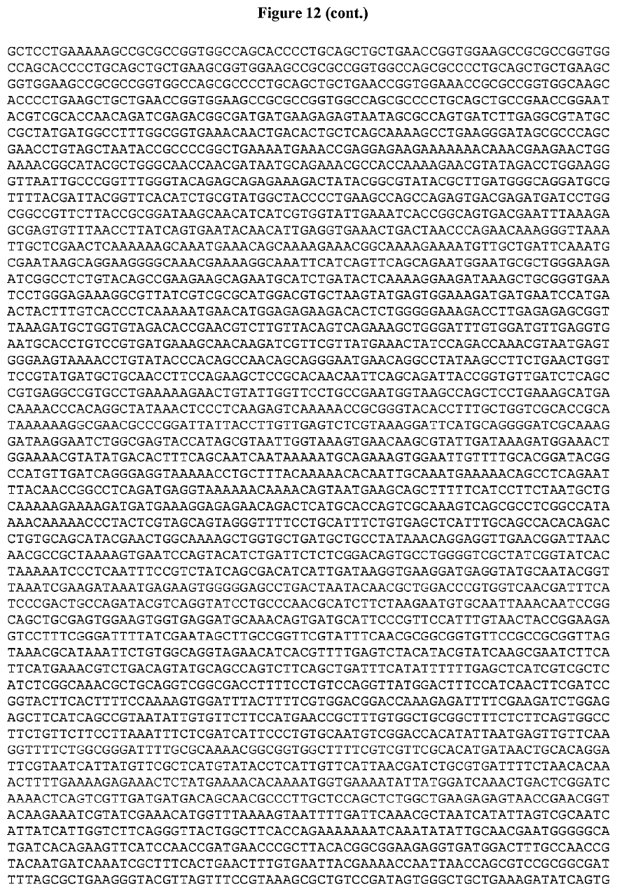

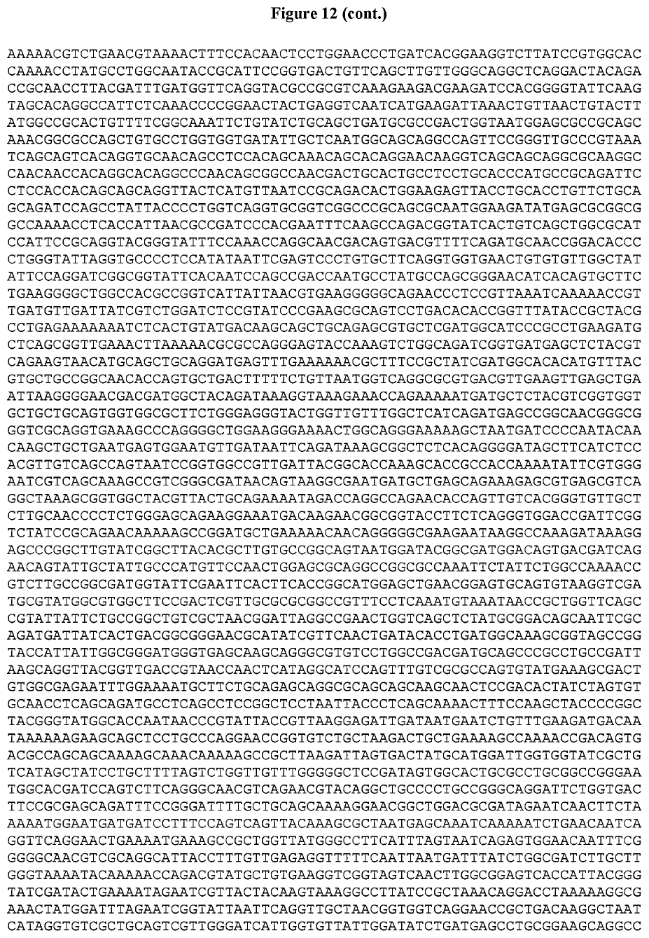

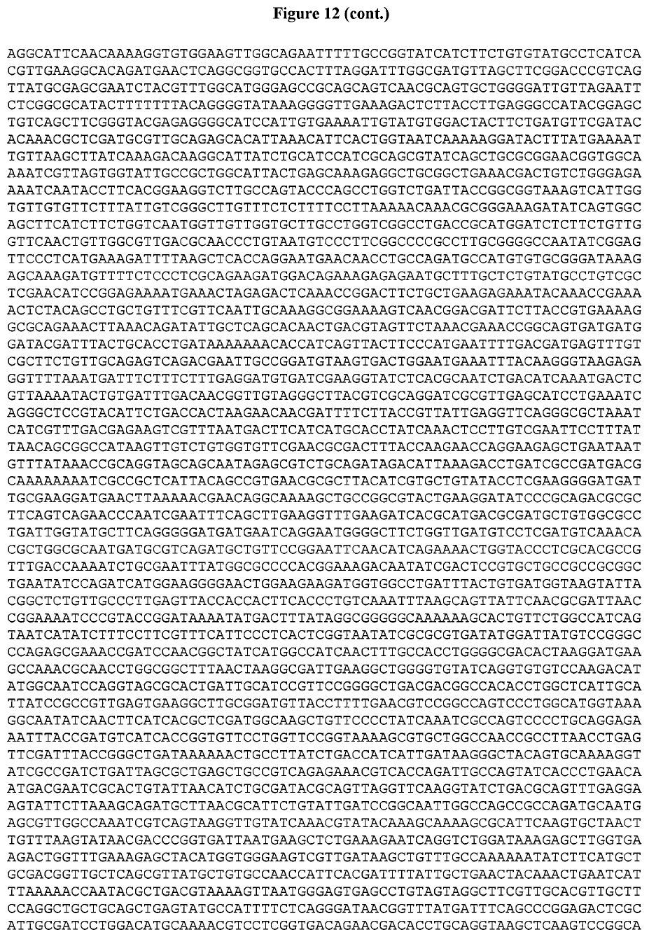

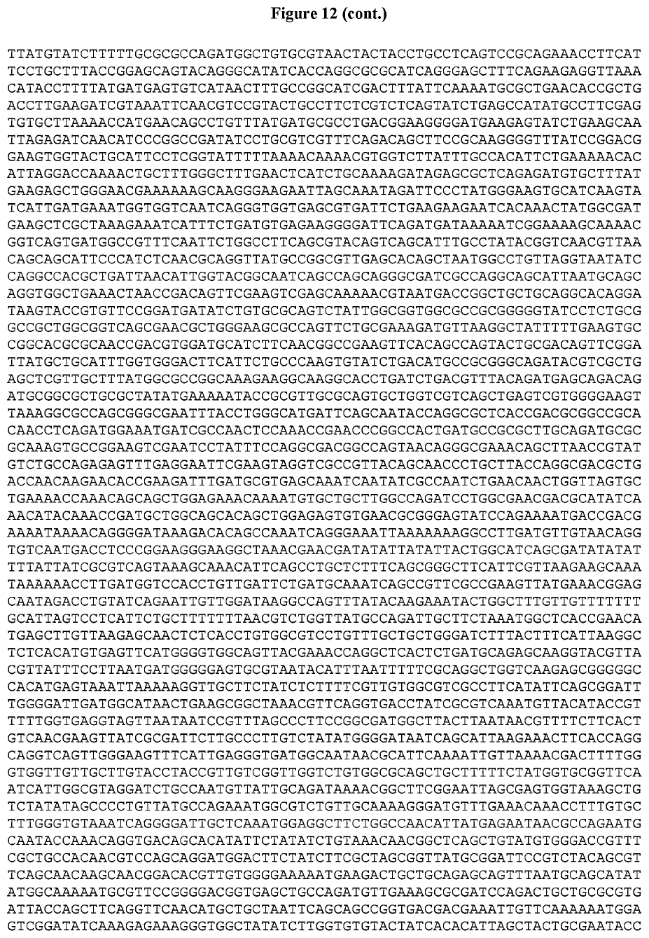

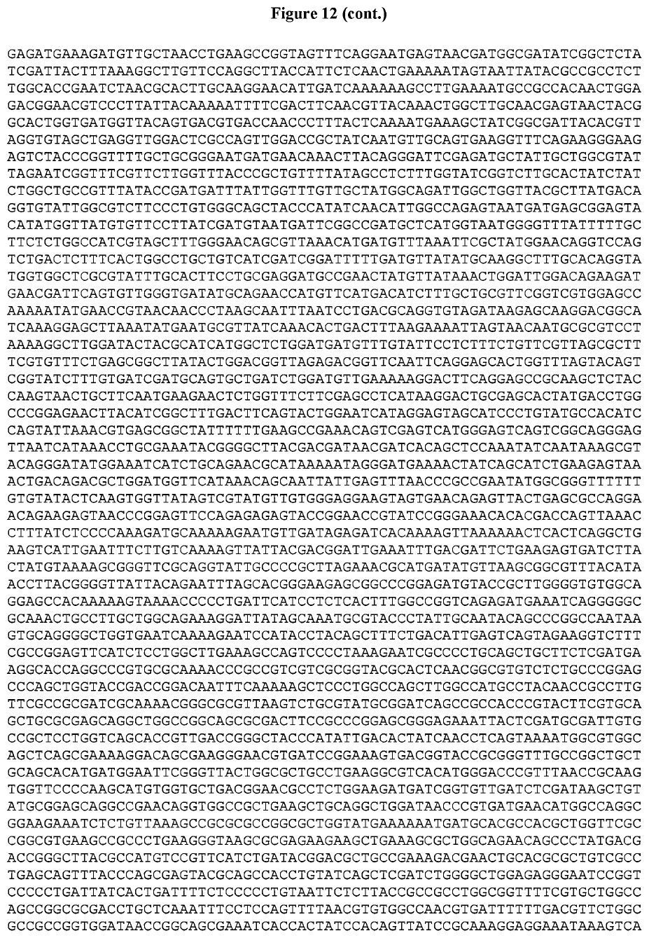

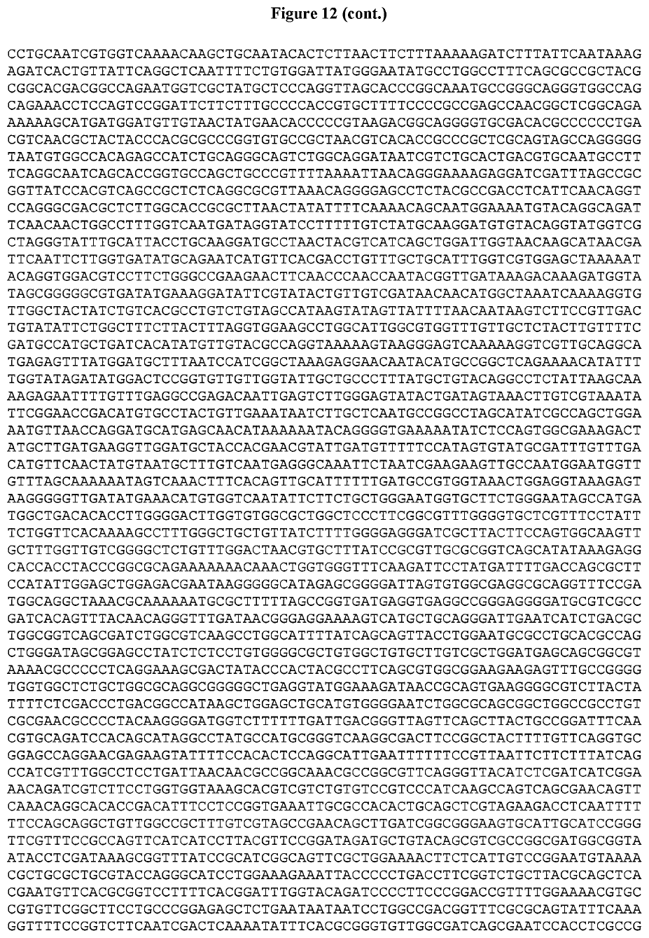

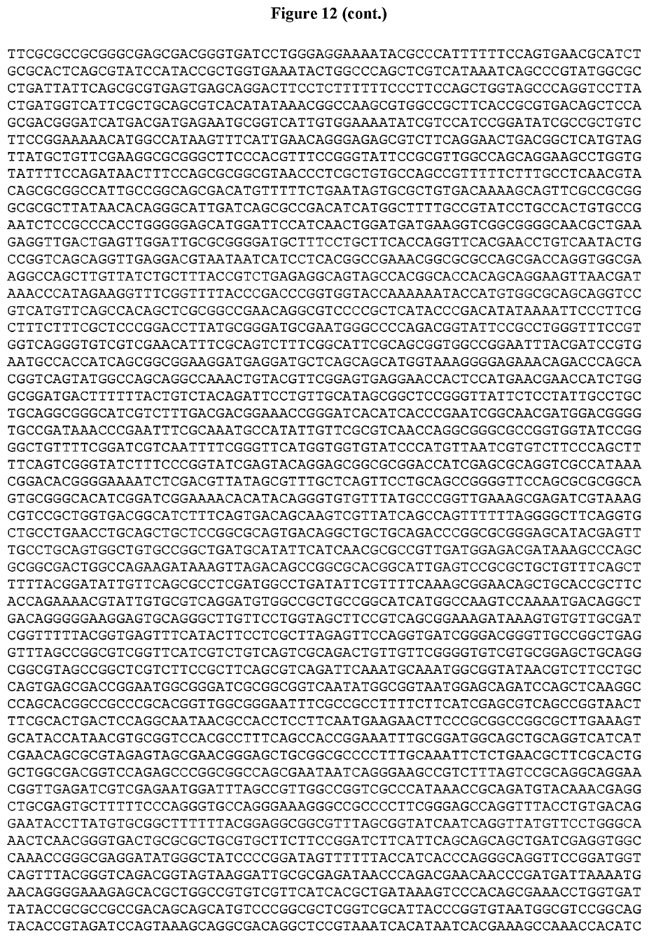

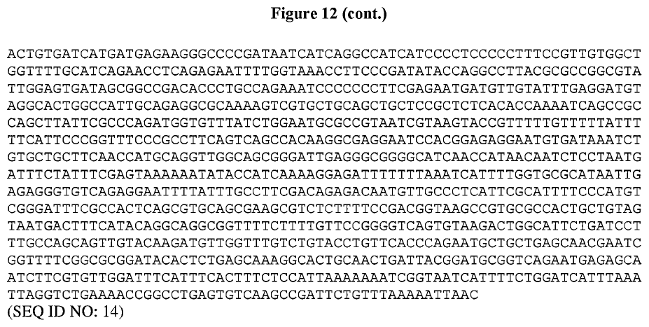

The resultant conjugative probiotic plasmid designated PB1 possessed an intact pemIK toxin-antitoxin system to ensure its stable maintenance in the bacterial population. To make it unstable, the pemK toxin gene was deleted by replacing it with the tetA gene. The resulting unstable probiotic plasmid was designated “PB1.1”. The conjugative probiotic plasmid PB1.1 is lost from bacteria in the absence of selection pressure. The Physical map and nucleotide sequences of the probiotic plasmid PB1.1 are shown in FIGS. 11 and 12, respectively. The PB1.1 sequence is set forth in SEQ ID NO: 13.

### Ability of Conjugative Probiotic Plasmid PB1.1 to Prevent Invasion by IncM, IncL, and IncC Plasmids in Bacteria.

The probiotic plasmid PB1.1 was then tested for its capacity to prevent invasion by AMR plasmids of IncM, IncL and IncC groups into bacteria using standard liquid mating experiments.

Briefly, donor and recipient bacteria were grown overnight into a 10 mL LB-Lennox broth medium with appropriate antibiotics. Cultures were washed twice with an equivalent volume of sterile saline solution (0.85% NaCl) and resuspended in 10 mL LB_Lennox broth. The OD₆₀₀ of each culture was adjusted to 2.0, and 1 mL of each donor and recipient cultures were mixed and taken into a 15 mL falcon tube. Mating mixtures were incubated at 37° C. for 20 h without shaking. Mating was ended by vortexing the mating mixtures for 10 sec. Mating mixtures were then serially diluted with saline and plated onto LB-Lennox agar plates containing sodium azide (100 mg/mL) and cefotaxime (8 mg/mL) to select transconjugants. Conjugation frequency is the ratio of the total number of transconjugants to the number of recipient bacteria added to the conjugation mixture. E. coli NH78Rf with IncM plasmid pJIBE401, IncL plasmid pJIE1335, or IncC plasmid pEc158 were used as donor bacteria, and sodium azide resistant bacteria J53Az and J53Az carrying probiotic plasmid PB1.1 were used as the recipient for each plasmid transfer.

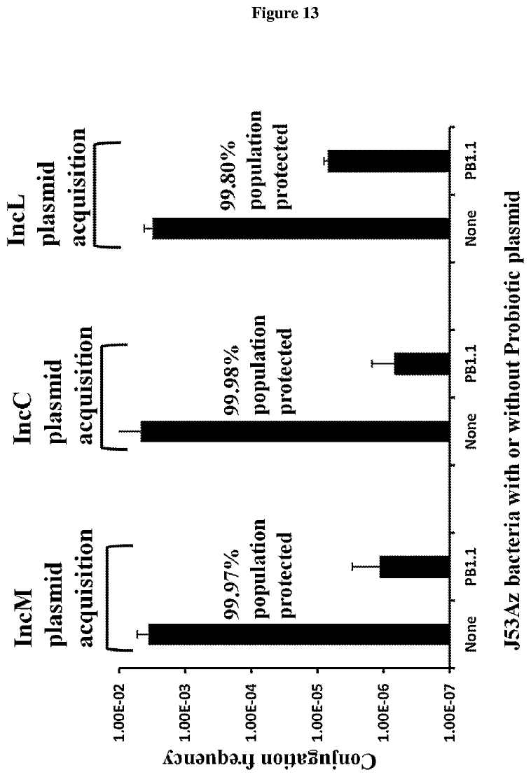

Antibiotic resistance IncM, IncL, and IncC plasmids can readily be transferred into recipient bacteria J53Az after 20 h liquid mating experiments, but the transfer of these plasmids into J53Az(pPB1.1) was significantly reduced after the same time of the mating (FIG. 13). Conjugative probiotic plasmid protected >99.9% of the recipient bacterial population from the invasion of different groups of AMR plasmids during mating experiments. This study showed that a single probiotic plasmid PB1.1 in the recipient bacteria could significantly prevent the acquisition of IncM, IncL, and IncC plasmids tested.

The characteristics of bacterial strains and plasmids used in this experiment are shown in Table 6.

TABLE 6
Characteristics of bacterial strains and plasmids

| | Bacteria/ | | |
|---|---|---|---|
| ID | Plasmid | Characteristics | Source/Reference |
| J53Az | Bacteria | Sodium azide resistance Escherichia coli K-12 strain | (Matsumura, |
| | | | Peirano & Pitout, |
| | | | 2018) |
| UB5201Rf | Bacteria | Rifampicin resistance derivative of E. coli UB5201 | (de la Cruz & |
| | | strain with rpoB mutation S512P | Grinsted, 1982) |
| NH78Rf | Bacteria | Rifampicin resistance derivative of E. coli ST131 | This study |
| | | clinical strain with rpoB mutation D516G | |
| pJIBE401 | Plasmid | Naturally occurring IncM plasmid carrying bla_{IMP-4} | (Espedido, |
| | | carbapenemase gene | Partridge & |
| | | | Iredell, 2008) |
| pJIMK45 | Plasmid | pJIBE401 derivative, ~28 kb MRR region replaced | (Kamruzzaman et |
| | | with tetA gene | al., 2017) |
| pEc158 | Plasmid | Naturally occurring IncC plasmid carrying bla_{IMP-4} | (Papagiannitsis et |
| | | carbapenemase gene | al., 2017) |
| pJIE1335 | Plasmid | Naturally occurring IncL plasmid carrying | (Agyekum et al., |
| | | bla_{OXA-48} carbapenemase gene | 2016; Fajardo- |
| | | | Lubian et al., |
| | | | 2019) |
| pPB1 | Plasmid | Probiotic plasmid, resistance to fosfomycin. The | This study |
| | | tetA gene of pJIMK45 was replaced with IncL_exc- | |
| | | IncC_exc-fosA3 gBlock. | |
| pPB1.1 | Plasmid | Probiotic plasmid, resistance to fosfomycin and | This study |
| | | tetracycline. The pemK toxin gene of pPB1 was | |
| | | replaced with tetA gene. | |
| pKM200 | Plasmid | Lambda red recombinase plasmid, chloramphenicol | (Murphy & |
| | | resistance | Campellone, |
| | | | 2003) |

### Example 5: Probiotic Plasmid PB1.1 Protects Mouse Microbiota from the Acquisition of Different AMR Plasmids

In this example, the inventors evaluated the ability of the conjugative probiotic plasmid PB31.1 to protect mouse gut microbiota from the acquisition of different AMR plasmid types described in Table 6 (including pJIBE401, pJIE1335, and pEc158) during incidental exposure to infected faeces through coprophagy.

Test mice were colonised with probiotic plasmid PB1.1, whereas control mice were fed normal feed with no supplement. All mice (i.e., test and control mice) were exposed to mice colonised with three different common AMR plasmid types. All mice were housed in groups of three as biological replicates within the experiment.

Sodium azide resistant E. coli J53Az carrying probiotic plasmid PB1.1 and Rifampicin resistant E. coli NH78Rf carrying different AMR plasmids were provided to mice in 8% sucrose water.

Test and control mice were co-housed for 8 h each day with AMR-colonised mice and then separated for 16 hours, with or without PB1.1 in 8% sucrose water. AMR plasmid colonised mice were co-housed with either probiotic plasmid colonised mice or control mice for a total of 96 h (4 days) for this experiment.

Fresh faeces were collected and examined on specific antibiotic-containing ChromAgar plates (with CTX 8 μg/mL supplementation for AMR plasmids or 200 μg/mL of Fosfomycin for probiotic plasmid) to measure colonisation, using serial dilution to quantify.

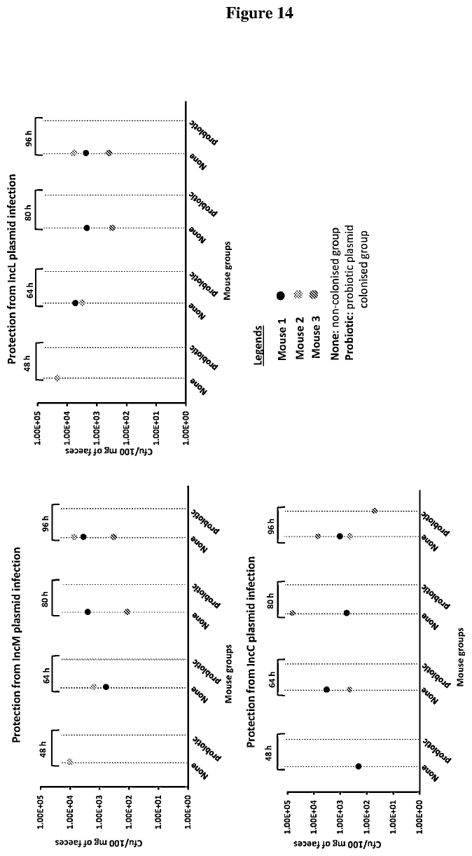

AMR plasmids appeared in different mice in the non-colonised control group after a total of 48 h of exposure to infected mice, with all control mice becoming AMR colonised within 96 h (4 days equivalent) of exposure. By contrast, no AMR plasmids were detected in any mice receiving probiotic plasmids until 80 h after initial exposure (FIG. 14). One mouse from the IncC group showed very low level of AMR plasmid infection in the probiotic group after 96 h of exposure. These data indicate strong protection against acquisition and colonisation by AMR plasmids after an extensive time of being co-housed with mice highly colonised with AMR plasmids.

PCR conducted at 96 h revealed AMR plasmid DNA in all mice (data not shown), confirming that AMR plasmids were being ingested. These data suggest that ingested AMR plasmids were unable to colonise to a level where they could be readily detected by standard culture methods in those mice that received overnight dosing with probiotic plasmid therapy, even after regular exposure and ingestion of common AMR plasmid types.

As a test of protection, both probiotic-colonised (AMR culture negative but PCR positive) and control mice (AMR culture positive after exposure) were treated with cefotaxime (CTX) antibiotic to select for the mouse gut bacterial populations carrying AMR plasmids, after 120 h of exposure to AMR colonised mice. Fresh faeces were collected and examined on ChromAgar plates with CTX antibiotic using serial dilutions.

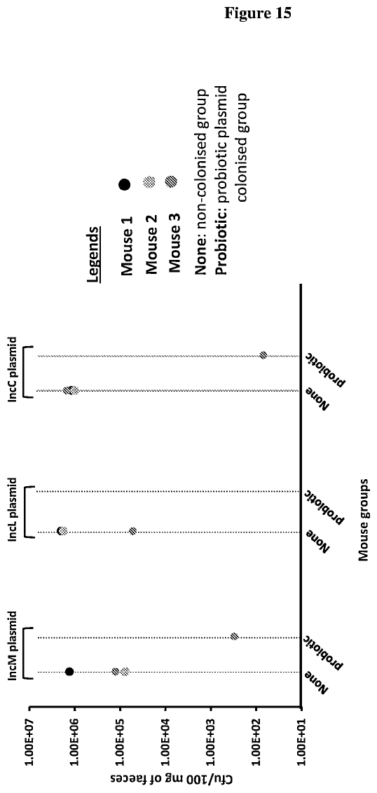

A high level of AMR (CTX-resistant) bacteria was detected in non-treated control mice (FIG. 15). By contrast, AMR bacteria were detected in only one probiotic plasmid treated mouse exposed to the IncM AMR plasmid group and one mouse exposed to the IncC AMR plasmid group, and at very low levels. Since PCR evidence of the AMR plasmid DNA was found in all mice, this strongly supports that probiotic plasmid protects the gut environment from AMR plasmids.

All publications discussed and/or referenced herein are incorporated herein in their entirety.

### REFERENCES

Agyekum, A., Fajardo-Lubian, A., Ai, X., Ginn, A. N., Zong, Z., Guo, X., et al. (2016). Predictability of Phenotype in Relation to Common beta-Lactam Resistance Mechanisms in Escherichia coli and Klebsiella pneumoniae. J Clin Microbiol 54, 1243-50.
De La Cruz, F. & Grinsted, J. (1982). Genetic and molecular characterization of Tn21, a multiple resistance transposon from R100.1. J Bacteriol 151, 222-28.
Espedido, B. A., Partridge, S. R. & Iredell, J. R. (2008). bla_{IMP-4} in different genetic contexts in Enterobacteriaceae isolates from Australia. Antimicrob Agents Chemother 52, 2984-7.
Fajardo-Lubian, A., Ben Zakour, N. L., Agyekum, A., Qi, Q. & Iredell, J. R. (2019). Host adaptation and convergent evolution increases antibiotic resistance without loss of virulence in a major human pathogen. PLoS Pathog 15, e1007218.
Humbert, M., Huguet, K. T., Coulombe, F. & Burrus, V. (2019). Entry Exclusion of Conjugative Plasmids of the IncA, IncC, and Related Untyped Incompatibility Groups. J Bacteriol 201.
Kamruzzaman, M., Shoma, S., Thomas, C. M., Partridge, S. R. & Iredell, J. R. (2017). Plasmid interference for curing antibiotic resistance plasmids in vivo. PLoS One 12, e0172913.
Matsumura, Y., Peirano, G. & Pitout, J. D. D. (2018). Complete genome sequence of Escherichia coli J53, an azide-resistant laboratory strain used for conjugation experiments. Genome Announc 6.
Murphy, K. C. & Campellone, K. G. (2003). Lambda Red-mediated recombinogenic engineering of enterohemorrhagic and enteropathogenic E. coli. BMC Mol Biol 4, 11.
Papagiannitsis, C. C., Kutilova, I., Medvecky, M., Hrabak, J. & Dolejska, M. (2017). Characterization of the Complete Nucleotide Sequences of IncA/C. Antimicrob Agents Chemother 61.

1. A recombinant plasmid comprising entry exclusion systems (EES) from a plurality of plasmid variants, where the plasmid variants comprise a gene conferring a trait of interest.

2. The recombinant plasmid of claim 1, wherein the trait of interest is a trait which is undesirable in a bacterial population.

3. The recombinant plasmid of claim 2, wherein the trait of interest is antimicrobial resistance (AMR), bacterial virulence, or heavy metal resistance.

4. The recombinant plasmid of claim 1, wherein the recombinant conjugative plasmid comprises EES from at least three plasmid variants comprising a gene conferring the trait of interest.

5. The recombinant plasmid of any one of claims 1 to 4, wherein the EES are from different incompatibility groups.

6. The recombinant plasmid of any one of claims 1 to 4, wherein the EES are from the same incompatibility group.

7. The recombinant plasmid of any one of claims 1 to 6, wherein one or more of the EES are from a plasmid variant from an incompatibility group (Inc) selected from IncF, IncI, IncA, IncC, IncL, IncM, IncX, IncP, IncN and IncH.

8. The recombinant plasmid of claim 7, wherein one or more of the EES are from plasmid variants of incompatibility group F (IncF).

9. The recombinant plasmid of claim 8, comprising one or more of:
an EES comprising a polynucleotide sequence which is at least 80% identical to the sequence set forth in SEQ ID NO: 1 (traS_F variant);
an EES comprising a polynucleotide sequence which is at least 80% identical to the sequence set forth in SEQ ID NO: 2 (traS-R100 variant); and/or
an EES comprising a polynucleotide sequence which is at least 80% identical to the sequence set forth in SEQ ID NO: 3 (traS_SLT variant).

10. The recombinant plasmid of claim 8 or 9, comprising one or more of:
an EES comprising a polynucleotide sequence set forth in SEQ ID NO: 1 (traS_F variant);
an EES comprising a polynucleotide sequence set forth in SEQ ID NO: 2 (traS_R100 variant); and/or
an EES comprising a polynucleotide sequence set forth in SEQ ID NO: 3 (traS_SLT variant).

11. The recombinant plasmid of any one of claims 8 to 12, comprising the traS_F variant EES, the traS_R100 variant EES and the traS_SLT variant EES.

12. The recombinant plasmid of any one of claims 1 to 10, comprising one or more EES from plasmid variants of incompatibility group L (IncL), incompatibility group C (IncC), incompatibility group M (IncM), incompatibility group A (IncA) and any combinations thereof.

13. The recombinant plasmid of claim 12, comprising one or more of:
an EES comprising a polynucleotide sequence which is at least 80% identical to the sequence set forth in SEQ ID NO: 9 (excL variant);
an EES comprising a polynucleotide sequence which is at least 80% identical to the sequence set forth in SEQ ID NO: 10 (excC variant);
an EES comprising a polynucleotide sequence which is at least 80% identical to the sequence set forth in SEQ ID NO: 11 (excM variant); and/or
an EES comprising a polynucleotide sequence which is at least 80% identical to the sequence set forth in SEQ ID NO: 12 (excA variant).

14. The recombinant plasmid of claim 12 or 13, comprising one or more of:
an EES comprising a polynucleotide sequence set forth in SEQ ID NO: 9 (excL variant);
an EES comprising a polynucleotide sequence set forth in SEQ ID NO: 10 (excC variant);
an EES comprising a polynucleotide sequence set forth in SEQ ID NO: 11 (excM variant); and/or
an EES comprising a polynucleotide sequence set forth in SEQ ID NO: 12 (excA variant).

15. The recombinant plasmid of any one of claims 1 to 14, wherein one or more of the EES are operably-linked to a constitutive promoter, optionally wherein the constitutive promoter is selected from a promoter that is native to the EES or a strong integron promoter (PcS).

16. The recombinant plasmid of any one of claims 1 to 14, wherein one or more of the EES are operably-linked to an inducible promoter, optionally wherein the inducible promoter is a L-arabinose inducible promoter.

17. The recombinant plasmid of any one of claims 1 to 16, wherein each EES is operably-linked to a separate promoter.

18. The recombinant plasmid of any one of claims 1 to 16, wherein the one or more EES are operably-linked to the same promoter.

19. The recombinant plasmid of claims 1 to 18, further comprising multiple plasmid replication incompatibility (Inc) control regions (inc-control), and/or a selectable marker or gene.

20. The recombinant conjugative plasmid of claim 19, wherein:
the Inc control regions are from plasmid variants from an Inc group selected from IncF, IncI, IncA, IncC, IncL, IncM, IncX, IncP, IncN and IncH;
the Inc control region(s) and the EES are from the same Inc group(s);
one or more of the Inc control regions are from plasmid variants of IncF;
one or more of the Inc control regions are from plasmid variants of IncA, IncC, IncL and/or IncM; and/or
the selectable marker or gene is an antibiotic resistance gene.

21. A bacterial cell comprising a recombinant plasmid of any one of claims 1 to 20.

22. The bacterial cell of claim 21, wherein the bacteria is a probiotic bacteria.

23. The bacterial cell of claim 22, wherein the probiotic bacteria is selected from a group including but not limited to Escherichia, Klebsiella, other members of the Enterobacteriaceae family, Lactobacillus, Bifidobacteria, Streptococcus, Lactococcus, Enterococcus, Propionibacterium, Faecalibacterium, Pediococcus, Ruminococcus, Megasphaera, Bacillus and suitable members of the Pseudomonadaceae and Vibrionacecae family.

24. A composition comprising one or more of viable bacterial cells according to any one of claims 21 to 23.

25. The composition of claim 24, wherein the viable bacterial cells are lyophilised and/or formulated in a food or beverage product.

26. A method of preventing or reducing the acquisition and/or spread of a plasmid conferring a trait of interest in a population of bacteria, said method comprising introducing to the population one or more bacterial cells of any one of claims 21 to 23 or a composition comprising same of claim 24 or 25.

27. The method of claim 26, wherein the trait of interest is:
(i) antimicrobial resistance (AMR), and one or more of the bacterial cells introduced to the population comprises a recombinant conjugative plasmid of any one of claims 1 to 30 comprising entry exclusion systems (EES) from a plurality of plasmid variants conferring AMR; or
(ii) bacterial virulence, and one or more of the bacterial cells introduced to the population comprises a recombinant conjugative plasmid of any one of claims 1 to 20 comprising entry exclusion systems (EES) from a plurality of plasmid variants conferring bacterial virulence.

28. The method of claim 26 or 27, wherein the population of bacteria is a population of gut bacteria and the method comprises administering the one or more bacterial cells or composition comprising same to a subject in need thereof.

29. The method according to claim 28, wherein the subject is a human or non-human animal.

30. The method of claim 26 or 27, wherein:
(i) the population of bacteria is a population of bacteria which colonise plants and the method comprises contacting the plant or soil in which the plant is growing with the one or more bacterial cells or composition comprising same; or
(ii) the population of bacteria is present in an environment selected from a healthcare environment, a soil environment, an environment comprising a water source, an environment comprising waste water, an environment comprising industrial waste, an environment comprising agricultural waste, an environment comprising sewerage and/or an environment comprising biosolids, and the method comprises introducing the one or more bacterial cells or composition comprising same to the environment.

31. A food product or beverage comprising one or more bacterial cells of any one of claims 21 to 23 or a composition of claim 24 or 25.

32. The food product of claim 31, wherein the food product is a dairy product.


SEQUENCE LISTING (14 sequences)

SEQ ID NO: 1 - DNA, 522 bp, synthetic construct
  misc_feature 1..522: note = F plasmid variant entry exclusion system designated traS_R100
  source 1..522: mol_type = other DNA
atgattacacaacaaattatcagtagtgagttggaagttttaaagaaacatattgactcaggagatattagaattccgtcactatggcagggattaaaacccgggcttatcatcatggggtggatgattttttgtcccttgctaatgtcatttctaataacacaaaagacaagtgaaacactcactgctgtgttagccggaggatggcttggacttattattctgtttattgtggcaaggattagaatgttatatttctctctgccggaagaattcctaaagacatcatccgtaatgagggttatatcatcaaaacttaaagtgtatttcattgtgtatatgggagttatatttttatggtcttttttaggtggagggattatatacggttttggcgcaattttagtgacggttattatggcgttcttgattcaattagatatcggtagatatcaatttgttggtgttattgatgcaatcaatagttacgtcaaaaacaagaagttatcaagagtaaaataa

SEQ ID NO: 2 - DNA, 480 bp, synthetic construct
  misc_feature 1..480: note = F plasmid variant entry exclusion system designated traS_F
  source 1..480: mol_type = other DNA
atgagatgtttaacccatattactctggtaacggtgatacagtttatcgcctgttatctggctagttgggggaatgccgaaacaatttttatgctgttcttcatagtactatggcagggactgtttatatggttgttctctcaaatacgaaaaaaacggaatgtgtcggatgagtttaagtttagtaaaggcgtttggtatataacgatgccagtatcttccttactctctccattattaagcctgatggtttttatcattggtacactatacgaattaaggcgagttagtggttgtgtaagcgtaagagaatggatgcaatctcaggtaaatgaacaaactaatgaagatttgcatctggattttgataatatggattttcatcgtactaatcctgcaacagggttacctatgtcaggtattggtgtggatatcggtggtaattcatatggttatagttcaaaacgatatgatgagtga

SEQ ID NO: 3 - DNA, 495 bp, synthetic construct
  misc_feature 1..495: note = F plasmid variant entry exclusion system designated traS_SLT
  source 1..495: mol_type = other DNA
gtgggaatgaaaaatttagctcagattgttctggtgacagtggtacagtttattgcctgttatctagctgagtggggagtcgctgaaacagggattatcctgttatttgtcctgctgtggcaggggctgtttatctggttatttattcaaattcgtaaaaaaaataacatatctgatgagttcaagtttagtaaaggaatttggtatgtaattatgcccgtatgctcattactctcgccattattaagtttaatgatttttatcggtggaacattatatgaattaagacgagtaagtggttgtataagcataaaagagtgggtgaaatgtcagcttgatgaccaatatgatgaagatagggggctggattttgaatctgttgagtatcgtcaaacgacttactacaacccctcaactggatatcctatgcatggaggagttgattcggctggtaatacttttgggtctcgctggcaggataataatgacaggtaa

SEQ ID NO: 4 - DNA, 690 bp, synthetic construct
  misc_feature 1..690: note = F plasmid variant entry exclusion system designated traS_R100 with native promoter, RBS and restriction sites
  source 1..690: mol_type = other DNA
gcagaatcaccataaaacagaggcattatctcagaataataaatataatgaagaaaagtcggctcaggaaagaatgcccggggctgacagtccacaggaactgatgaaaagggcaaaggaatatcaggataagtataagcaataatgctgtttttgggagatgataatgagatgtttaacccatattactctggtaacggtgatacagtttatcgcctgttatctggctagttgggggaatgccgaaacaatttttatgctgttcttcatagtactatggcagggactgtttatatggttgttctctcaaatacgaaaaaaacggaatgtgtcggatgagtttaagtttagtaaaggcgtttggtatataacgatgccagtatcttccttactctctccattattaagcctgatggtttttatcattggtacactatacgaattaaggcgagttagtggttgtgtaagcgtaagagaatggatgcaatctcaggtaaatgaacaaactaatgaagatttgcatctggattttgataatatggattttcatcgtactaatcctgcaacagggttacctatgtcaggtattggtgtggatatcggtggtaattcatatggttatagttcaaaacgatatgatgagtgaatcttaatttgtatattatgacgttttattcaatatgaaggaac

SEQ ID NO: 5 - DNA, 774 bp, synthetic construct
  misc_feature 1..774: note = F plasmid variant entry exclusion system designated traS_F with native promoter, RBS and restriction sites
  source 1..774: mol_type = other DNA
aaagggcagtattctgaactacaaaatcatcataaaacagaagcattaactcagaataataaatataatgaagaaaagctggctcaggaaagaatacctggtgctgatagtccgaaagagcttttagagaaagcaaagagttaccagcataaagaataatcataacatgttgggtagggtatggagagaccatgattacacaacaaattatcagtagtgagttggaagttttaaagaaacatattgactcaggagatattagaattccgtcactatggcagggattaaaacccgggcttatcatcatggggtggatgattttttgtcccttgctaatgtcatttctaataacacaaaagacaagtgaaacactcactgctgtgttagccggaggatggcttggacttattattctgtttattgtggcaaggattagaatgttatatttctctctgccggaagaattcctaaagacatcatccgtaatgagggttatatcatcaaaacttaaagtgtatttcattgtgtatatgggagttatatttttatggtcttttttaggtggagggattatatacggttttggcgcaattttagtgacggttattatggcgttcttgattcaattagatatcggtagatatcaatttgttggtgttattgatgcaatcaatagttacgtcaaaaacaagaagttatcaagagtaaaataaaagatattagagagtaaatatatgatgaaaacaaaaaaactaatgatggttgcactggtca

SEQ ID NO: 6 - DNA, 691 bp, synthetic construct
  misc_feature 1..691: note = F plasmid variant entry exclusion system designated traS_SLT with native promoter, RBS and restriction sites
  source 1..691: mol_type = other DNA
attctggcctgcaaaaacagtataaaacagaagcactatctcagaataataaatataatgaagaaaaggctgcacaggaaaggatgcctggcgcggatagtcctgaagaactgatgaagagggcaaaagagtatcaggataagcacaaacagtaaggcgatatccgggaggtgggaatgaaaaatttagctcagattgttctggtgacagtggtacagtttattgcctgttatctagctgagtggggagtcgctgaaacagggattatcctgttatttgtcctgctgtggcaggggctgtttatctggttatttattcaaattcgtaaaaaaaataacatatctgatgagttcaagtttagtaaaggaatttggtatgtaattatgcccgtatgctcattactctcgccattattaagtttaatgatttttatcggtggaacattatatgaattaagacgagtaagtggttgtataagcataaaagagtgggtgaaatgtcagcttgatgaccaatatgatgaagatagggggctggattttgaatctgttgagtatcgtcaaacgacttactacaacccctcaactggatatcctatgcatggaggagttgattcggctggtaatacttttgggtctcgctggcaggataataatgacaggtaataagaaaactatggttggtaaataaa

SEQ ID NO: 7 - DNA, 2011 bp, synthetic construct
  misc_feature 1..2011: note = pJIMK_traS_3 comprising F plasmid variant entry exclusion systems designated traS_R100, and traS_SLT with native promoter, and traS_F with integron promoter Pc, RBS and flanking HindIII and XbaI restriction sites
  source 1..2011: mol_type = other DNA
gcgaagcttgcagaatcaccataaaacagaggcattatctcagaataataaatataatgaagaaaagtcggctcaggaaagaatgcccggggctgacagtccacaggaactgatgaaaagggcaaaggaatatcaggataagtataagcaataatgctgtttttgggagatgataatgagatgtttaacccatattactctggtaacggtgatacagtttatcgcctgttatctggctagttgggggaatgccgaaacaatttttatgctgttcttcatagtactatggcagggactgtttatatggttgttctctcaaatacgaaaaaaacggaatgtgtcggatgagtttaagtttagtaaaggcgtttggtatataacgatgccagtatcttccttactctctccattattaagcctgatggtttttatcattggtacactatacgaattaaggcgagttagtggttgtgtaagcgtaagagaatggatgcaatctcaggtaaatgaacaaactaatgaagatttgcatctggattttgataatatggattttcatcgtactaatcctgcaacagggttacctatgtcaggtattggtgtggatatcggtggtaattcatatggttatagttcaaaacgatatgatgagtgacatcataaaacagaagcattaactcagaataataaatataatgaagaaaagctggctcaggaaagaatacctggtgctgatagtccgaaagagcttttagagaaagcaaagagttaccagcataaagaataatcataacatgttgggtagggtatggagagaccatgattacacaacaaattatcagtagtgagttggaagttttaaagaaacatattgactcaggagatattagaattccgtcactatggcagggattaaaacccgggcttatcatcatggggtggatgattttttgtcccttgctaatgtcatttctaataacacaaaagacaagtgaaacactcactgctgtgttagccggaggatggcttggacttattattctgtttattgtggcaaggattagaatgttatatttctctctgccggaagaattcctaaagacatcatccgtaatgagggttatatcatcaaaacttaaagtgtatttcattgtgtatatgggagttatatttttatggtcttttttaggtggagggattatatacggttttggcgcaattttagtgacggttattatggcgttcttgattcaattagatatcggtagatatcaatttgttggtgttattgatgcaatcaatagttacgtcaaaaacaagaagttatcaagagtaaaataaaacaaaaacagtataaaacagaagcactatctcagaataataaatataatgaagaaaaggctgcacaggaaaggatgcctggcgcggatagtcctgaagaactgatgaagagggcaaaagagtatcaggataagcacaaacagtaaggcgatatccgggaggtgggaatgaaaaatttagctcagattgttctggtgacagtggtacagtttattgcctgttatctagctgagtggggagtcgctgaaacagggattatcctgttatttgtcctgctgtggcaggggctgtttatctggttatttattcaaattcgtaaaaaaaataacatatctgatgagttcaagtttagtaaaggaatttggtatgtaattatgcccgtatgctcattactctcgccattattaagtttaatgatttttatcggtggaacattatatgaattaagacgagtaagtggttgtataagcataaaagagtgggtgaaatgtcagcttgatgaccaatatgatgaagatagggggctggattttgaatctgttgagtatcgtcaaacgacttactacaacccctcaactggatatcctatgcatggaggagttgattcggctggtaatacttttgggtctcgctggcaggataataatgacaggtaataagatctagacgc

SEQ ID NO: 8 - DNA, 1879 bp, synthetic construct
  misc_feature 1..1879: note = pJIMK_traS_3 with araBAD promoter
  source 1..1879: mol_type = other DNA
gcgtctagatttttgggagatgataatgagatgtttaacccatattactctggtaacggtgatacagtttatcgcctgttatctggctagttgggggaatgccgaaacaatttttatgctgttcttcatagtactatggcagggactgtttatatggttgttctctcaaatacgaaaaaaacggaatgtgtcggatgagtttaagtttagtaaaggcgtttggtatataacgatgccagtatcttccttactctctccattattaagcctgatggtttttatcattggtacactatacgaattaaggcgagttagtggttgtgtaagcgtaagagaatggatgcaatctcaggtaaatgaacaaactaatgaagatttgcatctggattttgataatatggattttcatcgtactaatcctgcaacagggttacctatgtcaggtattggtgtggatatcggtggtaattcatatggttatagttcaaaacgatatgatgagtgaaaaacagaagcattaactcagaataataaatataatgaagaaaagctggctcaggaaagaatacctggtgctgatagtccgaaagagcttttagagaaagcaaagagttaccagcataaagaataatcataacatgttgggtagggtatggagagaccatgattacacaacaaattatcagtagtgagttggaagttttaaagaaacatattgactcaggagatattagaattccgtcactatggcagggattaaaacccgggcttatcatcatggggtggatgattttttgtcccttgctaatgtcatttctaataacacaaaagacaagtgaaacactcactgctgtgttagccggaggatggcttggacttattattctgtttattgtggcaaggattagaatgttatatttctctctgccggaagaattcctaaagacatcatccgtaatgagggttatatcatcaaaacttaaagtgtatttcattgtgtatatgggagttatatttttatggtcttttttaggtggagggattatatacggttttggcgcaattttagtgacggttattatggcgttcttgattcaattagatatcggtagatatcaatttgttggtgttattgatgcaatcaatagttacgtcaaaaacaagaagttatcaagagtaaaataatctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttaggaggagaccatgggaatgaaaaatttagctcagattgttctggtgacagtggtacagtttattgcctgttatctagctgagtggggagtcgctgaaacagggattatcctgttatttgtcctgctgtggcaggggctgtttatctggttatttattcaaattcgtaaaaaaaataacatatctgatgagttcaagtttagtaaaggaatttggtatgtaattatgcccgtatgctcattactctcgccattattaagtttaatgatttttatcggtggaacattatatgaattaagacgagtaagtggttgtataagcataaaagagtgggtgaaatgtcagcttgatgaccaatatgatgaagatagggggctggattttgaatctgttgagtatcgtcaaacgacttactacaacccctcaactggatatcctatgcatggaggagttgattcggctggtaatacttttgggtctcgctggcaggataataatgacaggtaaaagcttgcg

SEQ ID NO: 9 - DNA, 654 bp, synthetic construct
  source 1..654: mol_type = other DNA
atgaaaggatattcgtatactgttgtcgataacaacatggctaaatcaaaaggtgttggctactatctgtcacgcctgtctgtagccataagtatagttattttaacaataagtcttccgttgactgtatattctggctttcttactttaggtggaagcctggcattggcgtggtttgttgctctacttgttttcgatgccatgctgatcacatatgttgtacgccaggtaaaaagtaagggagtcaaaaaggtcgttgcaggcatgagagtttatggatgctttaatccatcggctaaagaggaacaatacatgccggctcagaaaacatattttggtatagatatggactccggtgttgttggtattgctgccctttatgctgtacaggcctctattaagcaaaagagaattttgtttgaggccgagacaattgagtcttgggagtatactgatagtaaacttgtcgtaaatattcggaaccgacatgtgcctactgttgaaataatcttgctcaatgccggcctagcatatcgccagctggaaatgttaaccaggatgcatgagcaacataaaaaatacaggggtgaaaaatatctccagtggcgaaagactatgcttgatgaaggttggatgctaccacgaacgtattga

SEQ ID NO: 10 - DNA, 363 bp, synthetic construct
  source 1..363: mol_type = other DNA
atgaaacatgtggtcaatattcttctgctgggaatggtgcttctgggaatagccatgatggctgacacaccttggggacttggtgtggcgctggctcccttcggcgtttggggtgctcgtttcctatttctggttcacaaaagcctttgggctgctgttatcttttggggagggatcgcttacttccagtggcaagttgctttggttgtcggggctctgtttggactaacgtgctttatccgcgttgcgcggtcagcatataaagaggcaccacctacccggcgcagaaaaaaacaaactggtgggtttcaagattcctatgattttgaccagcgcttccatattggagctggagacgaataa

SEQ ID NO: 11 - DNA, 654 bp, synthetic construct
  source 1..654: mol_type = other DNA
atgaatgcgttatcaaacactgactttaagaaaattagtaacaatgcgcgtcctaaaaggcttggatactacgcatcatggctctggatgatgtttgtattcctctttctgttcgttagcgctttcgtgtttctgagcggcttatactggacggttagagacggttcaattcaggagcactggtttagtacagtcggtatctttgtgatcgatgcagtgctgatctggatgttgaaaaaggacttcaggagccgcaagctctaccaagtaactgcttcaatgaagaactctggtttcttcgagcctcataaggactgcgagcactatgacctggcccggagaacttacatcggctttgacttcagtactggaatcataggagtagcatccctgtatgccacatccagtattaaacgtgagcggctattttttgaagccgaaacagtcgagtcatgggagtcagtcggcagggagttaatcataaacctgcgaaatacggggcttacgacgataacgatcacagctccaaatatcaataaagcgtacagggatatggaaatcatctgcagaacgcataaaaatagggatgaaaactatcagcatctgaagagtaaactgacagacgctggatggttcataaacagcaattattga

SEQ ID NO: 12 - DNA, 363 bp, synthetic construct
  source 1..363: mol_type = other DNA
atgaaacatgtggtcaatattcttctgctgggtgttgtgcttctgggggtcgctatgatggctgatacaccttgggggcttggtgttgccctggctccttttgccgtttggggggctcgtttcctgtttttcattcacagaagtatttgggctgctgttatcttctggggagggatcgcttacttccagtggcaagttgctttggctgtcggggctctgtttgggctaacatggtttatcggtgctgcgcggtcagcatataaagaagctccgccaattaggcacagaaaaaaacatgctggtggatttcaaaactcctatgattttgaccagcgctaccatattggagctggagacgaataa

SEQ ID NO: 13 - DNA, 2176 bp, synthetic construct
  source 1..2176: mol_type = other DNA
gctcttggcaccgcgcttaactatattttcaaaacagcaatggaaaatgtacaggcagattcaacaactggcctttggtcaatgataggtatcctttttgtctatgcaaggatgtgtacaggtatggtcgctagggtatttgcattacctgcaaggatgcctaactacgtcatcagctggattggtaacaagcataacgattcaattcttggtgatatgcagaatcatgttcacgacctgtttgctgcatttggtcgtggagctaaaaatacaggtggacgtccttctgggccgaagaacttcaacccaaccaatacggttgataaagacaaagatggtatagcgggggcgtgatatgaaaggatattcgtatactgttgtcgataacaacatggctaaatcaaaaggtgttggctactatctgtcacgcctgtctgtagccataagtatagttattttaacaataagtcttccgttgactgtatattctggctttcttactttaggtggaagcctggcattggcgtggtttgttgctctacttgttttcgatgccatgctgatcacatatgttgtacgccaggtaaaaagtaagggagtcaaaaaggtcgttgcaggcatgagagtttatggatgctttaatccatcggctaaagaggaacaatacatgccggctcagaaaacatattttggtatagatatggactccggtgttgttggtattgctgccctttatgctgtacaggcctctattaagcaaaagagaattttgtttgaggccgagacaattgagtcttgggagtatactgatagtaaacttgtcgtaaatattcggaaccgacatgtgcctactgttgaaataatcttgctcaatgccggcctagcatatcgccagctggaaatgttaaccaggatgcatgagcaacataaaaaatacaggggtgaaaaatatctccagtggcgaaagactatgcttgatgaaggttggatgctaccacgaacgtattgatgtttttccatagtgtatgcgatttgtttgacatgttcaactatgtaatgctttgtcaatgagggcaaattctaatcgaagaagttgccaatggaatggttgtttagcaaaaaatagtcaaactttcacagttgcattttttgatgccgtggtaaactggaggtaaagagtaagggggttgatatgaaacatgtggtcaatattcttctgctgggaatggtgcttctgggaatagccatgatggctgacacaccttggggacttggtgtggcgctggctcccttcggcgtttggggtgctcgtttcctatttctggttcacaaaagcctttgggctgctgttatcttttggggagggatcgcttacttccagtggcaagttgctttggttgtcggggctctgtttggactaacgtgctttatccgcgttgcgcggtcagcatataaagaggcaccacctacccggcgcagaaaaaaacaaactggtgggtttcaagattcctatgattttgaccagcgcttccatattggagctggagacgaataagggggcatagagcggggattagtgtggcgaggcgcaggtttccgatggcaggctaaacgcaaaaaatgcgctttttagccggtgatgaggtgaggccgggaggggatgcgtcgccgatcacagtttacaacagggtttgataacgggaggaaaagtcatgctgcagggattgaatcatctgacgctggcggtcagcgatctggcgtcaagcctggcattttatcagcagttacctggaatgcgcctgcacgccagctgggatagcggagcctatctctcctgtggggcgctgtggctgtgcttgtcgctggatgagcagcggcgtaaaacgccccctcaggaaagcgactatacccactacgccttcagcgtggcggaagaagagtttgccggggtggtggctctgctggcgcaggcgggggctgaggtatggaaagataaccgcagtgaaggggcgtcttactattttctcgaccctgacggccataagctggagctgcatgtggggaatctggcgcagcggctggccgcctgtcgcgaacgcccctacaaggggatggtcttttttgattgacgggttagttcagcttactgccggatttcaacgtgcagatccacagc

SEQ ID NO: 14 - DNA, 62702 bp, synthetic construct
  source 1..62702: mol_type = other DNA
aggggataaaatgattcagtcacgtaataaatatctgcaatttatgctggttctgctggcagcctgggccatagcctggggggccctgtttgtcatggggcagaccgttctgctctatgggctggggaaaaatacgctgttcttcattcatggtgctgttctggtgtacctgctgtgtgtgttcctggtttaccgccgctgggtcgcaccgctaccggttgttggccagctgcgtaacgttggcgcaccgtggctggtcggtgcgatggccgtggtgtatgtcggagaatttctgctgggtaaagtgctggacctgcccgctgagccgtttatgacgaaactttttgccgataaatccatacccgacgtgatcctgacgttgctggccatctttatccttgcaccgctgaatgaggaaacgctgttccgggggatcatgctgaacgtcttccgttcacggtaccgctggacgatgtggctgggggcgctgataacgtcattgctgttcgtcgccgcgcacagccagtatcagaacctgctgaccctggcagaactgttcctggtggggttgattacatcagctgcccggatcagaagcggcggcctgctgctgccggtattgctgcatatggaagcaaccgcgctgggtttactgtttggctgaaagttatatttttattaaacattgcgcgttaaagcctggtatgtttttttagtggatgttatatttaaatataacttttatggaggtgaacaatgcataccacccgactgaagaaggttggcggctcagttatgctgaccgttccaccggcactgctgaatgcgctgtcgctgggtacagataatgaagttggcatggtcattgataatggccggctgattgtggagccgcacagacgcccgcagtattcactggctgagctactagcgcagtgcgatccgaatgctgaaatctcggctgaagaacgtgaatggctggatgcgccggtggctggtcaggaggaaatctgacatggaaagaggggaaatctggcttgtctcgcttgaccctaccgcaggtcatgagcagcagagcctctacgccgacctcattcaacaggtccagggcgacacggataactgtattaggctgcaactttatcatgcttgacactttatcactgataaacataatatgtccaccaacttatcagtgataaaggatccgtgagttctatcggaccagcggaggctggtccggaggccagcggaggctggtccggaggccagacgtgaaacccaacagacccctgatcgtaatactgtgcactgtcgcgctcgacgctgtcggcatcggcctgattatgccggtgctgccgggcctcctgcgagatctggttcactcgaacgacgtcaccgcccactatggcattttgctggcgctgtatgcgttgatgcaatttgcctgcgcgcctgtgctgggtgcgctatcggatcgtttcggccggcggccggtcttgctcgtctcgctgtccggcgccgctatcgactacgccatcatggcgacggcgcctttcctttgggttctctatatcgggcgcatcgtggccggcatcaccggggcgactggtgcggtagccggcgcctatattgccgatatcacagatggggatgagcgcgcgcggtacttcggcttcatgagcgcctgtttcgggttcgggatggtcgcgggacctgtgctcggtgggctgatgagcagtttctccccccatgctccgttcttcgccgcagcagccttgaatggcctcaatttcctgatgggcattttccttttgccggagtcgcacaaaggcgaacgtcgaccattacgccgggaggctctcaacccgctcgcttcgttccggtgggtccggggcatgaccgtcatcgccgccctgatggctgtcttcttcatcatgcaactcgtcggacaggtgccggccacgctttgggtcatcttcggcgaggatcgctttcattgggacacgagcttgatcggcatttcgcttgccgcatttggtattctacattcactcgcccaggcaatgatcaccggccctgtaaccaccaggctcggcgaaaggcgggcactcatgctcggaatgattgccgacggcgcaggctacatcctgcttgccttggcgacaaggggatggatggcgttcccgattatggtcctgcttgcttcgggtggcatcggaatgccggcgctgcaagcagtgttgtccaggcaggtagatgaggaacgtcaggggcagcttcaaggatctcttgcggcgctcaccagcctgacctcgatcgtcgggcccctcctcttcacggcgatctatgcggcctctataacaacgtggaacgggtgggcatggattgcaggtgccgccctctacttgctctgcctgccggcgctgcgtcgcgggttttggagcggcgtagggcaacgagccgatcgctgatcgtgggaacgataggcctatgccatgcgggtcaaggcgacttccggctacgacgatcgatatgaaagcccgcggcggcaaacgactcgaacgggtgccagaggctatcatggatgacgttcttggtcgtctggccacgattctgacttgacccattgtttcgacagagaaccatcatgatgaaatcaggaaatttgataaggtatttgtggttttttatctgcgttgtcacatgcctggtgatggcgtatttatgcagcatggttaatccatatgatgacagaactttgtcctccgcctttaccagctttctgaggccaatcggggccgggtttggtacggcgattgtccttatcctgatcactagcattgcgcgagagctgcggtgtttctgcaataaataaaagcgtgccggcccgatatttatgccggcattgcctgtttcctgaagtctgcaatatactgtatataaaaacagttattggaggctagattatggcgctggatctcgtttatccccggccttcagtggaaggcgccgcacttcctttttttgccgaacccgttgcggccggctttcccagtccggccgcaggctatgaggcaaatgaacttaacctgcatgattactgcgtgcggcgtccggccgcaacgtacttcttgcgtgtgtccggagattctatgcgtgatgcccgcattcatgatggcgatgtgctggtcgtcgatcgggcagaagagccccggcacggcagcatcgtgattgccagcatcgataatgaatttacggtcaagcagctgcagctccgcccttacccctgcctgatgccgctgaatccggcttttccgccaatcaggtttgatccggaggcgctgcagatatggggtgtggttacgttcactgttatgagtcaccgcccgtgtacgccttaatcgacgtcaattcgatgtactgcagctgcgaagaggcgttccgccctgatttgcgggggcgccctgtggtcgtcctcagcaacaacgacggggcgcttgttgcggtcaatcgcgcggcgaaagcgctgggagtgcgccgcggggaaccctatttccgctgccgtcgcctgctggaacagcataacgtcgccgtattcagttccaactacaccctttacgcctcattctccgccagatttgcaaccgtagtggaaagtctggcgccgcggacgtttgtttactcaattgatgagctctttgcggatgcgtctcacatggagggcgtgatgtcgccgcaggcgtttggccagcttttgcgggctgaagtgctgcgccagacaacgctcacctgtggagtgggcgtcgcgccaacgaagacgctggccaagctgtgcaaccacgccgcaaagacgtggcctgccaccgggggggttgtggcgctcagtgatcctgtgagactgaaaaggctgatgagcctggtgtcggccggcgatgtgtggggggttggccatcgtaccgaaaaagggctggcgacaatggggatcaaaacggcgctggcgctcgcagagatggatatccgacttgcacgccggctgtacggtgtgacgctggaacggaccatccgtgagctgcgtggcgaggcctgctttgccctggaagaaggtaccggcgcgaaacagcagcttgttgtatcccgctcgtttggatcgcgggtcacgcaactggcgcagatgcagcaggcggtgactaaatatgccacacgggcaggggaaaagttgcgccaggaaaaccgtctggcgaaggtggtaacggtctttatccgcagcagtgcttatgacgccggcggtgtgccctacagtaaccaggctacggaagtgctttccgtgccaagccaggatacccgcgacatcatcgccgcggcgcagctggggctgtcgcgtatctggcgtgagggtgtgcggtatgcaaaagccggcgtgatgctcagcgatctgcagggacatgaaacccagctggatctgtttgcgccggctgcagtgcggccgggcagcgaaaaactgatggccaccatcgacaaaataaacagggaaggaaggtcaagggtcttcttcgccggggaaggtattgatccggaatatgccatgcgccgtgaaatgttgtctccggcctacaccacgcagtggaaggatattcccgcggcgtttatgcgataacagcgatgtgcaggccagtctgtcagccctgcagccgttcctgctcctgcagcaatgccagatggtatgcggcaatggcgttcaccaccccggccgtcatccgtgcatcggcgattgccgagtgtgcctccccttcccaggccaatgctgcctgactggccgcgctggccagcgaaatggtgccatagcgattggtggccccaaaataccctgctgccagctccatcgcgcaatatactgtcagttctcccagccagtcggccggatcgccatgtgcggcggccgtctgtttcagaatgcgggtatcaaagggggcgttaaagataatcactggccggtccccgatggcgtgtcgcagctgcagcgccacatctggccaggatggtgcgccgctgagtgccggctcatcgataccatgcacggcggcggcctgcgcctcgatggcgaccgtcggcttcaggcgggtttcaaatatcacccggccggccgcatcggtcaggccgatttccagcgcttctgcggtattacccagccccgtcgtttcggtatccagaaagagcggcgccagactcagccagtcgtgtgcccgccgtgccatccggccactggtgctgttaagccgggacaggacggaaagacgcaggccggcctgctgctgcttctcggtcggcggccgtttttcccgcatcggaacacagtctgcaatgcggtatacggcgtacttgccgccataggcgcttttgtaccatttcaccggctcagcaccgggagccggcttcattctgaattcatcgcgcaggcgccctttcgtgaagcaccgatcgccggctatcgcctctgcgcgtcgtgtggccagctcatcattcttcatggttattccatctccgtcaggcgtttgtgataaacctctctggcgatctccaggcctgcctctgtcagcccaaacgcgagcgtcagtgactgattatcccggtatttgctgagcaggccgtgacggttcagcgtatggcaggagcgacgaaaattggtatcggcaacgggctggaggcgtcccttattgaggatgctgaaaagctgcatcgccgggaacgggcccgccagctgacgttctgccagaccatacagaacaaacaatacgtctttttgcacaccggagagccgcatcgagggggctttactttgcatcttattcatgtgtcacgtcattaaggaattaatgacaaagtaacctaattgatgtgagacatcaataatattgtgttcataacgggaaatcaggagtccgaaaaagaaaatgccgctcttgacggcggcttgttttccctggtgtcgggagaattatggtttctggtctgggtcaatgcgcagggcgctgtctgtgagcaaatccagtgctgccgtcagatgctgactccgcccgccgtgataatccggccacggaagaaagggttttgacgtatcagacatgaacatgataccggtcgggcagcgccactccgtgggggttcccttgttgtaatgaaccagcagtactgaacggtcacactgcacgtcattactgtcatcgttgttcagtacaaaagtggccacgatgccggcatcgggcctggacacaaaaagcaggctgttggcaaaaataacggcaatcaccactgaggcaatggccgcaccggtataccagaaacggccgtttttccaggcccgatagccatacagaatgcacagcagaatggcggcagcgatacctgcccactgcgcaccgactgacaggcgggagagtgcatccatcgataacggccggctcagcgcataaaatgtgacaaggaaaggtgaagccagcgccagcgtcagcagaacgaaaacgaggggtaagaagcggttgccggtatagctcccattgccaagcgccgtcatcgtgcggatcatctcactgaacgccatgatcatcataaagcagcataaagccatgtataacattaaaacatccccaaactgtccgcgtgatatggccggattttcggcctgttccacatagtcccgaccccttatcggggtgcatagccctcttcagccagtgctttcatgatctggttgattccatccatcatcagatatgcctcaatatctgacgtcgccaggccgcggcagtcactgaatcccagccgcttaaccagttgcgccagagcctgcgcttctctgtgtgtcagctgtatattcacttcaacgggtttttcgtggcgaatttcatctttcatggtcagagtccttttttgatgtttcgtgaaatgtaccaggcttcttttgcctgagctacagcccagatccagacgagagttgacacaatgcacagccccgtcatgttccagaaatgacctgtcgtatattcatggccatgaagcaggaggccggcgccggccgccaggctcatgaccaggaaaaggacggctgcgccggcgtgataaatgatgctgaacatgtaaaacccccttctggcgacggtagcgccagtctctgtctgtattaaatggtcgccgcctgccgggcgtctgcctgcatcatggcgtcaatcagggcgccggcgcaggcaatattcaggcacgtcatgttcgatttcagggtcgccatcatgtccgcgcggcttcttttcagaaacggctgcagcggaccacgatagccctgtgaggtcacgataatcagctgagtggccgcatcatgcagtgccgacacgccggccagaatatcctgctcagccgcgctgatatacacgggtgaaccggcgatcacaaagcgatcgtccgggcagcgttgcatcagctcagccagggaggcgcagcggcccgccagcgggggccgtgccgtcagtgagccccagacggtcgccggcgcgaagggcatggcgtgaaacgttgcctcatacgcgaccgccggatcggtatggtggcgcagccccagaccggccgacatggcccacagctcgatgcccggccaccgcgccgcgataccgcgggcacgcagccagtgtgcgccccggtaaagctctcccgtttccagcaccctgccaggacggcgggcagcctgggcgatcagcctgtaccagccgtcaaaggcgtcatcaagggaagcgtcgcccggaaaaacggtatcctccacgctgcccgccttgcgccgggtacaggtggtgatgaggtgaagacgtttgttcattcagtgcctcccggctctggtgcgtctgtgcctgccgcgcgcggttcgccgggccgccgggcacgaatatcgggcagaccccggcaggcatcgttccagtgcgtgccccgatcggggttattggccagcttactgatccaggtttctgacacattccagcggcgggccagctccttctgcttccagcctttcgccctgagcagcgcccgccactcctgcggggaaagcggcgccgccgtgatgccggccgcatgacaggtcgttttcatcgcgtatgctcccggccctaatcctgccccggccgtgccggctggatttgacgcagtattgttgttgcctgccatcccgtaaacccgaaggtatcggccagttcgccgggcagtgacgcggccaggcgcggcagttcgtccggggattcattgtgaaagacaaaactgaccccctcatccctgatgcgcgacatcaacattgcaataagccggtgcggtccctcatggctggcgtattccgtgaccttttcagtaaaccgccggggattgctgcagctgataccccggtcggccaggaacgccagaaagcgctgcaggaaaccggggagatccctggcggtcagggcgggtttcagcaccgcttctgcctgggcctgtgtgaagccaaacgccgccgccagctcaggggaaaagtccggcgtgtcagcgctggcctcaaaaatgacgccatcgtttatggcctgctgcatgtccgggggcatattcccgatcatattttcacgcacgccccatgcacggccggcgctttcgttcagtctctgcggatcgctgcagctgacgccccgctccgccaggaacgtcaggaaacgctgcaggacggcggcgccgatctcgccggagacgggcgcttctccggagggatagaagaccctgacgttggtgttatccagtaaggcctgttcagcgccggcgtcggacccggtgctttttccgctccgccagaccgtattatttcgactgacgatggccggatcgacatgttcgctgtccacgatgctgttaatgaacgtcactgactgttcttcgctgaaatcaaagtcgtgctgtaacagggacagcagcctgtcatgccagtcatctttcacgcgattggcggcattaaccatgaacgtgacgttcagtacaccggccagtaaacaggcagcctggtcggccacatacaggctgaagtattccagcaccgcctgttcaaatgcagcgggatcgcagcagaccacttctgcagatcccagcgactgcagaaaccgccgggtgttttcggtcacagcgtcggcgggattgagggcgatggcgcggccgcgctgtgactggatgtcaaatgtgacgatacgctccgtgacgggcatccggccgtgatcggcgatgaagcggtaaagctgctccactgcttccgtggcggtcatgcccattgcctccagacgcagttcggcgcgtttcttcagctgctcatcgagacgcacggtcagctgggcgtttttggcccgctttttacgttcaaaaaaaccggtcatgctccttcctttttgtacagtcatttggctttacataatgtacagccatttgtaccgcacttttgcgcgtgggaccacattttttcaccgttgcggtttagcggtattctccgcagtgccgccacttatccccagtttctgtggataacgctatatgtagtggctccgtcagctggaaacggcacaagatatggtgtccgcgccggcagccgtgcggccgttcccctgccttcccttccctgttgatttcgctttttccggtatttacgttttttccgcaatcacggtgccggcgaaaaggtattttccggtggttgcgggcgcaaaagtcacagggtgaaaagataagggcacgaagcattccgcaatataaactgcgtgacagtcacgccgggtgatgactacccgcgggcgattctgccaaattagctattagtgctaaatctaaatatttataatttacgcatgaaacctaatttattagagtgtggttattcagtaaacaacagcaaacggagtgaacgggatggaatatgaagcgtacaaggttcgcggttacggcttcggactgaaaaaagagatcaccggcgtgctggggcgttaccccaccctggaagaggcgcagaaggtcgcggatgactacacgcaggcctgcctgccggataacctgccggacagctacgatatgggcatggtccggccggtcgctgccggcgaacctgccgccctcgatccggacgtgtttcgcggtccgggcgcgccgttcctgatggatgccggccagataaccacgggtcacacggtatgcctgggcaaagcagcggatggaaaacggttttattttggcggggagcattccgatggccaccaaacatataaatgatgagctctggaatcgcattgaggcgctgaccgtcaaagctaacgccaggcatgggttgttgcggccgattaaagaggctgaagtactgcatcttgtcctgcagcgtggcctggaactgcttacggacgacgatctgctgcagctgggcaagtaccggcgtccgataggatttgtgctgcgtcgtccgggcgtggaaatggtgaagctggatatgctgaatatggcagatgcggcaaccgttcttctgcgcagtggacccgcaacgctgtgcatctggagccgcgacgatatcctgcgcgaggccagtgaagcggtgatccgcgaacggctgccggatgcagccctgctgtcggagggggatgaccgggcccggttccagacgctgctgcccggattctggaacgccgcgcaccggggcgaaacggccgtgatttccctgcgtgctgacaatgcggatctcgctattgcacgcatcactgacctgatgtgcgaatccctgctgggttataaggggcagcgggcataccgggcgggtgaaaacgaacagggagaagagtcatgaacatgcgaggcattctttttgtcctgccgctgtgcgccgggctcctggcgggctgtgataacaaacaggaagcgctgcagtttgaagccgcccgcacggcggcgctgaaggccgacgatccgtttctggcgctggactcgttcgaacagcagtccggcgactacggccgggactgcgactcgctgggggcatttctgtttggtaattttgcttccacggataaagcatgtcgggcgcgggatgaggccacgcgggatatggtcctgaaagccgtagaaaagggttccgtgccggcactggtgttcctgtttgacccaggcagcaagacgccggatgtgtatccgcgcgatattgacccacagaaggccgggcaggcggctgagcggcttgttgcactggcagagtcggcgccggtggacagcaaaaatcaggcgctgctgatgcgtgccggcgatgtactgcaggcgggccagtacgttctgcaggattcccggagagcggcggccctgtacgtcaaagcctggcaggcaggggatcagcgtgccgcggacgagctggtcaatgtctatcactttctcaaggacagccggcgtacctacttctgggcggtccggagcaaagacgtgcagcagctggacaatcgggctgagctgaccggagatgagatcattgctattcagaaactggctgcggacagttcacggaccgacatttgatgaaatgaataaggatatacaaaaggcgtccaactggtgaggaagggcgccttttaattactagacatttttactacttaactttctgtcggagtaaacggaaaattaatctgttaatcactgcgagtataaatccccgctaagcgtggtgcaactcagtatcagaaatgaccgccggtggttgaatccagtgccctgaacaggctttcctgggctgcttccagcagacgacctggcagacggagttcatttacgcggtaactctgcccggtctgtgtggtcacggtgagataatactgatcgtcaaactgctgaccagcaccgatatgcacgatggtggccgtgtcgatccagcagaggtttgtgccttccgcgtcctcaagtggtttgattcggcctttcagccacttctgcgggcgacccggccggcatttgatcgtgatgccgtgcttatagccgcgggttctggccacaaaggccgctattttatctttatccagttcctgcgccggggtgagcttactgaaactggacggagagaagttatactgcagttcttcaaacaggttcaggtacgtggcttcatcatccaggttgtccccgcgttctttcagcatggagccccagatcctctccatttcgcccctcagcgccagtttaaccgcctcaaattcttcctgatgttcatcgtagtagttgatatccatcgcgtctcctgttgtgtttatggcgaatttgcggaattaatatacataaaaaatgttcaaatatgaacattttcaggctataaatacaggcagattataacaggccagtccaccaggattcggccaggagctgagtacgcatgataaagaaaccacttattgccggcatcatgctggctgtggcgcacaccgccggcgctgcgcagggtgatgataccgatctgcagcagatttataccgaggcgggcattcaggggatatgcagcgccgggatgctggctgtgccggaaggggccagggatgaagctgtcccgcagaacgtcagacacctcctggttcagtgtgcctatttcgacgagcagcagttgcgccggcgtgccggcctgccggctcagacatttagccactggcgtgaccgcaatgcggtccttaatctgtcgtctgaccaggcatctgatgcgctctggctgtcagcgtggcgtgatttccgggcacaactggcctcggcctacccggccccggcggaaaaaaaggcacaggggaaagaggccgtacccgccggcacgaagcccggcactgagcatgggagtgcggcggattgaccggttttgacttgatcctgtggcgacggggcctgaactggacccaggagcgcgccgcggctgagctcggtatcagccgtaccagcctggtaaaatatgaggacggtgagacagtaccccggaccatacaacttgccaccgtcgcgctgacgctgaaagcggaatggcccaccctgaaaaccatgagcaaagacaggctgctgcggcagctgaagtatgaagtgctgaggatcgataatgaatgagtttatccatgttgtcggctcatttatccttgcgggtatcctgctgtatggcctctggcagggtacacggcgacgccggcgccggcatgaacggaaacaggccagtgcggtgcgtgtgatcgataaaattaatacgttcccgcatttcgggcagaaaattgcctatctgcgcaagattgacccgtttgtgttcgaagagctgctgcttgaaggctttgaacgccggggctttgaagtgatccgtaaccgccggtataccggtgacggcgggattgatggccgcgtgaagattgatggccagacctggcttatccaggccaaacgctataccagctatattgccgttggccacgtccgtgacttcagcgacctgctgaacgccaccggtgcccgcggttttttctgccataccggcaaaacgcgcgaaggcaccaaatcgctcattcgcggagacagccgcatgatcctggtcagtggccagaagctgctagaccttattgccaccgatgcgccgtttatcccgtatccgggataccggccaccggccgaaatggttcatcctgaacaggagacaacatgaaactgaaaggatacgtcgccgccctgccggcactgctgctgacagggtgcgccatgctccccggacagcccaccgattatgaccgcttctgtaacgtctcaggcatcgccagccacggcgaaacctaccgtgtcagtgacagtcaggatttctggttaaccccgaacggccgctatctcagccaggctgagtacagcagtccggcagatacgcttcagaaactgacgggcgtggtcagtggtgaagatcctgaccaggtgcggaagaacgccgtcagggtaagggtgttccgtgttgaaagtgagagcgccagtaaaggggcgtgcctgcccgtgcgttatgacgataaaaacgcgcggcggaaaatggattcgctgactaacggccgccgtatgatggtgtacagtgaagatgaaggccagtctggccagcagatttacaataacagccgcggtgccggcttcagttacaggttgctatgagtggattgaaaggccgccgtatcagtgcggatatgtttgccataatgctgaagatatctgccgggcagctggtttttgcagcacggcatgacaagccgttacgcggactgaccctacccccttacagaatcgaaggcggaaaaagctggagctcgaccacctccacccgcagtatgacgtttgatatgcaggatgcggtcgatttcagcgagaaatttcataaaattcagtcgcctgacggctgattagcggattttaaaaaattgaacactttccccctgcaaatttctcagaattcacgtagcgacgccatcgggacatatgcctacttcgattatgccgatgcgctggcgctgcgggagatggccagccaggctgatttacccaaatacctgctggccccggaggtgtcggtgtttctgcggtatgtgccagacctgcgccagcaggtccttttcgataccctctggaacacgggcgcgcgtattaatgaggcgctggcgctgaccggcgccagctttcagcttgacggttcccggccgtttgtccggctgaagacgcttaaacagcgtcagcgtggccgcggcaggccgggcaaggatgaagaggtgttccgtcttgttcctctgaccgatcctcaatatgtcaggaaggtgcgtgaatttctgaccaccctgcgtattggtaaacagcagctgctctggccggtgcagagtgataacacgccgcgtaactggatcaggaaggcactggacctcgcaaaacgtgactctgtcacttttagtatccctgttacctgccacacgttccggcacagcttctgtatgcacctgatccagcatggggttccgctgaaagttgtacaggcttatgccgggcattcccggctggaaaccacagaaacctacacgcgcgtgtttgcgctcgatgttggtcgtcagtatggcgtccggttcagcctgccagacagtcatttaacgctcccggaataatctgtatccctccctgatatttccatataaatctaaataaatccattttctttctaggtcagaccgttttcagggcggcctggccgtcttttgttggtttttattctatatttttctgtatttttctatattggtgcgtctttttgtctttacacgtcagcagggatgcgtaataatccatttgaaagctgtatttttcctgatttctacggtttttttctaaataatgtgaggttcaaaatgcgtatttttgttgacgacggttccaccaacatcaaactggcatggatggaagacggtgcggtcaaaacgttgatcagtccgaacagcttcaagcccgaatggtccatgacgttgctgacagattcatcagtgccggcttccgccaactatgaaattgacggggaaaagtacagctttgaccagctgagcccggatgccgtgcgcaccacggaaacccgctatcagtacagtgatgtgaatgtcgtcgctatccaccatgccctgatgcagtccggcattgaaccgcagccggttgatgttgtggtgacgctgccgctgggcgagtacctggatgaaaacgatcagcctaatatggccaacatcgagcggaaaaaagcgaacgtgaagcgcgcggtggccgttcaggggcgtgagagctttaccgtgcgtaaagtcagcgtactgccagagtcggtccctgcaggcttcagtgtgctgaaggatctggacgatctggactccctgctgattgtggatatcggtggaaccacgctggatatcgcgcacgttcgcagcaaaatgtccggcttcaccaagacacattgcgatcctaaaaccggcgtatccatcatcaccgaagccgtaaaacatgcactggataccagcgtgagcacccgtaccagttcgtattttgccgaccgtctgatccaggcgcgaaatgacgaaacgtttctttcccgttacctccagaacgctgaccagcgggcccaggtgatgaaggtactgcatgagcgtgaaaaagccctgacgcaccgtgtcaccgattcagttggccgctttgccggcttcacgcacgtcatggtggtcggtggcggtgccagcctggtcgccggcgcagtgaagcaggcaacgggggtgagtgacgatcgtttcttcgtcagtgataacccgcaattcgatctggttctgggtatggtggcaatgaagggctgagtatgactgataaccgtaagtgctcgttttatatctacccggagcgcaatgcggcggaccgggtagccgacggctttcttgagaaactgcctcagaaagagcgtggccgcgcgatgcgcgccatgatgctctgtggtgccgcgttgatgaagcaggacgaacgcctgccgttcctgatcgctgaattcctgacggagagcacctcgatgcaggatattcagcggatcatcagcagtacgctgccgcagcaggatacaggagaaatggcgcgtctggttgaggcgtttctgcaggccaccggcggtggcagcaatacggtgtcgccggcaggcggcgctgcgccggaaaacagtcccgccgtgactcccgaaccggccgcaccggaggataagaatctggaagaaacccgtaagaacgtccagcatctgttcccggatgatgattaagggggcagcgtgaaaaaatatgtgctgttaaccctgaccctcatcgccggcgccgtggcagcaaacccggtaacagcggccggcaatgttcaggcgcaaactgcgactgaagtgaaaaccgggttttcacccggcgggaccgcattaaatctggttctggattcaatcgaccacgcgcagcatgagattctggttgccgcgtacagcctgaccagtcgtcctgttgccggcgcgttgcgtgccgcaaaaaaacggggcgtatcggtggtggtggtggccgatcgcaaagcgaacgacaaccggtacaccgaagtgaactttctggccaataaccgcgtggcggtgcggatcaacgataactacgaggccatgcatgataaattcatggtctttgaccgccgcgccgtcttgaccggatcgtttaacttcagcgccagtgcagacagtcgtaatgcggaaaatgtggtgctgatcagcggcgcgcctgcagtaactgaagcctacgtgaacgagttttcccgtttatggggcgaaggaaaggacgtggccccacggtactgacctacagcagcggcagaaactctgccgcctgatttttaacttgatttatatggctcattcgtcgtattatcaaggctcatttttcggtagcgccccgactggcgaaggagtaaaaccatgcaatcattccccctgttcgtttcccgtgtcgtacctgtgatcaccctgagccattgccttcggcagcggcgctggtccctgcacaacacgtcatgccagattgacctgagatcaggttgtcacctgcgctcacagcctttcggcacgctatccagacgggaaataagcgaccgaccaccggctatgccccgcggttactggttcaggcaatgcagccgggatgtcatgtttacggggcgcatcgatcaggcagatcctatgaaacaggtctattacaacgaaggctggagcggtccgaacaagtatacgtttgaagtttatcagctggagaacgggagttaccgggcgctggcccgcaaatggaatggcaaaatcaacaaggtgcagcaggaaacgcagtacctgtctgacacgcgggaggggctgaagcaccaggactatccccgcacgcgccaggtgaaaatttttctgaattcagacttctgggagaaaggtaatgattaagatggttagcgtcgttccgcagccggaaacagtgaagacgctccgggaaaaaatgggcatgactgaaaccgcattaggggctgtcatggggtacgaactgcgcgcctggcagcgtaaggaagctatcagcgacgatctcagccagtacaacaagaccagtcttcgcccaggcgaatataacatgctgatgctgatcgccggcgtgcatcctgattaccgcctgaacagggcattcagtccggatgatatggtcaaagatcccgccacggccgaagacgttcgccgcctgcgtctggcgctggggctgaagcacgctgaaattgcagcgctgtttggctataaaccggcgtcatggcagaccaaagaaaaggctgctcagcgcggcgtaaagctcaaaaccggggaattcaatttcctgttacttctggccggtgagcatccttcactgcagctggttgagaaggtgaaataaggcccgcggcgaccagagaggaccctttatgatcaccacacgccgaatgttaatgctgtatgcctgccttgtgcccgtagccctgacagggtgtgatctggctgattatttcccgaatgtgccacagcccgtgcccgtgatttcgcccggctatcaggttgatatcgatggccacccggtccgaatatcaggatttgatgactgcccgcgggataaagatgaagcccggcacgcagctgcacaagatgagaaaggctgcattgtgctgtctgcagagcgttccgaggtgaaggtcagactgtatcagccagccggcaccgtcaccgaaacctggcggatcatccgggaaaaagacgctcgccagaacgatattattcgcatgcagcgcccgaacggtgcgcttgtgatgcaaaatttacaggggaaagagtctccttaaaccaggaaaataacaaaattaacttgatttatatggctcattagacgtataataattacatcgaaaggcaatccggtctttcgacggtagcgccccgaccggcgcaggagtcaccttatgactgctctcaattctcagcaacaggctgatgttgcccgctatatccgggaacatggcaataaaaccgttatttcccctgaatggtcacttatttatttctcgctgtgtcagtgtgaattacatcaatcgcatgaagccattatttttcataatggccgacctgcagggctatttcttctgaatgaaacaggcgagctcaccgaattatatctgctggggtcgctttctgacattctcaccgccgctgcggtcaacaaatagctttcagggagtaaatacactatgaccatgcaatcagaacttgtttttactgatcctatgctcaacgtcgttattgctgaagttaagcgttttaattgcccgcttctttttgtcaaagatcatggcgtgtatgtgatggcggcgaaaggtgagaaaaacagtaatgggatgcacaatgtctgttacgccaatggtttcaatccggatacaacggattttgacgagctctgggaccggatgcgtgatgcctgtggcggggatgatttttgcgaatccctggacctcgatccgaggtcgattgagcttctcagccgtaccaaaccctgcctgaaaattatgctttcagagacggaactggaagttattgcagggggacagaaataaaccataaaagcccgttaaatcgggcttttttatatttaactgtatttacatataggacaaattgtcctaatatatatttgtggccgggcatggtgctcaggtcccctaacgagggaaatatatgaaattttcagcagaagaactcaacattatccgccaggcggacgccattattgccggtaaaattaccacaacggattttatcaccggcgccgatgccgcgcgttccttactgcggtttcgtcttgttgcagaaccacgcgaatcattctgcgcgttctttcttaactgccacaacggcgtgattgcgtttgaagaactgtttaagggcactatcagcagcgtcaccgttcatccgcgtatcgtcctgcaacgcgccctgcatcataatgccgccgccgtggtactggcgcataaccatccttccgctaacactgcacccagccaggctgacaagctgatcaccgagcgtctggcgcaggtgctggggatggttgatgtcaaagtcctcgatcacctgattgtgggcggcagcgatatttactcttttgctgaacatggtctgatttgacaactgcggccgcacagtggtgcggccggctctggaggcttgctatgggcaataaatataccgtgccgcttaacgtgggcgcgatccgcgaaagttgtttccgtcccgctgataaatggaccccgccgacgggggatgaactccgctatctgctggaaaatgtgctcaacctcagtcaggaggggctggcgcgacatgttggcgtgaacggcagaacggtgcgccgctgggtgaacggagaaagtgacatcgcgtattccgtctggtgtgtcctttgcatcgatgccggcttaccgccgatctggaaatagccgcgcaaaacaccgacgcgcaaacgtgcttcctgaacgtatcggatatacgaaggaaatattatgaatactgataactattttttcagagtcccggccacacgcggaatacagggcggcatagaacaatacatgctgacggttcctatggtcgtgttacgtcgtattcttgcaatggacaatgatggcgatgttatggatcgcagccagcgagaagcgaacaaaacgcgggccaaaaaaatacggaattatgttgccggcgcaacatcaaaacgagctccctatattttgccgtcaattaccggaaatattgatagtcatgttgaattccttcccagcgaactctcgccggcagtcggtattcttaaaattccgatggacgctgatttaaaattattcgatggccagcaccgtgcgctgggtatttttgaatttgtacgcgattattccaacacggaagatacgatcagcttattgcttaccgttggcctgccacttgagttacgccagcagtttttcgctgatattaataacaacgcgagcaaaccggcagcggcgatcagtatggcatataacaacaatgatccggtcaaccagctggcaatgcatctggcccgaaccgtcaccgggctggcgggcacagtggattttgaacacaacgtggtcccggcaaaaagctcacggcttatcagctttaaagcgctgaacgatgcgacgaaaaaaatgctcaacctgcgggccaacagcattccctcgccgcaacagcgtgacatggcagaaagactctggactgcctgggcgcaggcgatgcgctggaatgatattgcccaggatgatatcgccgcggagtaccgtcaggaggcgctgggactgcatggcatcatgattaatgcgatcggtatggcaacggccaggatgttacggcacagaacccctgaaagcatcgagaacctactggcctgtgctgaaaacggtgacaacggttttcattaccgggagtcgtttgtacctgagtgctgggaagggaagtgtgttgatccggaaacaggcaccatcaaaaccgacaggcggtcgctcgaagcaacggctgaagcgttgcaaaagctgatagatccgttcgcagatgcgctatggttgcgtgattacctgccagctgaggaggcgactgatatggcattactgaaatatgcagccgacatcgagaattataagcagcgtactgccgcgccgatgatcaatatcgtggagaagctgaaggcgctgggtgacggcgaaccgcagttcagagcgtcggtgctggcttccagtgaaggactgagccactatctggccggggctgaaggctgaccaggtgtccgccgcctgcagggcaatacgggcatcgaacccgtattgccctgaaacgcgctgagcagctctctgagcgcttgtttcaaaaacctgtaccccactcgcctctattgatttgacgtgtccagccggccattctggccttatctgatgttattccgacatggcatctgccagttaacctacttctgacatccgtaccgtgtgtcttccctgttcatttctgctcagttgccggcgttttctggcaggcctgtcgtactaaaacacctctgggccccttcggctcctgcccgtcgggcagtcgtcagccacactccgctagccaggcaaagcctggcagctccgccgaaccccctgcggggtttcgggctatcgctagtgtctgatgcctgaacccctgcaccatgccggtaaagtgctgccagctggctgatggggatctgaaagcaaaatttacagggaaaggagtccgggaaaaaacaaaggaaattaatcagataacgcttgattgttatggctcattagacgtataatgatcacatcgaaaggcaatgaggcttttcgcggtagcgccccgactggcgcaggagttacaaattatgagtaaagcactgaccccttcccagcttaaagctgaactggacacgcagaaagatgcgctgctgaaggcgtgcctgtatgccctcaacgaaatccctaaccgccgtttaagtggtccgtattcgtccacgtatgcgctggccgcgcagattagtaaactgcttcagagctgcaaataatcattgatgggctggcattgccagccctgttcaacgctaacggtagcgccccgcatggcgcaggagtctgaaatgtcatctaaaaatactctcccgtcggtcgatctgatgtcctgccagcatggcggtgacgtgatcccgtccattgctattgagcgcattatctcgttgcgggatgaaggtatccgcgtctatctcgacggcatagacaagctgcgaaaagcgcgcgatctgatgcagcaggccggaggctgtcagtacctgtatgactacgacaaagcagtagagagcgccatcaaatggagcgacaccacgccagaccaggccgaaacggctatttcccgcgttatcgacggaaaaatctgggatcggctgatgacggaaaccggcatgtatacgctgatgagcaacaagcaacgtcaggaatgggatcggcaggtggcaggcaaagaaatgccgccgataacgctggataacgttatgagcacgttccggcacctgaacgcaagcaaggcggatactttcacgcaagggctgatagatatctttaaatccctgtcgtgggattataaaaccaataacccctgcatgtttggcaagcgtatcatcatcgcgccgttgctcgatgtctggcgcagtggctgggtgcgcttcagctcagacgggcacaccaaaattgacgatctggcgcgtccgttctacgtgctggacgggcgtaacgtgcctgattaccgtgtttctgacggcgccaaactggatgcttttttcagtgaaaaccagttcaacggtaaggtgttcgagtgtgattacttcagcgtgcgatattacaaaaagggcagcgcgcacattacgtttaaacgcccggatctggttgagaagatcaataatctggtcgccagtcactatcctgggatgttaccccctcgcgtgtaatccagacggcaggccgcatttgcggcctgaaatataaacagggaaaggaagtattccccggcctgagaaaatcatgccgggcatgtatttattgcttgattagtgtggctcattagacgtataatgatcacatcgaaaggcaatgatgcttttcgcggtagcgccccgactggcgcaggagtaataaacatggctattcaatcccttcagttccgtaacggctccgtttctcttggtgatgtttttgtttccagctggggttatgagcagacaaatacctgtttctatcaggttatcgccctgcgcggtaagaaaaccgccgtactgcgccgcatcgctgcgcagcaggtgaaagcagacagtgcgatgtcaggcgagttaaagccggttttgaacgagtttaaaggcgaacccgtgacccgtcgcattcgtgaaaaccatgagcatccctctgtcagcattgacgagtacgaacaggcttataagaccgatcctaacgaaagccatttttacagcacctggggctgaccgcctgctacgccccgcaaggggcgtatagtcaaagacttattcaaccaaaacgtgaatactatgcgaaataacaaggcgctcaggacgcagatacatggcggaaacagtttatcaggcgcggaggcgtttttaccttctccgttttcggcgcagccgtaacccggacacactggaaaaaatgtacgaatccatgcgggatcgtggacaggtgccgccggaggactccgaggcttttgaggctgccgccgaccatcgccgcgcagagctggcatcgggacgcatctgggataagatcccgccgcatgtctggcagtatgtaaaataatcacagcgcccccacgaggggcgtttttttgcttgtatgtatggctcattagacgtataatgtttttactgattcaaatcagtaccttcctaaacagccttacaggtgcattatgacctcttctgaaatcctccataacctccctcagattatcacggccgtggccgccctcattacagccattaccgggctgattaaagtcatgaaggataagggtaaggactgaaccgccgcagggggcgacagccccctgcatcgctaaccggcagcgccccgattggcgcaggagtaaaaaaagtgacaacgctgcatattcgagaaattcccactgatggcgcaccgcctatctgtgcaggccgctcaatctcagatgatgacctttgcgcctcatgccagcatctcgactatcgccctgggcacctcagcgcctgcctgctgtcaggttgcgggcgtgacgactggccttcagaactggatgaaaacggctacagcgttgcctgtagccgctatgagcgtgaaaaattgcttgattaatatggctcattagacgtataataatctcatcgaaaggcatccggtctttcgcggcagcgccccgactggcgcaggagttttacaatgcacggtaacgtaaatgaaatttgcgcaaggcttctcgatagttttgaaccacagcagcggatcagccttctgatctggactgctgaagatgtccatgactgcaccagtgatatgaacctcactgacgatgaggccgaagccgttctggcagaaatcgcggaatgcagttcacacagccgttatggcgtcggaaaagataccgtctggagcctggcaaagcaggtgcgcgaagatgccgcccgcgatcgaaaaatcgaggtcaatgccgaggcgctgcaaaaagtcgtggcgctggcggcgcagtttatccggctggaagagatccagtctggtgaaggagctgcacgccgactttatccgcaggagtctgaggcgctggagtgcatcacgaaggtgattaacggctaattcacgcagtaacgcagcgaactttcacggcagcgccccgactggcgcaggaggaaaaaatatgtcccagaatgaagcctattacgacacgctggccctgattgaaaaggggcaatggtgtttcggcagtatggacgaagaaaacgaagagcgggcaatcaaggccgctaaggcgctggcgctgtttgcgcgcctgaatgagcaggactgcgacggacacccggtttccgaaatcatcgttgattttatcgctaacctgatgcacctgggggaagcgataaattttcgtgtgctcgatgaggaaagcgcggtgatcccgttggtcaggatggcagccgtacactttaacgctgaaacaaagtcatagtaatcatgtgaatcagaggggggcaggcgtgatcctgcccccaaaaatggacaggggaaagagttgtcccgcctgaaaatattcaggcaggcggggatattttgcttgattgatatggctcattagacgtataataaacacatcgaaaggcaatccggtctttcgtcggtagcgccccgactggcgcaggagtacagaaaatgtctactgaactgaatcaacccgccatcactgcacgcattatccccaataaccgccgcatggcctttctgccacgcctgttcggcgcctggtatctcacaggtgaggccggtgtctacaaccacgcacgctctctctgcgccgattatcaaggcggttcctgggagttcgtcgaactgtcttgcggtagcggcttcatgtacccactgagcgcagagcgcttcacggtatcggttagcggtaacttgttcgaaggggaactgtccgcagaagctaccggtatcgtgctgaccctgttcaccctgaaccacatgatctggcacgcacatgaagaaggctatcagcacatctgcgacatgctgatcacccagcaggaaaagctcaaattatatgccgaccagcacgccgaagccggtttgatttaccgcgcgattgactgaaacggcggcccccgcgagggggccaatactgacagggggaaaaatgttaacactaactgatattcgcgccagcaatacggtgctggttacggaatttggcggcgtgcgtgctgtgcatttctgtctgcatgaaaaattaagcggtagcgacaacgatctgtggtttccgctggccaatggggccgacctgttcgaggcgctggaaagcattatgtgcatcaattttgccgctgctaacgttgtctcgctggagttcctgcggcagaacgggaagtgcaaagactaccggatcacctacaacaaggccaaattcaagccattatgttagatttctggtagtattaaatgttcatatttgaacaaatttaattaaaaaaatacgtctgatgagttgataaaatggctcattagacgtataattattacatcgaaaggcaatgaggcttttcgcggtagcgccccgactggcgaaggagttgaaatgaaagaagcagaccaaaaaaaagtgaatgccgtaatggcaattgctgattatctcggtgtaaaaaatcaaatcgaggtgatcgaatactcagccgaatcagtgcaggttgagtggcgaaatcctaaaacaaaacagcttattcatcgtgattacacttttgcaatttcctttgtaaaagactttgaaaaagccctgaaatcgaacgtgaagttttactgataaagagggggagaaatcccccttaatcctcccattttttactgataaacatcaaatatatgaggtgtattatgtggaaacgtggcgaaaacaaagtgattctgatggggcgtgctggcaaagatgcagaagtgcgttacaccccaaacggaacggccattgcgagcctgacgctggctactgaaatcagctacagcgacaacgaagggaaggagcaaaaagaaacggaatggcatgatattgtcatttttggaaagaaagctgaagcagctgggaagtacttcaaaaagggtatgatgctgtattttgtcgggcgcattcgtaataacaaatggcaagggacagacggcaaaatgcgcagcaataaggaaattgtgatcgataataacggcgaaatgcagatgctgccgggagctgggccacgtaacaccagtgctgaaggtctgggaggtactgaaaatcatgatgaaccgccgttcccggatatgaatgattacccgcagtgagtatttgagtgagaacggcccgcaggacgtgggccgccaacccagaaggggggaagtatgaatattagtgaacaatatcgggtggctgtcgtcagtaccggccatgtaaaaaaagaagacgcggagctactggataaggtctgtaacgatgacttgtccggtgaaggtttattctggatcgagaaaaatcgctatgggtggatattgcgtatttctgctactgaacaatggcgtgtggaattaaccagggcggggatcagtgcaagtgctttggataatatagccgcgttaaatggtgccgggttcgactggatttggtttgaccgggatgcagaacaggtgccagaactgaccgtttgggagtggtaaaccggttaaaaatatgcctcgaaaaggggcatttttttcttgcctaaaatggctcattagacgtataataagcatgaagtttaaaagtggatattgattaaaacgctctgtaaataacgtggagagaatattatggcgaccttatacccttatgaggtttatacccttattgtgtcagaattaatcgtaagccattttaatgatgtcgctatggtatatccgggaagcgaagaaattaaaaagaactacgacagtgaattaagtgctgtacaggtagttaatggcatcagagaaaaactgattgaagatggtgttatacagtgcgaacagtgcccactgctatccgagtgggatgagaacaaggttatcaaccagctaattaaagattaccgcctgattctcaatcgtgactatgaactaaaccgtgttcgtcagcagctggccccggagctgatcgcacagatagaaaactggaaagagcagaagtattccgattctgcgaaggaatacgaggtgctctatgaaaccatcaacatcaacggaaaggaggttacaggaaaactaaacgtagttgcagtaagtcgctttgatgctgttttcatcgtaggacagcgtcacgctaacgataactttttggttctggatgtaaaggaggtgatgaatcattgaagtccagaaaaggtgccggattgcgccaacaatccggctgggtaggcaatttaaattgtgaggttcaaaatgccgttgtggattatacactatgctgaagatgtcgcaaacggagtgcaaacaaagtcgcatccgatgaacgcagcgcaatgggatcacttccgtttctggttggcagtgttccggcggattaaagaagccgcgatgtaatctgtaactgtctcaggggcgctgtatgcgcccctttttaatatccgtgcccggactcctttccctgtaaatttatcgggatcaagggcggatcgtggccctgaacgtgcgcaccattgagcgctgcatcgcagcggaaccggaaaattttttccggccaatgagtgcccttgtcaccggaacggtgacaagtatgcacgcactattacacgaacaggcagctcccatgcccggcctgacaggccgggagctatatcataatgtgggatgctatatgcgaaaggtgaaggttaatactgcggacctgaagcatcaggattctgacctgtagagccattttgcgtgcgctgctcttcgtcaactccagtggttttttttcggctgttgctggttcgttttttaccaactcgtccggcagtaatgtccttgatatcacgtaatgtaacatctgaaaaacccgcatcgttgaggaatttcagtatctctttatcttcataccctttcgaatctttcagcttcttaatgtcagacttaagttcatcgagtacgtgctttttctcaagcctttctgacgtcacatcaggcaagtcgtcgagaatgcttttgaattttccgatgtcctcactgctatagaatgctttcttttttgcaggcatatgccctcccaataatgtcaaaatcgaccatgcctaaccggagaaaatcggacaaggctattcaggtgattttacatgatgcccggcatgattcaattgctctgcctgccaggcccttcgctgctggcattcaaacagcgccgatggtagaattcaatgaggtatttaagtacgggacaagatgtgtttttggagtaccgccgacacgcggcggccgtccaaaaacgtcttggtcagggcaagccccgacaccccctaacgaggttagctatctgatgagccgtagcgagagcagaaaaacagacgatcgaatccaggtgagatgttccacagaagtgaaagctaaactcaccgaaaaggcccatgaagcagggcttagtcttagtcaatatcttatcaaatctgggcttggaaagcgcattcaatcgaaaggtaattacaatgcacttgccgctcttgtaaagataacagcccttcaaaaacatctgtttaacgaaggtgccggagttcatagcaaggagtattcagagattctgatcgaggtcaaaaaggccgcacagaaactgcaacaggagatggatggtgatacctaaaatcattgagggccgaagggacaaaaagtccagctttggtcagctgattaagtacatggcagacaagccgtctcaggagttgactgatacagttcagcctactcctgagacggctttagctgttaaatcggatatgttcgaagggttaaacaattacctgacccgtaagcagaaagtgatcagcacaccggtggatgttgagccaggtgtacagcgtgttgtggttggtgatgttacctgccagtacaataccttctcgctcgatggtgcagcacaggaaatgaattcagtttctcagcaaagcacacgctgtaaagatccagttatgcactatgttctttcatggcctgactatgaaaagccgaatgatgatcaggttttcgattcggtgaagtttacactggcatcgatgggcatgtccgatcaccagtatgttgctgccatccaccgggatacagataacctgcatgtgcatgttgctgttaaccgcatcaatccccagacatataaagcagcctcatcgagctttaccaaagacactcttcatcaggcgtgccgcttactggaattgaagaatggttggtcccattcaaacggtgcatatgtggttaatgaccgtcaacagattgtccggaaccctcacagcaagaaagagcggggcaactggcgctcactcgatcgcatcaacaagatggagaataaagagggggttgaaacgctctatcgctatatcgttggtgatgaacaggtaggcggtagtcgtcaaaacttgatccatgtttcagctgggcttcgtgaagccaaaagctgggatgatgtgcataagacatttgcagacatcggtcttcgtgtcgaaaaagcacaggggaaaaagggctacgtcattacccacgaacatcaaaaccagaagacggctgttaaggcgagtctggtatttaacaaggcgcaatataccctgaagtcgatggaagagcgctttggtgaataccagccttcacatattgaaccggccaaagtatcggtatttaaaaccgcatatacccctggtgcttatcgtcgagatgccaataaacgtctgcagcgtaaaattgagcgcgcagaagaaagaatgctgctcaaggggcgctatcgcgcctaccggaacaatttaccggtatattctccggataaggatcgcatcgccgatgaataccggaaaattgcccaacatacccggttggtgaaaaataacgtccggcattctgtttcagatcctcataccagaaagctgatgtacaacctggctgagttcaaacgcctgcaggcggttgccaatctgcggcttagcttgcgtgaagaacgcaacggtttcagagcggctaacccacggctttcttaccgggagtgggtcgaacaggaagcactgaagggtgataaggcagcgctcagccaaatgaggggctttgcctattcctccaggaagaaggagaagtataaacagcagctggttgagcagatcgggttcaaccgtacctttaacgccatcaccagtcatgatcgtgatgatgtggccgtaatggcctcagcacgtcatggtgtcaagccacggcttttgaaagacggtactgttatctttgagcgcgacggtaaaccggtagcggctgatcgtggtcatattgtgctgacagaaagtaatggaatcgacaaagagaaaacggctgatcttgcaattgcattaacgattgctggtaaagccaaatctgtacgtgttgatggtgacggcgagtttaaggaactgtgctgtaatcgtatcgtcgatgcagctgtgaaccacaatcaccctgtcgcgcagggcattaccttcacagatgccgctcagcaggcgtatgctcaaaacgagaaacatcgtttgattcgtgagcaaaataatagtaaaaatgaaatgcaattcagaagtgaaagagacgacaaattcaatccgaagtaaaatggagtaagaaatgaaaatcaaaatgcaaacggtcctcgcggccgtttctgtgttagctcttgccgggtgtgcctctcaaagcgaaaaggaaaaacaaatctctgcacagcagctggtagatcaggaattgcgtgagcaagccattaagataaaactcgcacaggatgagctttaccaggctggagccattaacaggacccgttacaaattccctaccattatcaatgcaaacagtcagtatgttcgtgtgagctggcagggtgatgcttacgaattactggagcaactggctaaacagcgtggccttcaattcaccagtcaggggattaagttaccgcttccactcaatatcgatgttggtggttcgaaggttacgtttgaacagttgctgaccctgatccgacaacaaacgcagtaccgtgccaccatcaaccagaaacccggtgaactgcagctgctttacctcacaccatcgaagaaaggttcattcctcagagggacccgcccatgaaacttaaactgatcgtgcttctgacgttaccgctgcttgctgcgaacgttgcacgcgcggacgactccgatgttaaaggcctggattactacatggacccgccaagccaaagtgacgatgaggccgatgttactggcagtatgctaaacgacgcagcgcacaccattggatttcgcggcggtaaggcagaacgcgctaaagaaattcgcgccgcgctggaaaatcagcgcagcaaccttgattacatgtattcgttccagccgctgatctcttctgaaggctacctgcctccagtcattactgaagctaaagacgttgcacacattaccaatgagcaaatccgaacggccaaccgtgcctatgacattgtggtgccggcgcgctttgtcagtaacccaccgacatggaaaagctatcttctgaccgggctgatggcgcagcggatcgaattgcctgaaccggcggcaatgccgaaggacgggaaacaacgtgatatctggaaaaaagctgtcgcgctgggctggtctgacggtcgccaaaaagctgatgagatattcactgctaatttcaatcgcctcacacgggattacaccggcatgttgcgctacagcacgctactgcagcagggcatgattaaggctccagtcatcacacagcagcagcaaacagttacaggggataaaaaccgcctcatgctgggcgataaaacgaaacgtatgaagcagcaagctgagtttgacatcaacaaacgcagctggaaaccgaccatacgttaaggggcagtagatggaagcattcaattttggtccaacgctgacgccccagacgctggaacgttttctcgtacactgctcaaacaatgaagtgtccgatattctgttgcagggcggagataaaatctgggtagaacgtcatgggcgccagttgcccatgacatcttacccgctcgacaacctggagctggaacgtaccgttgataaggtgttcggtcaggagatcaaagggacgctgaaaagtgctgcggtcgttaataccgctatccagctaagaggtgatgaagcgggcacgattggactcggccgcggcgaaacccgccggttccgcgcgaactttactcaggccgatatttataaaacagaagcggcaatgtctctgacactccgtgttctgaatgaggttattcctcagcttaaacaaatgggtattgagccggacctgtttaattctctgttgccggctgccggcctcggactgatatgcggcgagacgggatcgggcaagtcaaccttgatggccagcatattccagtattgcggtgaaacgtaccaggaccggaaaatcatcacggttgaagatcccatcgagtatcgcctgggaagtcgagactggattgcgccggcgccggcacagtcacagattgggcgcgatattgattccttccacaattttatcactctctccgggctccgtcgttctccgaagattatgggggttggtgagctccttaaccgcctgtcatttgaagctgctgtactggcaggtaaatccggccacttttgtcttggcacactgcacgtcaaaacggtcggagaagccatttcccgatcattgcagaactatccgccggaaatgcgtgaagctgccgcctttgacctgctgagtatcctttcttacatcatcgtgcaacgcttgcttaaaacaaaagacggccggcgcaaggctgtccgtgaatatctcgtcattgataacagcctgcgccgcaaactctatgacgttgattacagcaagtggggccgctacatcgacgacctgttgatcgctgaaaaccgcagtctgattcagaatgcctgggcaatgcaccaggaaggtttgatcgacgaagcggaagtcatcgaggtcgcaggatatatggactatctggcgttaaaagagggcaagtatggacattaacaaatggcgttatgccggccggccactgacggtttttggtgtgcctgttatctcgtttcttgtgtacttcatctggtttccgttcccgtcagtaaaaacctttgttatctgcacctgtgtggttctcttttactttctgctggccatgatgggctacacgttaccggtgctgtaccaggtcattctccgggttatccgcgggaaaaaacttaccgggcggccgtggtggtaccggcgctctcagcgataacacataactacaggggtagacagtatggcgaaacctgaaattgcaactgaaagaacccgtctgagtattccctttgacgatcgccaacgtgcaatacgtgcggccggcaaactggctgatggcagtaatgctctggactacgtgaaggaagaaaaggtgtggtatgcacagccgggggccaacctgagccggctgaaagagtggatttttgaccccaataaggtgatcgaagaaccgcccctggatattcaagccattgaagatgaatttacggcgtggcttgaagagcgcggtgccattatcaaagaccccatagagttcgatggccagaagcattatgtcgatacagtggacggtaaagctggttcaagaaagggggtgtatgccgcctttctggatggccgacctgcagggtggtacagggactataagaacggtggtgaaattcagaaatgggtatctaccggcgcagcccctaaccctgaacaaatggccatgctccgtgctgatgctgcggcaaggcgtgaacaaagagctgcagcgcaagcggataagtttgatcagaccgctaaccggttgatggaagaatataaccggcttccggatgccacgggagatctggcatacctgaaaaacaaacaggtcattgccgctaacggccttaaagctgatgagcggggcaacgttgtgatccctctgtttaatgctgatggcgagttcaggacccttgagcgcatctggtccgacggtagtaaacaccttgaaaaagacggccaggcatgggggagttttttcgttgtgggtggccaactgaaggatggtgacaacctgctttatgccgagggatatgcgacagcagccagtatcagcgaagccatcaatcagcctgtgatcatgacggttaatgccggcaacatggtggaagttgctggccgcctgaaagacacctttccaaacagcactcattacttccttgctgacaatgacatttataaagacgagaacgttgggctagaaaaagcgacagaagctgcagagctgactgccggccatgttctggtccctgcctttagcaacccgaaagaagggcttactgactacaacgatttgcatgtctctgaagggctggagcaagtcagattgcaggtagaaggagccattaatcaaatgaacagggtagacactatgccgactgataacccgaacattactgacgttaatcacagctcgactgacagcgctgccgttgcagctcctgaaaaagccgcgccggtggccagcacccctgcagctgctgaaccggtggaagccgcgccggtggccagcacccctgcagctgctgaagcggtggaagccgcgccggtggccagcgcccctgcagctgctgaagcggtggaagccgcgccggtggccagcgcccctgcagctgctgaaccggtggaaaccgcgccggtggcaagcacccctgaagctgctgaaccggtggaagccgcgccggtggccagcgcccctgcagctgccgaaccggaatacgtcgcaccaacagatcgagacggcgatgatgaagagagtaatagcgccagtgatcttgaggcgtatgccgctatgatggcctttggcggtgaaacaactgacactgctcagcaaaagcctgaagggatagcgcccagcgaacctgtagctaataccgccccggctgaaaatgaaaccgaggagaagaaaaaaacaaacgaagaactggaaaacggcatacgctgggcaaccaacgataatgcagaaacgccaccaaaagaacgtatagacctggaagggttaattgcccggtttgggtacagagcagagaaagactatacggcgtatacgcttgatgggcaggatgcgttttacgattacggttcacatctgcgtatggctacccctgaagccagccagagtgacgagatgatcctggcggccgttcttaccgcggataagcaacatcatcgtggtattgaaatcaccggcagtgacgaatttaaagagcgagtgtttaaccttatcagtgaatacaacattgaggtgaaactgactaacccagaacaaagggttaaattgctcgaactcaaaaaagcaaatgaaacagcaaaagaaacggcaaaagaaaatgttgctgattcaaatgcgaataagcaggaaggggcaaacgaaaaggcaaattcatcagttcagcagaatggaatgcgctgggaagaatcggcctctgtacagccgaagaagcagaatgcatctgatactcaaaaggaagataaagctgcgggtgaatcctgggagaaaggcgttatcgtcgcgcatggacgtgctaagtatgagtggaaagatgatgaatccatgaactactttgtcaccctcaaaaatgaacatggagagaagacactctgggggaaagaccttgagagagcggttaaagatgctggtgtagacaccgaacgtcttgttacagtcagaaagctgggatttgtggatgttgaggtgaatgcacctgtccgtgatgaaagcaacaagatcgttcgttatgaaactatccagaccaaacgtaatgagtgggaagtaaaacctgtatacccacagccaacagcagggaatgaacaggcctataagccttctgaactggttccgtatgatgctgcaaccttccagaagctccgcacaacaattcagcagattaccggtgttgatctcagccgtgaggccgtgcctgaaaaagaactgtattggttcctgccgaatggtaagccagctcctgaaagcatgacaaaacccacaggctataaactccctcaagagtcaaaaaccgcgggtacacctttgctggtcgcaccgcataaaaaaggcgaacgcccggattattaccttgttgagtctcgtaaaggattcatgcaggggatcgcaaaggataaggaatctggcgagtaccatagcgtaattggtaaagtgaacaagcgtattgataaagatggaaactggaaaacgtatatgacactttcagcaatcaataaaaatgcagaaagtggaattgttttgcacggatacggccatgttgatcagggaggtaaaaacctgctttacaaaaacacaattgcaaatgaaaaacagcctcagaatttacaaccggcctcagatgaggtaaaaaacaaaacagtaatgaagcagctttttcatccttctaatgctgcaaaaagaaaagatgatgaaaggagagaacagactcatgcaccagtcgcaaagtcagcgcctcggccataaaacaaaaaccctactcgtagcagtagggttttcctgcatttctgtgagctcatttgcagccacacagacctgtgcagcatacgaactggcaaaagctggtgctgatgctgcctataaacaggaggttgaacggattaacaacgccgctaaaagtgaatccagtacatctgattctctcggacagtgcctggggtcgctatcggtatcactaaaaatccctcaatttccgtctatcagcgacatcattgataaggtgaaggatgaggtatgcaatacggttaaatcgaagataaatgagaagtgggggagcctgactaatacaacgctggacccgtggtcaacgatttcatcccgactgccagatacgtcaggtatcctgcccaacgcatcttctaagaatgtgcaattaaacaatccggcagctgcgagtggaagtggtgaggatgcaaacagtgatgcattcccgttccatttgtaactaccggaagagtcctttcgggattttatcgaatagcttgccggttcgtatttcaacgcggcggtgttccgccgcggttagtaaacgcataaattctgtggcaggtagaacatcacgttttgagtctacatacgtatcaagcgaatcttcattcatgaaacgtctgacagtatgcagccagtcttcagctgatttcatattttttgagctcatcgtcgctcatctcggcaaacgctgcaggtcggcgaccttttcctgtccaggttatggactttccatcaacttcgatccggtacttcacttttccaaaagtggatttacttttcgtggacggaccaaagagattttcgaagatctggagagcttcatcagccgtaatattgtgttcttccatgaaccgctttgtggctgcggctttctcttcagtggccttctgttcttccttaaatttctcgatcattccctgtgcaatgtcggaccacatattaatgagttgttcaaggttttctggcgggattttgcgcaaaacggcggtggcttttcgtcgttcgcacatgataactgcacaggattcgtaatcattatgttcgctcatgtatacctcattgttcattaacgatctgcgtgattttctaacacaaacttttgaaaagagaaactctatgaaaacacaaaatggtgaaaatattatggatcaaactgactcggatcaaaactcagtcgttgatgatgacagcaacgcccttgctccagctctggctgaagagagtaaccgaacggtacaagaaatcgtatcgaaacatggtttaaaagtaattttgattcaaacgctaatcatattagtcgcaatcattatcattggtcttcagggttactggcttcaccagaaaaaaatcaaatatattgcaacgaatgggggcatgatcacagaagttcatccaaccgatgaacccgcttacacggcggaagaggtgatggactttgccaaccgtacaatgatcaaatcgctttcactgaactttgtgaattacgaaaaccaattaaccagcgtccgcggcgattttagcgctgaagggtacgttagtttccgtaaagcgctgtccgatagtgggctgctgaaagatatcagtgaaaaacgtctgaacgtaaaactttccacaactcctggaaccctgatcacggaaggtcttatccgtggcaccaaaacctatgcctggcaataccgcattccggtgactgttcagcttgttgggcaggctcaggactacagaccgcaaccttacgatttgatggttcaggtacgccgcgtcaaagaagacgaagatccacggggtattcaagtagcacaggccattctcaaaccccggaactactgaggtcaatcatgaagattaaactgttaactgtacttatggccgcactgttttcggcaaattctgtatctgcagctgatgcgccgactggtaatggagcgccgcagcaaacggcgccagctgtgcctggtggtgatattgctcaatggcagcaggccagttccgggttgcccgtaaatcagcagtcacaggtgcaacagcctccacagcaaacagcacaggaacaaggtcagcagcaggcgcaaggccaacaaccacaggcacaggcccaacagcggccaacgactgcactgcctcctgcacccatgccgcagattcctccaccacagcagcaggttactcatgttaatccgcagacactggaagagttacctgcacctgttctgcagcagatccagcctattacccctggtcaggtgcggtcggcccgcagcgcaatggaagatatgagcgcggcggccaaaacctcaccattaacgccgatcccacgaatttcaagccagacggtatcactgtcagctggcgcatccattccgcaggtacgggtatttccaaaccaggcaacgacagtgacgttttcagatgcaaccggacacccctgggtattaggtgcccctccatataattcgagtccctgtgcttcaggtggtgaactgtgtgttggctatattccaggatcggcggtattcacaatccagccgaccaatgcctatgccagcgggaacatcacagtgcttctgaaggggctggccacgccggtcattattaacgtgaagggggcagaaccctccgttaaatcaaaaaccgttgatgttgattatcgtctggatctccgtatcccgaagcgcagtcctgacacaccggtttataccgctacgcctgagaaaaaaatctcactgtatgacaagcagctgcagagcgtgctcgatggcatcccgcctgaagatgctcagcggttgaaacttaaaaacgcgccagggagtaccaaagtctggcagatcggtgatgagctctacgtcagaagtaacatgcagctgcaggatgagtttgaaaaaacgctttccgctatcgatggcacacatgtttacgtgctgccggcaacaccagtgctgactttttctgttaatggtcaggcgcgtgacgttgaagttgagctgaattaaggggaacgacgatggctacagataaaggtaaagaaaccagaaaaatgatgctctacgtcggtggtgctgctgcagtggtggcgcttctgggagggtactggttgtttggctcatcagatgagccggcaacgggcgggtcgcaggtgaaagcccaggggctggaagggaaaactggcagggaaaaagctaatgatccccaatacaacaagctgctgaatgagtggaatgttgataattcagataaagcggctctcacaggggatagcttcatctccacgttgtcagccagtaatccggtggccgttgattacggcaccaaagcaccgccaccaaaatattcgtgggaatcgtcagcaaagccgtcgggcgataacagtaaggcgaatgatgctgagcagaaagagcgtgagcgtcaggctaaagcggtggctacgttactgcagaaaatagaccaggccagaacaccagttgtcacgggtgttgctcttgcaacccctctgggagcagaaggaaatgacaagaacggcggtaccttctcagggtggaccgattcggtctatccgcagaacaaaaagccggatgctgaaaaacaacagggggcgaagaataaggccaaagataaaggagcccggcttgtatcggcttacacgcttgtgccggcagtaatggatacggcgatggacagtgacgatcagaacagtattgctattgcccatgttccaactggagcgcaggccggcgccaaattctattctggccaaaaccgtcttgccggcgatggtattcgaattcacttcaccggcatggagctgaacggagtgcagtgtaaggtcgatgcgtatggcgtggcttccgactcgttgcgcgcggccgtttcctcaaatgtaaataaccgctggttcagccgtattattctgccggctgtcgctaacggattaggccgaactggtcagctctatgcggacagcaattcgcagatgattatcactgacggcgggaacgcatatcgttcaactgatacacctgatggcaaagcggtagccggtaccattattggcgggatgggtgagcaagcagggcgtgtcctggccgacgatgcagcccgcctgccgattaagcaggttacggttgaccgtaaccaactcataggcatccagtttgtcgcgccagtgtatgaaagcgactgtggcgagaatttggaaaatgcttctgcagagcaggcgcagcagcaagcaactccgacactatctagtgtgcaacctcagcagatgcctcagcctccggctcctaattaccctcagcaaaactttccaagctaccccggctacgggtatggcaccaataacccgtattaccgttaaggagattgataatgaatctgtttgaagatgacaataaaaaagaagcagctcctgcccaggaaccggtgtctgctaagactgctgaaaagccaaaaccgacagtgacgccagcagcaaaagcaaacaaaaagccgcttaagattagtgactatgcatggattggtggtatcgctgtcatagctatcctgcttttagtctggttgtttgggggctccgatagtggcactgcgcctgcggccgggaatggcacgatccagtcttcagggcaacgtcagaacgtacaggctgcccctgccgggcaggattctggtgacttccgcgagcagatttccgggattttgctgcagcaaaaggaacggctggacgcgatagaatcaacttctaaaaatggaatgatgatcctttccagtcagttacaaagcgctaatgagcaaatcaaaaatctgaacaatcaggttcaggaactgaaaatgaaagccgctggttatgggccttcatttagtaatcagagtggaacaatttcgggggcaacgtcgcaggcattacctttgttgagaggtttttcaattaatgatttatctggcgatcttgcttgggtaaaatacaaaaaccagacgtatgctgtgaaggtcggtagtcaacttggcggagtcaccattacgggtatcgatactgaaaatagaatcgttactacaagtaaaggccttatccgctaaacaggacctaaaaaggcgaaactatggatttagaatcggtattaattcaggttgctaacggtggtcaggaaccgctgacaaggctaatcataggtgtcgctgcagtcgttgggatcattggtgttattggatatctgatgagcctgcggaagcaggccaggcattcaacaaaaggtgtggaagttggcagaatttttgccggtatcatcttctgtgtatgcctcatcacgttgaaggcacagatgaactcaggcggtgccactttaggatttggcgatgttagcttcggacccgtcagttatgcgagcgaatctacgtttggcatgggagccgcagcagtcaacgcagtgctggggattgttagaattctcggcgcatactttttttacaggggtataaaggggttgaaagactcttaccttgagggccatacggagctgtcagcttcgggtacgagaggggcatccattgtgaaaattgtatgtggactacttctgatgttcgatacacaaacgctcgatgcgttgcagagcacattaaacattcactggtaatcaaaaaggatactttatgaaaattgttaagcttatcaaagacaaggcattatctgcatccatcgcagcgtatcagctgcgcggaacggtggcaaaatcgttagtggtattgccgctggcattactgagcaaagaggctgcggctgaaacgactgtctgggagaaaatcaataccttcacggaaggtcttgccagtacccagcctggtctgattaccggcggtaaagtcattggtgttgtgttctttattgtcgggcttgtttctcttttccttaaaaacaaacgcgggaaagatatcagtggcagcttcatcttctggtcaatggttgttggtgcttgcctggtcggcctgaccgcatggatctcttctgttggttcaactgttggcgttgacgcaaccctgtaatgtcccttcggccccgccttgcggggccaatatcggagttccctcatgaaagattttaagctcaccaggaatgaacaacctgccagatgccatgtgtgcgggataaagagcaaagatgttttctccctcgcagaagatggacagaaagagagaatgctttgctctgtatgcctgtcgctcgaacatccggagaaaatgaaactagagactcaaaccggacttctgctgaagagaaatacaaaccgaaaactctacagcctgctgtttcgttcaattgcaaaggcggaaaagtcaacggacgattcttaccgtgaaaaggcgcagaaacttaaacagatattgctcagcacaactgacgtagttctaaacgaaaccggcagtgatgatggatacgatttactgcacctgataaaaaaacaccatcagttacttcccatgaattttgacgatgagtttgtcgcttctgttgcagagtcagacgaattgccggatgtaagtgactggaatgaaatttacaagggtaagagaggttttaaatgatttctttctttgaggatgtgatcgaaggtatctcacgcaatctgacatcaaatgactcgttaaaatactgtgatttgacaacggttgtagggcttacgtcgcaggatcgcgttgagcatcctgaaatcagggctccgtacattctgaccactaagaacaacgattttcttaccgttattgaggttcagggcgctaaatcatcgtttgacgagaagtcgtttaatgacttcatcatgcacctatcaaactccttgtcgaattcctttattaacagcggccataagttgtctgtggtgttcgaacgcgactttaccaagaaccaggaagagctgaataatgtttataaaccgcaggtagcagcaatagagcgtctgcagatagacattaaagacctgatcgccgatgacgcaaaaaaaatcgccgctcattacagccgtgaacgcgcttacatcgtgctgtatacctcgaaggggatgattgcgaaggatgaacttaaaaacgaacaggcaaaagctgccggcgtactgaaggatatcccgcagacgcgcttcagtcagaacccaatcgaatttcagcttgaaggtttgaagatcacgcatgacgcgatgctgtggcgcctgattggtatgcttcagggggatgatgaatcaggaatggggcttctggttgatgtcctcgatgtcaaacacgctggcgcaatgatgcgtcagatgctgttccggaattcaacatcagaaaactggtaccctcgcacgccgtttgaccaaaatctgcgaatttatggcgccccacggaaagacaatatcgactccgtgctgccgccgcggctgaatatccagatcatggaaggggaactggaagaagatggtggcctgatttactgtgatggtaagtattacggctctgttgcccttgagttaccaccacttcaccctgtcaaatttaagcagttattcaacgcgattaaccggaaaatcccgtaccggataaaatatgactttataggcgggggcaaaaaagcactgttctggccatcagtaatcatatctttccttcgtttcattccctcactcggtaatatcgcgcgtgatatggattatgtccgggcccagagcgaaaccgatccaacggctatcatggccatcaactttgccacctggggcgacactaaggatgaagccaaacgcaacctggcggctttaactaaggcgattgaaggctggggtgtatcaggtgtgtccaagacatatggcaatccaggtagcgcactgattgcatccgttccggggctgacgacggccacacctggctcattgcattatccgccgttgagtgaaggcttgcggatgttaccttttgaacgtccggccagtccctggcatggtaaaggcaatatcaacttcatcacgctcgatggcaagctgttcccctatcaaatcgccagtcccctgcaggagaaatttaccgatgtcatcaccggtgttcctggttccggtaaaagcgtgctggccaaccgccttaacctgagttcgatttaccgggctgataaaaaactgccttatctgaccatcattgataagggctacagtgcaaaaggtatcgccgatctgattagcgctgagctgccgtcagagaaacgtcaccagattgccagtatcaccctgaacaatgacgaatcgcactgtattaacatctgcgatacgcagttaggttcaaggtatctgacgcagtttgaggaagtattcttaaagcagatgcttaacgcattctgtattgatccggcaattggccagccgccagatgcaatgagcgttggccaaatcgtcagtaaggttgtatcaaacgtatacaaagcaaaagcgcattcaagtgctaacttgtttaagtataacgacccggtgattaatgaagctctgaaagaatcaggtctggataaagagcttggtgaagactggtttgaaagagctacatggtgggaagtcgttgataagctgtttgccaaaaaatatcttcatgctgcgacggttgctcagcgttatgctgtgccaaccattcacgattttattgctgaactacaaactgaatcatttaaaaaccaatacgctgacgtaaaagttaatgggagtgagcctgtagtaggcttcgttgcacgttgcttccaggctgctgcagctgagtatgccattttctcagggataacggtttatgatttcagcccggagactcgcattgcgatcctggacatgcaaaacgtcctcggtgacagaacgacacctgcaggtaagctcaagtccggcattatgtatctttttgcgcgccagatggctgtgcgtaactactacctgcctcagtccgcagaaaccttcattcctgctttaccggagcagtacagggcatatcaccaggcgcgcatcagggagctttcagaagaggttaaacataccttttatgatgagtgtcataactttgccggcatcgactttattcaaaatgcgctgaacaccgctgaccttgaagatcgtaaattcaacgtccgtactgccttctcgtctcagtatctgagccatatgccttcgagtgtgcttaaaaccatgaacagcctgtttatgatgcgcctgacggaaggggatgaagagtatctgaagcaattagagatcaacatcccggccgatatcctgcgtcgtttcagacagcttccgcaaggggtttatccggacggaagtggtactgcattcctcggtatttttaaaacaaaacgtggtcttatttgccacattctgaaaaacacattaggaccaaaactgctttgggctttgaactcatctgcaaaagatagagcgctcagagatgtgctttatgaagagctgggaacgaaaaaagcaagggaagaattagcaaatagattccctatgggaagtgcatcaagtatcattgatgaaatggtggtcaatcagggtggtgagcgtgattctgaagaagaatcacaaactatggcgatgaagctcgctaaagaaatcatttctgatgtgagaaggggattcagatgataaaaatcggaaaagcaaaacggtcagtgatggccgtttcaattctggccttcagcgtacagtcagcatttgcctatacggtcaacgttaacagcagcattcccatctcaacgcaggttatgccggcgttgagcacagctaatggcctgttaggtaatatccaggccacgctgattaacattggtacggcaatcagccagcagggcgatcgccaggcagcattaatgcagcaggtggctgaaactaaccgacagttcgaagtcgagcaaaaacgtaatgaccggctgctgcaggcacaggataagtaccgtgttccggatgatatctgtgcgcagtctattggcggtggcgccgcgggggtatcctctgcggccgctggcggtcagcgaacgctgggaagcgccagttctgcgaaagatgttaaggctatttttgaagtgccggcacgcgcaaccgacgtggatgcatcttcaacggccgaagttcacagccagtactgcgacagttcggattatgctgcatttggtgggacttcattctgcccaagtgtatctgacatgccgcgggcagatacgtcgctgagctcgttgctttatggcgccggcaaagaaggcaaggcacctgatctgacgtttacagatgagcagacagatgcggcgctgcgctatatgaaaaataccgcgttgcgcagtgctggtcgtcagctgagtcgtggggaagttaaaggcgccagcgggcgaatttacctgggcatgattcagcaataccaggcgctcaccgacgcggccgcacaacctcagatggaaatgatcgccaactccaaaccgaacccggccactgatgccgcgcttgcagatgcgcgcaaagtgccggaagtcgaatcctatttccaggcgacggccagtaacagggcgaaacagcttaaccgtatgtctgccagagagtttgaggaattcgaagtaggtcgccgttacagcaaccctgcttaccaggcgacgctgaccaacaagaacaccgaagatttgatgcgtgagcaaatcaatatcgccaatctgaacaactggttagtgctgaaaaccaaacagcagctggagaaacaaaatgtgctgcttggccagatcctggcgaacgacgcatatcaaacatacaaaccgatgctggcagcacagctggagagtgtgaacgcgggagtatccagaaaatgaccgacgaaaataaaacaggggataaagacacagccaaatcagggaaattaaaaaaaggccttgatgttgtaacaggtgtcaatgacctcccggaagggaaggctaaacgaacgatatattatattactggcatcagcgatatatattttattatcgcgtcagtaaagcaaacattcagcctgctctttcagcgggcttcattcgttaagaagcaaataaaaaaccttgatggtccacctgttgattctgatgcaaatcagccgttcgccgaagttatgaaacggagcaatagacctgtatcagaattgttggataaggccagtttatacaagaaatactggctttgttgtttttttgcattagtcctcattctgctttttttaacgtctggttatgccagattgcttctaaatggctcaccgaacatgagcttgttaagagcaactctcacctgtggcgtcctgtttgctgctgggatctttactttcattaaggctctcacatgtgagttcatggggtggcagttacgaaaccaggctcactctgatgcagagcaaggtacgttacgttatttccttaatgatgggggagtgcgtaatacatttaatttttcgcaggctggtcaagagcgggggccacatgagtaaattaaaaaggttgcttctatctcttttcgttgtggcgtcgccttcatattcagcggatttggggattgatggcataactgaagcggctaaacgttcaggtgacctatcgcgtcaaatgttacataccgtttttggtgaggtagttaataatccgtttagcccttccggcgatggcttacttaataacgttttcttcactgtcaacgaagttatcgcgattcttgcccttgtctatatggggataatcagcattaagaaacttcaccaggcaggtcagttgggaagtttcattgagggtgatggcaataacgcattcaaaattgttaaaacgacttttgggtggttgttgcttgtacctaccgttgtcggttggtctgtggcgcagctgctttttctatggtgcggttcaatcattggcgtaggatctgccaatgttattgcagataaaacggcttcggaattagcgagtggtaaagctgtctatatagcccctgttatgccagaaatggcgtctgttgcaaaagggatgtttgaaacaaacctttgtgctttgggtgtaaatcaggggattgctcaaatggaggcttctggccaacattatgagaataacgccagaatgcaataccaaacaggtgacagcacatattctatatctgtaaacaacggctcagctgtatgtgggaccgtttcgctgccacaacgtccagcaggatggacttctatcttcgctagcggttatgcggattccgtctacagcgttcagcaacaagcaacggacacgttgtggggaaaaatgaagactgctgcagagcagtttaatgcagcatatatggcaaaaatgcgttccggggacggtgagctgccagatgttgaaagcgcgatccagactgctgcgcgtgattaccagcttcaggttcaacatgctgctaattcagcagccggtgacgacgaaattgttcaaaaaatggagtcggatatcaaagagaaagggtggctatatcttggtgtgtactatcacacattagctactgcgaataccgagatgaaagatgttgctaacctgaagccggtagtttcaggaatgagtaacgatggcgatatcggctctatcgattactttaaaggcttgttccaggcttaccattctcaactgaaaaatagtaattatacgccgcctcttggcaccgaatctaacgcacttgcaaggaacattgatcaaaaaagccttgaaaatgccgccacaactggagacggaacgtcccttattacaaaaattttcgacttcaacgttacaaactggcttgcaacgagtaactacggcactggtgatggttacagtgacgtgaccaaccctttactcaaaatgaaagctatcggcgattacacgttaggtgtagctgaggttggactcgccagttggaccgctatcaatgttgcagtgaaggtttcagaagggaagagtctacccggttttgctgcgggaatgatgaacaaacttacagggattcgagatgctattgctggcgtattagaatcggtttcgttcttggtttacccgctgttttatagcctctttggtatcggtcttgcactatctatctggctgccgtttataccgatgatttattggtttgttgctatggcagattggctggttacgcttatgacaggtgtattggcgtcttccctgtgggcagctacccatatcaacattggccagagtaatgatgagcggagtacatatggttatgtgttccttatcgatgtaatgattcggccgatgctcatggtaatggggtttatttttgcttctctggccatcgtagctttgggaacagcgttaaacatgatgtttaaattcgctatggaacaggtccagtctgactctttcactggcctgctgtcatcgatcggatttttgatgttatatgcaaggctttgcacaggtatggtggctcgcgtatttgcacttcctgcgaggatgccgaactatgttataaactggattggacagaagatgaacgattcagtgttgggtgatatgcagaaccatgttcatgacatctttgctgcgttcggtcgtggagccaaaaatatgaaccgtaacaaccctaagcaatttaatcctgacgcaggtgtagataagagcaaggacggcatcaaaggagcttaaatatgaatgcgttatcaaacactgactttaagaaaattagtaacaatgcgcgtcctaaaaggcttggatactacgcatcatggctctggatgatgtttgtattcctctttctgttcgttagcgctttcgtgtttctgagcggcttatactggacggttagagacggttcaattcaggagcactggtttagtacagtcggtatctttgtgatcgatgcagtgctgatctggatgttgaaaaaggacttcaggagccgcaagctctaccaagtaactgcttcaatgaagaactctggtttcttcgagcctcataaggactgcgagcactatgacctggcccggagaacttacatcggctttgacttcagtactggaatcataggagtagcatccctgtatgccacatccagtattaaacgtgagcggctattttttgaagccgaaacagtcgagtcatgggagtcagtcggcagggagttaatcataaacctgcgaaatacggggcttacgacgataacgatcacagctccaaatatcaataaagcgtacagggatatggaaatcatctgcagaacgcataaaaatagggatgaaaactatcagcatctgaagagtaaactgacagacgctggatggttcataaacagcaattattgagtttaacccgccgaatatggcgggttttttgtgtatactcaagtggttatagtcgtatgttgtgggaggaagtagtgaacagagttactgagcgccaggaacagaagagtaacccggagttccagagagagtaccggaaccgtatccgggaaacacacgaccagttaaacctttatctccccaaagatgcaaaaagaatgttgatagagatcacaaaagttaaaaaactcactcaggctgaagtcattgaatttcttgtcaaaagttattacgacggattgaaatttgacgattctgaagagtgatcttactatgtaaaagcgggttcgcaggtattgccccgcttagaaacgcatgatatgttaagcggcgtttacataaccttacggggttattacagaatttagcacgggaagagcggcccggagatgtaccgcttggggtgtggcaggagccacaaaaagtaaaaccccctgattcatcctctcactttggccggtcagagatgaaatcagggggcgcaaactgccttgctggcagaaaggattatagcaaatgcgtaccctattgcaatacagcccggccaataagtgcaggggctggtgaatcaaaagaatccatacctacagctttctgacattgagtcagtagaaggtctttcgccggagttcatctcctggcttgaaagccagtcccctaaagaatcgcccctgcagctgcttctcgatgaaggcaccaggcccgtgcgcaaaacccgccgtcgtcgcggtacgcactcaacggcgtgtctctgcccggagcccagctggtaccgaccggacaatttcaaaaagctccctggccagcttggccatgcctacaaccgccttgttcgccgcgatcgcaaaacgggcgcgttaagtctgcgtatgcggatcagccgccacccgtacttcgtgcagctgcgcgagcaggctggccggcagcgcgacttccgcccggagcgggagaaattactcgatgcgattgtgccgctcctggtcagcaccgttgaccgggctacccatattgacactatcaacctcagtaaaatggcgtggcagctcagcgaaaaggacagcgaagggaacgtgatccggaaagtgacggtaccgcgggtttgccggctgctgcagcacatgatggaattcgggttactggcgctgcctgaaggcgtcacatgggacccgtttaaccgcaagtggttccccaagcatgtggtgctgacggaacgcctctggaagatgatcggtgttgatctcgataagctgtatgcggagcaggccgaacaggtggccgctgaagctgcaggctggataacccgtgatgaacatggccaggcggaagaaatctctgttaaagccgcgcgccggcgctggtatgaaaaaatgatgcacgccacgctggttcgccggcgtgaagccgccctgaagggtaagcgcgagaagaagctgaaagcgctggcagaacagccctatgacgaccgggcttacgccatgtccgttcatctgatacggacgctgccgaaagacgaactgcacgcgctgtcgcctgagcagtttacccagcgagtacgcagccacctgtatcagctcgatctggggctggagagggaatccggtccccctgattatcactgattttctccccctgtaattctcttaccgccgcctggcggttttcgtgctggccagccggcgcgacctgctcaaatttcctccagttttaacgtgtggccaacgtgattttttgacgttctggcgccgccggtggataaccggcagcgaaatcaccactatccacagttatccgcaaaggaggaaataaagtcacctgcaatcgtggtcaaaacaagctgcaatacactcttaacttctttaaaaagatctttattcaataaagagatcactgttattcaggctcaattttctgtggattatgggaatatgcctggcctttcagcgccgctacgcggcacgacggccagaatggtcgctatgctcccaggttagcacccggcaaatgccgggcagggtggccagcagaaacctccagtccggattcttctttgccccaccgtgcttttccccgccgagccaacggctcggcagaaaaaagcatgatggatgttgtaactatgaacacccccgtaagacggcaggggtgcgacacgcccccctgacgtcaacgctactacccacgcgcccggtgtgccgctaacgtcacaccgcccgctcgcagtagccagggggtaatgtggccacagagccatctgcagggcagtctggcaggataatcgtctgcactgacgtgcaatgcctttcaggcaatcagcaccggtgccagctgcccgttttaaaattaacagggaaaagaggatcgatttagccgcggttatccacgtcagccgctctcaggcgcgttaaacaggggagcctctacgccgacctcattcaacaggtccagggcgacgctcttggcaccgcgcttaactatattttcaaaacagcaatggaaaatgtacaggcagattcaacaactggcctttggtcaatgataggtatcctttttgtctatgcaaggatgtgtacaggtatggtcgctagggtatttgcattacctgcaaggatgcctaactacgtcatcagctggattggtaacaagcataacgattcaattcttggtgatatgcagaatcatgttcacgacctgtttgctgcatttggtcgtggagctaaaaatacaggtggacgtccttctgggccgaagaacttcaacccaaccaatacggttgataaagacaaagatggtatagcgggggcgtgatatgaaaggatattcgtatactgttgtcgataacaacatggctaaatcaaaaggtgttggctactatctgtcacgcctgtctgtagccataagtatagttattttaacaataagtcttccgttgactgtatattctggctttcttactttaggtggaagcctggcattggcgtggtttgttgctctacttgttttcgatgccatgctgatcacatatgttgtacgccaggtaaaaagtaagggagtcaaaaaggtcgttgcaggcatgagagtttatggatgctttaatccatcggctaaagaggaacaatacatgccggctcagaaaacatattttggtatagatatggactccggtgttgttggtattgctgccctttatgctgtacaggcctctattaagcaaaagagaattttgtttgaggccgagacaattgagtcttgggagtatactgatagtaaacttgtcgtaaatattcggaaccgacatgtgcctactgttgaaataatcttgctcaatgccggcctagcatatcgccagctggaaatgttaaccaggatgcatgagcaacataaaaaatacaggggtgaaaaatatctccagtggcgaaagactatgcttgatgaaggttggatgctaccacgaacgtattgatgtttttccatagtgtatgcgatttgtttgacatgttcaactatgtaatgctttgtcaatgagggcaaattctaatcgaagaagttgccaatggaatggttgtttagcaaaaaatagtcaaactttcacagttgcattttttgatgccgtggtaaactggaggtaaagagtaagggggttgatatgaaacatgtggtcaatattcttctgctgggaatggtgcttctgggaatagccatgatggctgacacaccttggggacttggtgtggcgctggctcccttcggcgtttggggtgctcgtttcctatttctggttcacaaaagcctttgggctgctgttatcttttggggagggatcgcttacttccagtggcaagttgctttggttgtcggggctctgtttggactaacgtgctttatccgcgttgcgcggtcagcatataaagaggcaccacctacccggcgcagaaaaaaacaaactggtgggtttcaagattcctatgattttgaccagcgcttccatattggagctggagacgaataagggggcatagagcggggattagtgtggcgaggcgcaggtttccgatggcaggctaaacgcaaaaaatgcgctttttagccggtgatgaggtgaggccgggaggggatgcgtcgccgatcacagtttacaacagggtttgataacgggaggaaaagtcatgctgcagggattgaatcatctgacgctggcggtcagcgatctggcgtcaagcctggcattttatcagcagttacctggaatgcgcctgcacgccagctgggatagcggagcctatctctcctgtggggcgctgtggctgtgcttgtcgctggatgagcagcggcgtaaaacgccccctcaggaaagcgactatacccactacgccttcagcgtggcggaagaagagtttgccggggtggtggctctgctggcgcaggcgggggctgaggtatggaaagataaccgcagtgaaggggcgtcttactattttctcgaccctgacggccataagctggagctgcatgtggggaatctggcgcagcggctggccgcctgtcgcgaacgcccctacaaggggatggtcttttttgattgacgggttagttcagcttactgccggatttcaacgtgcagatccacagcataggcctatgccatgcgggtcaaggcgacttccggctacttttgttcaggtgcggagccaggaacgagaagtattttccacactccaggcattgaattttttccgttaattcttctttatcagccatcgtttggcctcctgattaacaacgccggcaaacgccggcgttcagggttacatctcgatcatcggaaacagatcgtcttcctggtggtaaagcacgtcgtctgtgtccgtcccatcaagccagtcagcgaacagttcaaacaggcacaccgacatttcctccggtgaaattgcgccacactgcagctcgtagaagacctcaattttttccagcaggctgttggccgctttgtcgtagccgaacagcttgatcggcgggaagtgcattgcatccgggttcgtttccgccagttcatcatccttacgttccggatagatgctgtacagcgtcgccggcgatggcggtaatacctcgataaagcggtttatccgcatcggcagttcgctggaaaacttctcattgtccggaatgtaaaacgctgcgctgcgtaccagggcatcctggaaagaaattaccccctgaccttcggtctgcttacgcagctcacgaatgttcacgcggtccttttcacggatttggtacagatccccttcccggaccgttttggaaaacgtgccgtgttcggcttcctgcccggagagctctgaataataatcctggccgacggtttcgcgcagtatttcaaaggttttccggtcttcaatcgactcaaaatatttcacgcgggtgttggcgatcagcgaatccacctcgccgttcgcgccgcgggcgagcgacgggtgatcctgggaggaaaatacgcccattttttccagtgaacgcatctgcgcactcagcgtatccataccgctggtgaaatactggcccagctcgtcataaatcagcccgtatggcgcctgattattcagcgcgtgagtgagcaggacttcctcttttttcccttccagctggtagcccaggtccttactgatggtcattcgctgcagcgtcacatataaacggccaagcgtggccgcttcaccgcgtgacagctccagcgacgggatcatgacgatgagaatgcggtcattgtggaaaatatcgtccatccggatatcgccgctgtcttccggaaaaacatggccataagtttcattgaacagggagagcgtcttcaggaactgacggctcatgtagttatgctgttcgaaggcgcgggcttcccacgtttccgggtattccgcgttggccagcaggaagcctggtgtattttccagataactttccagcgcggcgtaaccctcgctgtgccagccgtttttctttgcctcaacgtacagcgcggccattgccggcagcgacatgtttttctgaatagtgcgctgtgacaaaagcagttcgccgcgggcgcgcttataacacagggcattgatcagcgccgacatcatggcttttgccgtatcctgccactgtgccgaatctccgcccacctgggggagcatggattccatcaactggatgatgaaggtcggcggggcaacgctgaagaggttgactgagttggattgcgcggggatgctttcctgcttcaccaggttcacgaacctgtcaatactgccggtcagcaggttgaggacgtaataatcatcctcacggccgaaacggcgcgccagcgaccaggtggcgaaggccagcttgttatctgctttaccgtctgagaggcagtagccacggcaccacagcaggaagttaacgataaacccatagaaggtttcggttttacccgacccggtggtaccaaaaaataccatgtggcgcagcaggtccgtcatgttcagccacagctcgcggccgaacaggcgtccccgctcatacccgacatataaaattcccttcgctttctttcgctcccggaccttatgcgggatgcgaatgggccccagacggtattccgcctgggtttccgtggtcagggtgtcgtcgaacatttcgcagtctttcggcattcgcagcggtggccggaatttacgatccgtgaatgccaccatcagcggcggaaggatgaggatgctcagcagcatggtaaaggggagaaacagacccagcacggtcagtatggccagcaggccaaactgtacgttcggagtgaggaaccactccatgaacgaaccatctgggcggatgacttttttactgtctacagattcctgttgcatagcggctccgggttattctcctattgcctgctgcaggcgggcatcgtctttgacgacggaaaccgggatcacatcacccgaatcggcaacgatggacggggtgccgataaacccgaatttcgcaaatgccatattgttcgcgtcaaccaggcgggcgccggtggtatccggggctgttttcggatcgtcaattttcgggttcatggtggtgtatcccatgttaatcgtgtcttcccagcttttcagtcgggtatctttcccggtatcgagtacaggagcggcgcggaccatcgagcgcaggtcgccataaacggacacggggaaaatctcgacgttatagcgtttgctcagttcctgcagccggggttccagcgcgcggcagtgcgggcacatcggatcggaaaacacatacagggtgtgtttatgcccggttgaaagcgagatcgtaaagcgtccgctggtgacggcatctttcagtgacagcaagtcgttatcagccagttttttaggggcttcaggtgctgcctgaacctgcagctgctccggcgcagtgacaggctgctgcagacccggcgcgggagcatacgagtttgcctgcagtggctgtgccggctgatgcatattcatcaacgcgccgttgatggagacgataaagcccagcgcggcgactggccagaagataaagttagacagccggcgcacggcattgagtccgcgctgctgtttcagctttttacggatattgttcagcgcctcgatggcctgatattcgttttcaaagcggaacagctgcaccgcttcaccagaaaacgtattgtgcgtcaggatgtggccgctgccggcatcatggccaagtccaaaatgacaggctgacagggggaaggagtgcagggcttgttcctggtagcttccgtcagcggaaagataaagtgtgttgcgatcggtttttacggtgagtttcatacttcctcgcttagagttccaggtgatcgggacgggttgccggctgaggtttagccggcgtcggttcatcgtctgtcagtcgcagactgttgttcggggtgtcgtgcggagctgcaggcggcgtagccggctcgtcttccgcttcagcgtcagattcaaatgcaaatggcggtataacgtcttcctgccagtgagcgaccggaatggcgggatcgcggcggtcaatatggcggtaatggagcagatccagctcaaggcccagcacggccgcccgcacggttggcgggaatttcgccgccttttcttcatcgagcgtcagccggtaactttcgcactgactccaggcaataacgccacctccttcaatgaagaacttcccgcggccggcgcttgaaagtgcataccataacgtgcggtccacgcctttcagccaccggaaatttgcggatggcagctgcaggtcatcatcgaacagcgcgtagagtagcgaacgggagctgcggcgcccctttgcaaattctctgaacgcttcgcactggctggcgacggtccagagcccggcggccagcgaataatcagggaagccgtctttagtccgcaggcaggaacggttgagatcgtcgagaatggatttagccgttggccggtcgcccataaaccgcagatgtacaaacgaggctgcgagtgctttttcccagggtgccagggaaagggccgccccttcgggagccaggtttacctgtgacaggaataccttatgtgcggcttttttacggaggcggcgtttagcggtatcaatcaggttatgttcctgggcaaactcaacgggtgactgcgcgctgcgtgcttcttccggatcttcattcagcagcagctgatcgaggtggccaaaccgggcgaggatatgggctatccccggatagttttttaccatcacccagggcaggttccggatggtcagtttacgggtcagacggtagtaaggattgcgcgagataacccagacgaacaacccgatgattaaaatgaacaggggaaagagcacgctggccgtgtcgttcatcacgctgataaagtcccacagcgaaacctggtgattataccgcgccgccgacagcagcatgtcccggcgctcggtcgcattacccggtgtaatggcgtccggcagtacaccgtagatccagtaaagcaggcgacaggctccgtaaatcacataatcacgaaagccaaaccacatcactgtgatcatgatgagaagggccccgataatcatcaggccatcatcccctccccctttccgttgtggctggttttgcatcagaacctcagagaattttggtaaaccttcccgatataccaggccttacgcgccggcgtattggagtgatagcggccgacaccctgccagaaatcccccccttcgagaatgatgttgtatttgaggatgtaggcactggccattgcagaggcgcaaaagtcgtgctgcagctgctccgctctcacaccaaaatcagccgccagcttattcgcccagatggtgtttatctggaatgcgccgtaatcgtaagtaccgtttttgtttttatttttcattcccggtttcccgccttcagtcagccacaaggcgaggaatccacggagaggaatgtgataaatctgtgctgcttcaaccatgcaggttggcagcgggattgagggcggggcatcaaccataacaatctcctaatgatttctatttcgagtaaaaaatataccatcaaaaggagatttttttaaatcattttggtgcgcataattgagagggtgtcagaggaattttatttgccttcgacagagacaatgttgccctcattcgcattttcccatgtcgggatttcgccactcagcgtgcagcgaagcgtctcttttccgacggtaagccgtgcgccactgctgtagtaatgactttcatacaggcaggcggttttcttttgttccggggtcagtgtaagactggcattctgatcctttgccagcagttgtacaagatgttggtttgtctgtacctgttcacccagaatgctgctgagcaacgaatcggttttcggcgcggatacactctgagcaaaggcactgcaactgattacggatgcggtcagaatgagagcaatcttcgtgttggatttcatttcactttctccattaaaaaaatcggtaatcattttctggatcatttaaattaggtctgaaaaccggcctgagtgtcaagccgattctgtttaaaaattaac